SEQUENCE LISTING (264 sequences)

SEQ ID NO: 1 - protein, 124 aa, synthetic construct
  REGION 1..124: note = A406VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGPTAEASYYYGMDVWGQGTTVTVSS

SEQ ID NO: 2 - protein, 5 aa, synthetic construct
  REGION 1..5: note = A406HCDR1 A4011HCDR1
SYYMH

SEQ ID NO: 3 - protein, 17 aa, synthetic construct
  REGION 1..17: note = A406HCDR2 A4011HCDR2
IINPSGGSTSYAQKFQG

SEQ ID NO: 4 - protein, 15 aa, synthetic construct
  REGION 1..15: note = A406HCDR3
GGPTAEASYYYGMDV

SEQ ID NO: 5 - protein, 111 aa, synthetic construct
  REGION 1..111: note = A406VL
QSALTQPASVSGSPGQSITISCTGTSSDVGGYNHVSWYQQHPGKVPKLMIYEVSNRPSGVSNRFSGSKSGNTASLTISGLQAEDEANYYCSSHTSSGTLWVFGGGTKVTVL

SEQ ID NO: 6 - protein, 14 aa, synthetic construct
  REGION 1..14: note = A406LCDR1
TGTSSDVGGYNHVS

SEQ ID NO: 7 - protein, 7 aa, synthetic construct
  REGION 1..7: note = A406LCDR2
EVSNRPS

SEQ ID NO: 8 - protein, 11 aa, synthetic construct
  REGION 1..11: note = A406LCDR3
SSHTSSGTLWV

SEQ ID NO: 9 - protein, 250 aa, synthetic construct
  REGION 1..250: note = A406scFv
QSALTQPASVSGSPGQSITISCTGTSSDVGGYNHVSWYQQHPGKVPKLMIYEVSNRPSGVSNRFSGSKSGNTASLTISGLQAEDEANYYCSSHTSSGTLWVFGGGTKVTVLGGGGSGGGGSGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGPTAEASYYYGMDVWGQGTTVTVSS

SEQ ID NO: 10 - protein, 119 aa, synthetic construct
  REGION 1..119: note = A409VH
EVQLVESGGGVVLPGRSLRLSCAASGFTLRSYGMHWVRQATGKGLEWVSAIGTAGDTYYPGSVKGRFTISRENAKNSLYLQMNSLRAGDTAVYYCARADRLGGYGMDVWGQGTTVTVSS

SEQ ID NO: 11 - protein, 5 aa, synthetic construct
  REGION 1..5: note = A409HCDR1
SYGMH

SEQ ID NO: 12 - protein, 16 aa, synthetic construct
  REGION 1..16: note = A409HCDR2
AIGTAGDTYYPGSVKG

SEQ ID NO: 13 - protein, 11 aa, synthetic construct
  REGION 1..11: note = A409HCDR3
ADRLGGYGMDV

SEQ ID NO: 14 - protein, 110 aa, synthetic construct
  REGION 1..110: note = A409VL
QAVLTQPPSASGAPGQRVSISCSGGSSNIGSHTVNWYQQLPGAAPRLLMFSNYQRPSGVPDRFSASKSGTSASLAISGLRSEDEADYYCAAWDGTLKGYVFGTGTKVTVL

SEQ ID NO: 15 - protein, 13 aa, synthetic construct
  REGION 1..13: note = A409LCDR1
SGGSSNIGSHTVN

SEQ ID NO: 16 - protein, 7 aa, synthetic construct
  REGION 1..7: note = A409LCDR2
SNYQRPS

SEQ ID NO: 17 - protein, 11 aa, synthetic construct
  REGION 1..11: note = A409LCDR3
AAWDGTLKGYV

SEQ ID NO: 18 - protein, 244 aa, synthetic construct
  REGION 1..244: note = A409scFv
QAVLTQPPSASGAPGQRVSISCSGGSSNIGSHTVNWYQQLPGAAPRLLMFSNYQRPSGVPDRFSASKSGTSASLAISGLRSEDEADYYCAAWDGTLKGYVFGTGTKVTVLGGGGSGGGGSGGGGSEVQLVESGGGVVLPGRSLRLSCAASGFTLRSYGMHWVRQATGKGLEWVSAIGTAGDTYYPGSVKGRFTISRENAKNSLYLQMNSLRAGDTAVYYCARADRLGGYGMDVWGQGTTVTVSS

SEQ ID NO: 19 - protein, 125 aa, synthetic construct
  REGION 1..125: note = A4011VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRSTIFGVVRRAGMDVWGQGTTVTVSS

SEQ ID NO: 20 - protein, 16 aa, synthetic construct
  REGION 1..16: note = A4011HCDR3
DRSTIFGVVRRAGMDV

SEQ ID NO: 21 - protein, 109 aa, synthetic construct
  REGION 1..109: note = A4011VL
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVEIKG

SEQ ID NO: 22 - protein, 12 aa, synthetic construct
  REGION 1..12: note = A4011LCDR1
RASQSVSSSYLA

SEQ ID NO: 23 - protein, 7 aa, synthetic construct
  REGION 1..7: note = A4011LCDR2
GASSRAT

SEQ ID NO: 24 - protein, 9 aa, synthetic construct
  REGION 1..9: note = A4011LCDR3
QQYGSSPYT

SEQ ID NO: 25 - protein, 248 aa, synthetic construct
  REGION 1..248: note = A4011scFv
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVEIKGGGGSGGGGSGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRSTIFGVVRRAGMDVWGQGTTVTVSS

SEQ ID NO: 26 - protein, 117 aa, synthetic construct
  REGION 1..117: note = A4012VH
QVQLVQSGGGVVQPGRSLRLSCAASGFPFRGYAMYWVRQAPGKGLEWVASLSYDGSHEYYGDSVKGRFTISRDNSKNTLYLEMNSLRTEDTAVYYCARQRAYSFDYWGQGTLVTVSS

SEQ ID NO: 27 - protein, 5 aa, synthetic construct
  REGION 1..5: note = A4012HCDR1
GYAMY

SEQ ID NO: 28 - protein, 17 aa, synthetic construct
  REGION 1..17: note = A4012HCDR2
SLSYDGSHEYYGDSVKG

SEQ ID NO: 29 - protein, 8 aa, synthetic construct
  REGION 1..8: note = A4012HCDR3
QRAYSFDY

SEQ ID NO: 30 - protein, 105 aa, synthetic construct
  REGION 1..105: note = A4012VL
QAVLTQPPSVSVSPGQTSSITCSGDNLGHKYASWYQQKPGQSPILVIYQDNKRPSGIPERFSGSNSGNTATLTISGTQAVDEADYYCQVWSSGTVFGGGTKLTVL

SEQ ID NO: 31 - protein, 11 aa, synthetic construct
  REGION 1..11: note = A4012LCDR1
SGDNLGHKYAS

SEQ ID NO: 32 - protein, 7 aa, synthetic construct
  REGION 1..7: note = A4012LCDR2
QDNKRPS

SEQ ID NO: 33 - protein, 8 aa, synthetic construct
  REGION 1..8: note = A4012LCDR3
QVWSSGTV

SEQ ID NO: 34 - protein, 237 aa, synthetic construct
  REGION 1..237: note = A4012scFv
QAVLTQPPSVSVSPGQTSSITCSGDNLGHKYASWYQQKPGQSPILVIYQDNKRPSGIPERFSGSNSGNTATLTISGTQAVDEADYYCQVWSSGTVFGGGTKLTVLGGGGSGGGGSGGGGSQVQLVQSGGGVVQPGRSLRLSCAASGFPFRGYAMYWVRQAPGKGLEWVASLSYDGSHEYYGDSVKGRFTISRDNSKNTLYLEMNSLRTEDTAVYYCARQRAYSFDYWGQGTLVTVSS

SEQ ID NO: 35 - protein, 116 aa, synthetic construct
  REGION 1..116: note = A4021VH
QVQLQESGGGVVQPGGSLRLSCATSGFTFSSYAMSWVRQAPGKGLEWVSAISGSGGNTYYADSVKGRFTISRDNSKNTLYLQMNSLRAEDTAVYYCAKGRNWFDPWGQGTLVTVSS

SEQ ID NO: 36 - protein, 5 aa, synthetic construct
  REGION 1..5: note = A4021HCDR1
SYAMS

SEQ ID NO: 37 - protein, 17 aa, synthetic construct
  REGION 1..17: note = A4021HCDR2
AISGSGGNTYYADSVKG

SEQ ID NO: 38 - protein, 7 aa, synthetic construct
  REGION 1..7: note = A4021HCDR3
GRNWFDP

SEQ ID NO: 39 - protein, 107 aa, synthetic construct
  REGION 1..107: note = A4025VH A4044VH
DIRMTQSPSSVSASVGDRVTITCRASQGIGSWLAWYQQKAGKAPKLLIYAASSLQSGVPSRFSGSGSGTDFTLTISSLQPEDFATYYCQQFNSFPWTFGQGTKVEIK

SEQ ID NO: 40 - protein, 11 aa, synthetic construct
  REGION 1..11: note = A4025HCDR1 A4044HCDR1
RASQGIGSWLA

SEQ ID NO: 41 - protein, 7 aa, synthetic construct
  REGION 1..7: note = A4025HCDR2 A4044HCDR2
AASSLQS

SEQ ID NO: 42 - protein, 9 aa, synthetic construct
  REGION 1..9: note = A4025HCDR3 A4044HCDR3
QQFNSFPWT

SEQ ID NO: 43 - protein, 238 aa, synthetic construct
  REGION 1..238: note = A4025VL
DIRMTQSPSSVSASVGDRVTITCRASQGIGSWLAWYQQKAGKAPKLLIYAASSLQSGVPSRFSGSGSGTDFTLTISSLQPEDFATYYCQQFNSFPWTFGQGTKVEIKGGGGSGGGGSGGGGSQVQLQESGGGVVQPGGSLRLSCATSGFTFSSYAMSWVRQAPGKGLEWVSAISGSGGNTYYADSVKGRFTISRDNSKNTLYLQMNSLRAEDTAVYYCAKGRNWFDPWGQGTLVTVSS

SEQ ID NO: 44 - protein, 116 aa, synthetic construct
  REGION 1..116: note = A4025VH A4044VH
QVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSS

SEQ ID NO: 45 - protein, 5 aa, synthetic construct
  REGION 1..5: note = A4025HCDR1 A4044HCDR1
SNWMS

SEQ ID NO: 46 - protein, 17 aa, synthetic construct
  REGION 1..17: note = A4025HCDR2 A4044HCDR2
NINQDGSEKYYVDSVKG

SEQ ID NO: 47 - protein, 7 aa, synthetic construct
  REGION 1..7: note = A4025HCDR3 A4044HCDR3
GGYRYAY

SEQ ID NO: 48 - protein, 110 aa, synthetic construct
  REGION 1..110: note = A4025VL
QSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVL

SEQ ID NO: 49 - protein, 14 aa, synthetic construct
  REGION 1..14: note = A4025LCDR1
TGTRSDIGDYNYVS

SEQ ID NO: 50 - protein, 7 aa, synthetic construct
  REGION 1..7: note = A4025LCDR2
DVSSRPS

SEQ ID NO: 51 - protein, 10 aa, synthetic construct
  REGION 1..10: note = A4025LCDR3 A4044LCDR3
SSYTSSSTLV

SEQ ID NO: 52 - protein, 241 aa, synthetic construct
  REGION 1..241: note = A4025scFv
QSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSS

SEQ ID NO: 53 - protein, 120 aa, synthetic construct
  REGION 1..120: note = A4034VH
EVHLVESGPGLVKASQTLSLTCTVSGDSISSGGYYWTWIRHHPGRGLEWIGHISYSGRTSYNPSLKSPITISVDTSKNQFSLRLSSVTAADTALYFCARASTRFGPFDYWGQGSLVTVSS

SEQ ID NO: 54 - protein, 7 aa, synthetic construct
  REGION 1..7: note = A4034HCDR1
SGGYYWT

SEQ ID NO: 55 - protein, 16 aa, synthetic construct
  REGION 1..16: note = A4034HCDR2
HISYSGRTSYNPSLKS

SEQ ID NO: 56 - protein, 10 aa, synthetic construct
  REGION 1..10: note = A4034HCDR3
ASTRFGPFDY

SEQ ID NO: 57 - protein, 107 aa, synthetic construct
  REGION 1..107: note = A4034VL
DIRLTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIK

SEQ ID NO: 58 - protein, 11 aa, synthetic construct
  REGION 1..11: note = A4034LCDR1
RASQGISSWLA

SEQ ID NO: 59 - protein, 9 aa, synthetic construct
  REGION 1..9: note = A4034LCDR3
QQANSFPLT

SEQ ID NO: 60 - protein, 242 aa, synthetic construct
  REGION 1..242: note = A4034scFv
DIRLTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIKGGGGSGGGGSGGGGSEVHLVESGPGLVKASQTLSLTCTVSGDSISSGGYYWTWIRHHPGRGLEWIGHISYSGRTSYNPSLKSPITISVDTSKNQFSLRLSSVTAADTALYFCARASTRFGPFDYWGQGSLVTVSS

SEQ ID NO: 61 - protein, 120 aa, synthetic construct
  REGION 1..120: note = A4037VH
EVQLVESGPGLVKPSQTLSLTCPVSGGSISSGTYYWSWIRQPAGKGLEWIGRISSSGSTNYSPSLKSRVTISVDTSRNQFSLKLNSVTAADTALYYCAREDRWGLGFDIWGQGTTVTVSS

SEQ ID NO: 62 - protein, 7 aa, synthetic construct
  REGION 1..7: note = A4037 HCDR1
SGTYYWS

SEQ ID NO: 63 - protein, 16 aa, synthetic construct
  REGION 1..16: note = A4037 HCDR2
RISSSGSTNYSPSLKS

SEQ ID NO: 64 - protein, 10 aa, synthetic construct
  REGION 1..10: note = A4037 HCDR3
EDRWGLGFDI

SEQ ID NO: 65 - protein, 110 aa, synthetic construct
  REGION 1..110: note = A4037 VL
QSALTQPAAVSGSPGQSITISCSGTSSDVGGYNYVSWYQQHPGKAPKLMIFDVSKRPSGVSNRFSGSKSGNTASLTISGLQTEDEADYYCSSYTSSRTYVFGTGTKLTVL

SEQ ID NO: 66 - protein, 14 aa, synthetic construct
  REGION 1..14: note = A4037 LCDR1
SGTSSDVGGYNYVS

SEQ ID NO: 67 - protein, 7 aa, synthetic construct
  REGION 1..7: note = A4037 LCDR2
DVSKRPS

SEQ ID NO: 68 - protein, 10 aa, synthetic construct
  REGION 1..10: note = A4037 LCDR3
SSYTSSRTYV

SEQ ID NO: 69 - protein, 245 aa, synthetic construct
  REGION 1..245: note = A4037 scFv
QSALTQPAAVSGSPGQSITISCSGTSSDVGGYNYVSWYQQHPGKAPKLMIFDVSKRPSGVSNRFSGSKSGNTASLTISGLQTEDEADYYCSSYTSSRTYVFGTGTKLTVLGGGGSGGGGSGGGGSEVQLVESGPGLVKPSQTLSLTCPVSGGSISSGTYYWSWIRQPAGKGLEWIGRISSSGSTNYSPSLKSRVTISVDTSRNQFSLKLNSVTAADTALYYCAREDRWGLGFDIWGQGTTVTVSS

SEQ ID NO: 70 - protein, 110 aa, synthetic construct
  REGION 1..110: note = A4044 VL
QSALTQPASVSGSPGQSITISCTGTSSDVGGYNYVSWYQQHPGKAPKLIIYDVTNRPSGVSNRFSGSKSANTASLTISGLQAEDEADYYCSSYTSSSTLVFGGGTELTVL

SEQ ID NO: 71 - protein, 14 aa, synthetic construct
  REGION 1..14: note = A4044 LCDR1
TGTSSDVGGYNYVS

SEQ ID NO: 72 - protein, 7 aa, synthetic construct
  REGION 1..7: note = A4044 LCDR2
DVTNRPS

SEQ ID NO: 73 - protein, 241 aa, synthetic construct
  REGION 1..241: note = A4044 scFv
QSALTQPASVSGSPGQSITISCTGTSSDVGGYNYVSWYQQHPGKAPKLIIYDVTNRPSGVSNRFSGSKSANTASLTISGLQAEDEADYYCSSYTSSSTLVFGGGTELTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSS

SEQ ID NO: 74 - protein, 121 aa, synthetic construct
  REGION 1..121: note = A4045 VH
EVHLVESGGGVVQPGRSLRLSCAASGFPFSRYGMHWVRQAPGKGLEWVSYISSSGSTIYYADSVKGRFTISRDNSKNTVSLQMNSLRADDTAVYYCSRGTRPGITGRLGYWGQGTLVTVSS

SEQ ID NO: 75 - protein, 5 aa, synthetic construct
  REGION 1..5: note = A4045 HCDR1
RYGMH

SEQ ID NO: 76 - protein, 17 aa, synthetic construct
  REGION 1..17: note = A4045 HCDR2
YISSSGSTIYYADSVKG

SEQ ID NO: 77 - protein, 12 aa, synthetic construct
  REGION 1..12: note = A4045 HCDR3
GTRPGITGRLGY

SEQ ID NO: 78 - protein, 112 aa, synthetic construct
  REGION 1..112: note = A4045 VL
QSALTQPASVSGSPGQSITISCTGTSSDVGGYKYVSWYQQHPGKAPKLMIYDVSNRPSGVSNRFSGSKSGNTASLTISGLQAEDEADYYCSSYTSSSTPHVVFGGGTKVTVL

SEQ ID NO: 79 - protein, 14 aa, synthetic construct
  REGION 1..14: note = A4045 LCDR1
TGTSSDVGGYKYVS

SEQ ID NO: 80 - protein, 7 aa, synthetic construct
  REGION 1..7: note = A4045 LCDR2
DVSNRPS

SEQ ID NO: 81 - protein, 12 aa, synthetic construct
  REGION 1..12: note = A4045 LCDR3
SSYTSSSTPHVV

SEQ ID NO: 82 - protein, 248 aa, synthetic construct
  REGION 1..248: note = A4045 scFv
QSALTQPASVSGSPGQSITISCTGTSSDVGGYKYVSWYQQHPGKAPKLMIYDVSNRPSGVSNRFSGSKSGNTASLTISGLQAEDEADYYCSSYTSSSTPHVVFGGGTKVTVLGGGGSGGGGSGGGGSEVHLVESGGGVVQPGRSLRLSCAASGFPFSRYGMHWVRQAPGKGLEWVSYISSSGSTIYYADSVKGRFTISRDNSKNTVSLQMNSLRADDTAVYYCSRGTRPGITGRLGYWGQGTLVTVSS

SEQ ID NO: 83 - protein, 115 aa, synthetic construct
  REGION 1..115: note = VH A4052
QVQLVQSGAEVKKPGSSVKVSCKASGGTFSSYAISWVRQAPGQGLEWMGMINPSGGTTSYAQKFQGRVTMTRDTSTSTVYMELSSLRPEDTAVYYCARGFRMDQWGQGTLVTVSS

SEQ ID NO: 84 - protein, 5 aa, synthetic construct
  REGION 1..5: note = A4052 HCDR1
SYAIS

SEQ ID NO: 85 - protein, 17 aa, synthetic construct
  REGION 1..17: note = A4052 HCDR2
MINPSGGTTSYAQKFQG

SEQ ID NO: 86 - protein, 6 aa, synthetic construct
  REGION 1..6: note = A4052 HCDR3
GFRMDQ

SEQ ID NO: 87 - protein, 110 aa, synthetic construct
  REGION 1..110: note = A4052 VL
QSALTQPPSASGSPGQSVTISCTGTSSDVGAYNFVSWYQQHPGNAPKLIIYDVRKRPSGIPDRFSASKSGNTASLTVSGLQADDEADYYCSSYAGNNNLVFGGGTKLTVL

SEQ ID NO: 88 - protein, 14 aa, synthetic construct
  REGION 1..14: note = A4052 LCDR1
TGTSSDVGAYNFVS

SEQ ID NO: 89 - protein, 7 aa, synthetic construct
  REGION 1..7: note = A4052 LCDR2
DVRKRPS

SEQ ID NO: 90 - protein, 10 aa, synthetic construct
  REGION 1..10: note = A4052 LCDR3
SSYAGNNNLV

SEQ ID NO: 91 - protein, 240 aa, synthetic construct
  REGION 1..240: note = A4052 scFv
QSALTQPPSASGSPGQSVTISCTGTSSDVGAYNFVSWYQQHPGNAPKLIIYDVRKRPSGIPDRFSASKSGNTASLTVSGLQADDEADYYCSSYAGNNNLVFGGGTKLTVLGGGGSGGGGSGGGGSQVQLVQSGAEVKKPGSSVKVSCKASGGTFSSYAISWVRQAPGQGLEWMGMINPSGGTTSYAQKFQGRVTMTRDTSTSTVYMELSSLRPEDTAVYYCARGFRMDQWGQGTLVTVSS

SEQ ID NO: 92 - protein, 107 aa, synthetic construct
  REGION 1..107: note = CD28-TM/ICD
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 93 - DNA, 321 bp, synthetic construct
  misc_feature 1..321: note = CD28-TM/IC Nucleotide sequence
  source 1..321: mol_type = other DNA
attgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 94 - protein, 120 aa, synthetic construct
  REGION 1..120: note = 1412 heavy chain variable domain (CD28 antibody VH)
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 95 - protein, 110 aa, synthetic construct
  REGION 1..110: note = 1412 light chain variable domain (CD28 antibody VL)
VMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 96 - protein, 266 aa, synthetic construct
  REGION 1..266: note = 1412 scFv (CD28 antibodyscFv)
ALPVTALLLPLALLLHAARPQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSVEGGSGGSGGSGGSGGVMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 97 - protein, 5 aa, synthetic construct
  REGION 1..5: note = linker
GGGGS

SEQ ID NO: 98 - protein, 5 aa, synthetic construct
  REGION 1..5: note = linker
EAAAK

SEQ ID NO: 99 - skipped

SEQ ID NO: 100 - protein, 15 aa, synthetic construct
  REGION 1..15: note = linker
GGGGSGGGGSGGGGS

SEQ ID NO: 101 - protein, 22 aa, synthetic construct
  REGION 1..22: note = F2A
VKQTLNFDLLKLAGDVESNPGP

SEQ ID NO: 102 - protein, 5 aa, synthetic construct
  REGION 1..5: note = 70.202 HCDR1
NYYWS

SEQ ID NO: 103 - protein, 16 aa, synthetic construct
  REGION 1..16: note = 70.202 HCDR2
YIYYSGGSNYNPSLRS

SEQ ID NO: 104 - protein, 12 aa, synthetic construct
  REGION 1..12: note = 70.202 HCDR3
GPDLGTTADFDY

SEQ ID NO: 105 - protein, 13 aa, synthetic construct
  REGION 1..13: note = 70.202 LCDR1
SGSRSNIGNNAVN

SEQ ID NO: 106 - protein, 7 aa, synthetic construct
  REGION 1..7: note = 70.202 LCDR2
YDDLLPS

SEQ ID NO: 107 - protein, 11 aa, synthetic construct
  REGION 1..11: note = 70.202 LCDR3
AAWDDSLNGVV

SEQ ID NO: 108 - protein, 120 aa, synthetic construct
  REGION 1..120: note = 70.202 VH
QLQLQESGPGLVKPSETLSLTCVVSGDSMNNYYWSWIRQSPGKGLEWIGYIYYSGGSNYNPSLRSRVTMSVDTAKNQFSLNLRSVTAADTAIYYCVRGPDLGTTADFDYWGQGTLVTVSS

SEQ ID NO: 109 - protein, 110 aa, synthetic construct
  REGION 1..110: note = 70.202 VL
QPVLTQPPLVSEAPRQRVTISCSGSRSNIGNNAVNWYQQLPGKAPKLLIYYDDLLPSGVSDRFSGSKSGTSASLAISGLQPEDEADYYCAAWDDSLNGVVFGGGTELTVL

SEQ ID NO: 110 - protein, 245 aa, synthetic construct
  REGION 1..245: note = 70.202 scFv
QPVLTQPPLVSEAPRQRVTISCSGSRSNIGNNAVNWYQQLPGKAPKLLIYYDDLLPSGVSDRFSGSKSGTSASLAISGLQPEDEADYYCAAWDDSLNGVVFGGGTELTVLGGGGSGGGGSGGGGSQLQLQESGPGLVKPSETLSLTCVVSGDSMNNYYWSWIRQSPGKGLEWIGYIYYSGGSNYNPSLRSRVTMSVDTAKNQFSLNLRSVTAADTAIYYCVRGPDLGTTADFDYWGQGTLVTVSS

SEQ ID NO: 111 - protein, 489 aa, synthetic construct
  REGION 1..489: note = 70.202 CAR
MALPVTALLLPLALLLHAARPQPVLTQPPLVSEAPRQRVTISCSGSRSNIGNNAVNWYQQLPGKAPKLLIYYDDLLPSGVSDRFSGSKSGTSASLAISGLQPEDEADYYCAAWDDSLNGVVFGGGTELTVLGGGGSGGGGSGGGGSQLQLQESGPGLVKPSETLSLTCVVSGDSMNNYYWSWIRQSPGKGLEWIGYIYYSGGSNYNPSLRSRVTMSVDTAKNQFSLNLRSVTAADTAIYYCVRGPDLGTTADFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - DNA, 1470 bp, synthetic construct
  misc_feature 1..1470: note = 70.202 CAR mRNA
  source 1..1470: mol_type = other DNA
atggccttaccagtgaccgccttgctcctgccgctggccttgctgctccacgccgccaggccgcagccagtgctgactcagccacccttggtgtctgaagcccccaggcagagggtcaccatctcctgttctggaagcaggtccaacatcggaaataatgctgtaaattggtaccagcagctcccaggaaaggctcccaaactcctcatctattatgacgatctgctgccctcaggggtctctgaccgattctctggctccaagtctggcacctcagcctccctggccatcagtgggctccagcctgaggatgaggctgattattactgtgcagcatgggatgacagcctgaatggtgtggtattcggcggagggaccgagctgaccgtcctaggtggtggtggttctggcggcggcggctccggaggtggtggatcccagctgcagctgcaggagtcgggcccaggactggtgaagccttcggagaccctgtccctcacctgcgttgtctctggtgactccatgaataattactactggagctggatccggcagtccccaggaaagggactggagtggattggttacatctattacagtgggggctccaactacaacccctccctcaggagtcgagtcaccatgtcagtagacacggccaagaaccagttctccctgaatctgaggtctgtcactgctgcggacacggccatttattactgtgtgagagggcccgacctggggacaacggctgattttgactactggggccagggaaccctggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 113 - protein, 5 aa, synthetic construct
  REGION 1..5: note = MESO HCDR1
ANTIN

SEQ ID NO: 114 - protein, 17 aa, synthetic construct
  REGION 1..17: note = MESO HCDR2
RIIPSLNIRDYAQEFQG

SEQ ID NO: 115 - protein, 14 aa, synthetic construct
  REGION 1..14: note = MESO HCDR3
DPGSTWSPNQFFPH

SEQ ID NO: 116 - protein, 11 aa, synthetic construct
  REGION 1..11: note = MESO LCDR1
RASQGISNSLA

SEQ ID NO: 117 - protein, 7 aa, synthetic construct
  REGION 1..7: note = MESO LCDR2
AASRLES

SEQ ID NO: 118 - protein, 9 aa, synthetic construct
  REGION 1..9: note = MESO LCDR3
QQYYSTPFT

SEQ ID NO: 119 - protein, 123 aa, synthetic construct
  REGION 1..123: note = MESO VH
QVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSS

SEQ ID NO: 120 - protein, 107 aa, synthetic construct
  REGION 1..107: note = MESO VL
AIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIK

SEQ ID NO: 121 - protein, 245 aa, synthetic construct
  REGION 1..245: note = MESO scFv
AIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSS

SEQ ID NO: 122 - protein, 489 aa, synthetic construct
  REGION 1..489: note = MESO CAR
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1470 bp, synthetic construct
  misc_feature 1..1470: note = MESO CAR DNA
  source 1..1470: mol_type = other DNA
atggccttaccagtgaccgccttgctcctgccgctggccttgctgctccacgccgccaggccggccatccggatgacccagtctccatcctccctgtctgcatctgtaggagacagagtcaccatcacttgccgggcgagtcagggcattagcaattctttagcctggtatcagcagaaaccagggaaagcccctaagctcctgctctatgctgcatccagattggaaagtggggtcccatccaggttcagtggcagtggatctgggacggattacactctcaccatcagcagcctgcagcctgaagattttgcaacttattactgtcaacagtattatagtacccccttcactttcggccctgggaccaagctggagatcaaaggtggtggtggttctggcggcggcggctccggaggtggtggatcccaggtgcagctggtacagtctggggctgaagtgaagaagcctgggtcctcggtgaaggtctcctgcaagacttctgcagacaccttcagcgcaaatactatcaactgggtgcgacaggcccctggacacgggcttgagtggatgggaaggatcatcccttctctgaatatacgagactacgcacaggagttccagggcagagtcacatttaccgcggacgaatccacgaacacagtctatatgcagttgaccagcctgagatctgaggacacggccgtgtattactgtgcgagagatccgggcagcacctggtccccgaatcaattcttcccccactggggccagggcaccctggtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 124 - protein, 21 aa, synthetic construct
  REGION 1..21: note = CD8 signal peptide
MALPVTALLLPLALLLHAARP

SEQ ID NO: 125 - DNA, 63 bp, synthetic construct
  misc_feature 1..63: note = CD8 signal peptide DNA
  source 1..63: mol_type = other DNA
atggccttaccagtgaccgccttgctcctgccgctggccttgctgctccacgccgccaggccg

SEQ ID NO: 126 - protein, 45 aa, synthetic construct
  REGION 1..45: note = CD8 Hinge
TTTPAPRPPTPAPTIASQPLSLRPEACRPAAGGAVHTRGLDFACD

SEQ ID NO: 127 - DNA, 135 bp, synthetic construct
  misc_feature 1..135: note = CD8 Hinge DNA
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 128 - protein, 24 aa, synthetic construct
  REGION 1..24: note = CD8 transmembrane domain
IYIWAPLAGTCGVLLLSLVITLYC

SEQ ID NO: 129 - DNA, 72 bp, synthetic construct
  misc_feature 1..72: note = CD8 transmembrane domain DNA
  source 1..72: mol_type = other DNA
atctacatctgggcgcccttggccgggacttgtggggtccttctcctgtcactggttatcaccctttactgc

SEQ ID NO: 130 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 131 - DNA, 126 bp, synthetic construct
  misc_feature 1..126: note = 4-1BB costimulatory domain DNA
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 132 - protein, 112 aa, synthetic construct
  REGION 1..112: note = CD3-zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - DNA, 336 bp, synthetic construct
  misc_feature 1..336: note = CD3-zeta DNA
  source 1..336: mol_type = other DNA
agagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 134 - DNA, 22 bp, synthetic construct
  misc_feature 1..22: note = Forward primer sequence
  source 1..22: mol_type = other DNA
tgcagctggcacgacaggtttc

SEQ ID NO: 135 - DNA, 19 bp, synthetic construct
  misc_feature 1..19: note = reverse primer sequence
  source 1..19: mol_type = other DNA
cgtcagactgtagcacgtt

SEQ ID NO: 136 - DNA, 20 bp, synthetic construct
  misc_feature 1..20: note = Forward primer sequence
  source 1..20: mol_type = other DNA
aacaattgaattcaggagga

SEQ ID NO: 137 - DNA, 19 bp, synthetic construct
  misc_feature 1..19: note = reverse primer sequence
  source 1..19: mol_type = other DNA
cctcctaagaagcgtagtc

SEQ ID NO: 138 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 139 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 140 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 141 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 142 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 143 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 144 - protein, 17 aa, synthetic construct
  REGION 1..17: note = GSGGGGSGGGGSGGGGS linker
GSGGGGSGGGGSGGGGS

SEQ ID NO: 145 - protein, 43 aa, synthetic construct
  REGION 1..43: note = CD28 Hinge
VAAAIEVMYPPPYLDNEKSNGTIIHVKGKHLCPSPLFPGPSKP

SEQ ID NO: 146 - protein, 35 aa, synthetic construct
  REGION 1..35: note = IgG1 Hinge
AKTTPPSVYGRVTVSSAEPKSCDKTHTCPPCPDPK

SEQ ID NO: 147 - protein, 27 aa, synthetic construct
  REGION 1..27: note = T4 fibritin trimerization motif
GYIPEAPRDGQAYVRKDGEWVLLSTFL

SEQ ID NO: 148 - protein, 30 aa, synthetic construct
  REGION 1..30: note = Isoleucine Zipper (L)
EKKIEAIEKKIEAIEKKIEAIEAIEKKIEA

SEQ ID NO: 149 - protein, 20 aa, synthetic construct
  REGION 1..20: note = Isoleucine Zipper (S)
EKKIEAIEKKIEAIEKKIEA

SEQ ID NO: 150 - protein, 28 aa, synthetic construct
  REGION 1..28: note = GCN4II
EDKIEEILSKIYHIENEIARIKKLIGEA

SEQ ID NO: 151 - protein, 28 aa, synthetic construct
  REGION 1..28: note = Optimized GCN4II
EDKVEELLSKIYHIENRIARIEKLVGEA

SEQ ID NO: 152 - protein, 43 aa, synthetic construct
  REGION 1..43: note = Matrilin -1 (L)
EEDPCECKSIVKFQTKVEELINTLQQKLEAVAKRIEALENKII

SEQ ID NO: 153 - protein, 26 aa, synthetic construct
  REGION 1..26: note = Matrilin -1 (S)
EELINTLQQKLEAVAKRIEALENKII

SEQ ID NO: 154 - protein, 64 aa, synthetic construct
  REGION 1..64: note = Human collagen XV trimerization domain
VTAFSNMDDMLQKAHLVIEGTFIYLRDSTEFFIRVRDGWKKLQLGELIPIPADSPPPPALSSNP

SEQ ID NO: 155 - protein, 357 aa, synthetic construct
  REGION 1..357: note = A406C2828
QSALTQPASVSGSPGQSITISCTGTSSDVGGYNHVSWYQQHPGKVPKLMIYEVSNRPSGVSNRFSGSKSGNTASLTISGLQAEDEANYYCSSHTSSGTLWVFGGGTKVTVLGGGGSGGGGSGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGPTAEASYYYGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 156 - protein, 351 aa, synthetic construct
  REGION 1..351: note = A409C2828
QAVLTQPPSASGAPGQRVSISCSGGSSNIGSHTVNWYQQLPGAAPRLLMFSNYQRPSGVPDRFSASKSGTSASLAISGLRSEDEADYYCAAWDGTLKGYVFGTGTKVTVLGGGGSGGGGSGGGGSEVQLVESGGGVVLPGRSLRLSCAASGFTLRSYGMHWVRQATGKGLEWVSAIGTAGDTYYPGSVKGRFTISRENAKNSLYLQMNSLRAGDTAVYYCARADRLGGYGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 157 - protein, 355 aa, synthetic construct
  REGION 1..355: note = A4011C2828
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVEIKGGGGSGGGGSGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRSTIFGVVRRAGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 158 - protein, 344 aa, synthetic construct
  REGION 1..344: note = A4012C2828
QAVLTQPPSVSVSPGQTSSITCSGDNLGHKYASWYQQKPGQSPILVIYQDNKRPSGIPERFSGSNSGNTATLTISGTQAVDEADYYCQVWSSGTVFGGGTKLTVLGGGGSGGGGSGGGGSQVQLVQSGGGVVQPGRSLRLSCAASGFPFRGYAMYWVRQAPGKGLEWVASLSYDGSHEYYGDSVKGRFTISRDNSKNTLYLEMNSLRTEDTAVYYCARQRAYSFDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 159 - protein, 345 aa, synthetic construct
  REGION 1..345: note = A4021C2828
DIRMTQSPSSVSASVGDRVTITCRASQGIGSWLAWYQQKAGKAPKLLIYAASSLQSGVPSRFSGSGSGTDFTLTISSLQPEDFATYYCQQFNSFPWTFGQGTKVEIKGGGGSGGGGSGGGGSQVQLQESGGGVVQPGGSLRLSCATSGFTFSSYAMSWVRQAPGKGLEWVSAISGSGGNTYYADSVKGRFTISRDNSKNTLYLQMNSLRAEDTAVYYCAKGRNWFDPWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 160 - protein, 348 aa, synthetic construct
  REGION 1..348: note = A4025C2828
QSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 161 - protein, 349 aa, synthetic construct
  REGION 1..349: note = A4034C2828
DIRLTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIKGGGGSGGGGSGGGGSEVHLVESGPGLVKASQTLSLTCTVSGDSISSGGYYWTWIRHHPGRGLEWIGHISYSGRTSYNPSLKSPITISVDTSKNQFSLRLSSVTAADTALYFCARASTRFGPFDYWGQGSLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 162 - protein, 352 aa, synthetic construct
  REGION 1..352: note = A4037C2828
QSALTQPAAVSGSPGQSITISCSGTSSDVGGYNYVSWYQQHPGKAPKLMIFDVSKRPSGVSNRFSGSKSGNTASLTISGLQTEDEADYYCSSYTSSRTYVFGTGTKLTVLGGGGSGGGGSGGGGSEVQLVESGPGLVKPSQTLSLTCPVSGGSISSGTYYWSWIRQPAGKGLEWIGRISSSGSTNYSPSLKSRVTISVDTSRNQFSLKLNSVTAADTALYYCAREDRWGLGFDIWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 163 - protein, 348 aa, synthetic construct
  REGION 1..348: note = A4044C2828
QSALTQPASVSGSPGQSITISCTGTSSDVGGYNYVSWYQQHPGKAPKLIIYDVTNRPSGVSNRFSGSKSANTASLTISGLQAEDEADYYCSSYTSSSTLVFGGGTELTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 164 - protein, 355 aa, synthetic construct
  REGION 1..355: note = A4045C2828
QSALTQPASVSGSPGQSITISCTGTSSDVGGYKYVSWYQQHPGKAPKLMIYDVSNRPSGVSNRFSGSKSGNTASLTISGLQAEDEADYYCSSYTSSSTPHVVFGGGTKVTVLGGGGSGGGGSGGGGSEVHLVESGGGVVQPGRSLRLSCAASGFPFSRYGMHWVRQAPGKGLEWVSYISSSGSTIYYADSVKGRFTISRDNSKNTVSLQMNSLRADDTAVYYCSRGTRPGITGRLGYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 165 - protein, 347 aa, synthetic construct
  REGION 1..347: note = A4052C2828
QSALTQPPSASGSPGQSVTISCTGTSSDVGAYNFVSWYQQHPGNAPKLIIYDVRKRPSGIPDRFSASKSGNTASLTVSGLQADDEADYYCSSYAGNNNLVFGGGTKLTVLGGGGSGGGGSGGGGSQVQLVQSGAEVKKPGSSVKVSCKASGGTFSSYAISWVRQAPGQGLEWMGMINPSGGTTSYAQKFQGRVTMTRDTSTSTVYMELSSLRPEDTAVYYCARGFRMDQWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 166 - DNA, 1071 bp, synthetic construct
  misc_feature 1..1071: note = A406C2828 DNA Sequence
  source 1..1071: mol_type = other DNA
caatctgccctgactcagcctgcctccgtgtctgggtctcctggacagtcgatcaccatctcctgcactggaaccagcagtgacgttggtggttataatcatgtctcctggtaccaacagcacccaggcaaagtccccaaactcatgatttatgaagtcagtaatcggccctcaggggtttctaatcgcttctctggctccaagtctggcaacacggcctccctgaccatctctgggctccaggctgaggacgaggctaattattactgcagctcacatacaagcagcggcactctttgggtgttcggcggagggaccaaggtcaccgtcctaggtggtggtggttctggcggcggcggctccggaggtggtggatcccaggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggcggtcctacggccgaagctagttactactacggtatggacgtctggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 167 - DNA, 1053 bp, synthetic construct
  misc_feature 1..1053: note = A409C2828 DNA Sequence
  source 1..1053: mol_type = other DNA
caggcagtgctgactcagccaccctcagcgtctggagcccccgggcagagggtctccatctcttgctctggaggcagctccaacatcggaagtcatactgtcaactggtaccagcaactcccaggagcggcccccagactcctcatgttcagtaattatcagcggccctcgggggtccctgaccgattctctgcctccaagtctggcacctcggcctccctggccattagtgggctccggtctgaggatgaggctgattattactgtgcagcatgggacggcaccctgaaaggatatgtcttcggcactggcaccaaggtgaccgtcctcggtggtggtggttctggcggcggcggctccggaggtggtggatccgaggtgcagctggtggagtctgggggaggcgtggtcctgcctgggaggtccctgagactctcctgtgcagcgtctggattcaccctcaggagctatggcatgcactgggtccgccaagctacaggaaaaggtctggagtgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagagcagatagactcgggggctacggtatggacgtctggggccaagggaccacggtcactgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 168 - DNA, 1065 bp, synthetic construct
  misc_feature 1..1065: note = A4011C2828 DNA Sequence
  source 1..1065: mol_type = other DNA
gaaatagtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtacacttttggccaggggaccaaggtggagatcaaaggtggtggtggttctggcggcggcggctccggaggtggtggatcccaggtgcagctggtacagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgctagagatcggagtacgatttttggagtggtccgacgggcgggtatggacgtctggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 169 - DNA, 1032 bp, synthetic construct
  misc_feature 1..1032: note = A4012C2828 DNA Sequence
  source 1..1032: mol_type = other DNA
caggcagtgctgactcagccaccctcagtgtccgtgtccccaggacagacatccagcatcacctgctctggagataatttggggcataagtatgcttcctggtatcagcagaagccgggccagtcccctatcctggtcatctatcaagataacaagcggccctctgggatccctgagcgattctctggctccaactctggaaacacagccactctgaccatcagcgggacccaggctgtggatgaggctgactattactgtcaagtgtggagctccggaacggtcttcggcggagggaccaagctgaccgtcctaggtggtggtggttctggcggcggcggctccggaggtggtggatcccaggtgcagctggtacagtctggggggggcgtggtccagcctgggcggtccctgagactctcctgtgcagcctctggattccccttccgtggctatgctatgtactgggtccgccaggctccgggcaaggggctggagtgggtggcttctctatcatatgatggcagtcacgaatactacggagactccgtgaagggccgattcaccatctccagagacaattccaagaacacgctgtatctggaaatgaacagcctgagaactgaggacacggctgtgtattactgtgcgaggcagagggcttacagttttgattattggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 170 - DNA, 1035 bp, synthetic construct
  misc_feature 1..1035: note = A4021C2828 DNA Sequence
  source 1..1035: mol_type = other DNA
gacatccggatgacccagtctccatcttccgtgtctgcatctgtaggagacagagtcaccatcacttgtcgggcgagtcagggtattggcagctggttagcctggtatcagcagaaagcagggaaagcccctaagctcctgatctatgctgcatccagtttgcaaagtggggtcccatcaaggttcagcggcagtggatctgggacagatttcactctcaccatcagcagcctgcagcctgaagactttgcaacttactattgtcaacagtttaacagtttcccctggacgttcggccaagggaccaaggtggaaatcaaaggtggtggtggttctggcggcggcggctccggaggtggtggatcccaggtgcagctgcaggagtcggggggaggcgtggtccagcctggggggtccctgaggctctcctgtgcaacgtctggattcacctttagcagctatgccatgagctgggtccgccaggctccagggaaggggctggagtgggtctcagctattagtggtagtggtggtaacacatactacgcagactccgtgaagggccggttcaccatctccagagacaattccaagaacacgctgtatctgcaaatgaacagcctgagagccgaggacacggccgtatattactgtgcgaaagggcgaaactggttcgacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 171 - DNA, 1044 bp, synthetic construct
  misc_feature 1..1044: note = A4025C2828 DNA Sequence
  source 1..1044: mol_type = other DNA
cagtctgccctgactcagcccgcctccgtgtctgggtctcctggacagtcgatcaccgtctcctgcactggaaccagaagtgacattggtgattataattatgtctcctggtaccaacaacacccaggcaaagcccccaaactcatcatttatgatgtcagttctcggccctcaggggtttctaatcgctactctggctccaagtctggcaacacggcctccctgaccatctctggcctccaggctgaggacgaggctgattattactgcagctcatatacaagcagcagcactctggtattcggcggagggaccaaggtcaccgtcctaggtggtggtggttctggcggcggcggctccggaggtggtggatcccaggtgcagctggtacagtctgggggaggcttggtccagcctggggggtccctgagactctcctgtgcagcctctggattcacctttagtagcaattggatgagttgggtccgccaggctccagggaaggggctggagtgggtggccaacataaaccaagatggaagtgagaaatactatgtggactctgtgaagggccgattcaccatctccagagacaacgccaagaactcactgtatctgcaactgaacagcctgagagccgaggacacggctgagtattactgtgcgagagggggatacagatatgcctattggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 172 - DNA, 1047 bp, synthetic construct
  misc_feature 1..1047: note = A4034C2828 DNA Sequence
  source 1..1047: mol_type = other DNA
gacatccggttgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctctcactttcggcggagggaccaagctggagatcaaaggtggtggtggttctggcggcggcggctccggaggtggtggatccgaagtgcatctggtggagtcgggcccaggactggtgaaggcttcacagaccctgtccctcacctgcactgtctctggtgactccatcagcagtggtggttactactggacctggatccgccaccacccagggaggggcctggagtggattgggcacatctcttacagtgggcgcacctcctacaacccgtccctcaagagtccaataaccatatcagttgacacttctaagaatcagttctccctgaggctgagctctgtgactgccgcggacacggccttgtatttctgcgcgagagcttcgacccgattcgggccatttgactactggggccagggatccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 173 - DNA, 1056 bp, synthetic construct
  misc_feature 1..1056: note = A4037C2828 DNA Sequence
  source 1..1056: mol_type = other DNA
caatctgccctgactcagcctgccgccgtgtctggatctcctggacagtcgatcaccatctcctgcagtggaaccagcagtgacgttggtggttataactatgtctcctggtaccaacaacacccaggcaaagcccccaaactcatgatttttgatgtcagtaagcggccctcaggggtttctaatcgcttctctggctccaagtctggcaacacggcctccctgaccatctctgggctccagactgaggacgaggctgattattactgcagctcatatacaagcagccgcacttatgtcttcggaactgggaccaagctgaccgtcctaggtggtggtggttctggcggcggcggctccggaggtggtggatccgaagtgcagctggtggagtcgggcccaggactggtgaagccttcacagaccctgtccctcacctgccctgtctctggtggctccatcagcagcggtacttactactggagctggatccggcagcccgccgggaagggactggagtggattgggcgtatctcttccagtgggagcaccaactacagcccctccctcaagagtcgagtcaccatatcagtagacacgtccaggaaccagttctccctgaaactgaactctgtgaccgccgcggacacggccctatattactgtgcgagagaggatcggtggggattaggttttgatatatggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 174 - DNA, 1044 bp, synthetic construct
  misc_feature 1..1044: note = A4044C2828 DNA Sequence
  source 1..1044: mol_type = other DNA
caatctgccctgactcagcctgcctccgtgtctgggtctcctggacagtcgatcaccatctcctgcactggaaccagcagtgacgttggtggttataactatgtctcctggtaccaacaacacccaggcaaagcccccaaactcataatttatgatgtcactaatcggccctccggggtttctaatcgcttctctggctccaagtctgccaacacggcctccctgaccatctctgggctccaggctgaggacgaggctgattattactgcagctcatatacaagcagcagcactttggtgttcggcggagggaccgagctgaccgtcctaggtggtggtggttctggcggcggcggctccggaggtggtggatcccaggtgcagctggtacagtctgggggaggcttggtccagcctggggggtccctgagactctcctgtgcagcctctggattcacctttagtagcaattggatgagttgggtccgccaggctccagggaaggggctggagtgggtggccaacataaaccaagatggaagtgagaaatactatgtggactctgtgaagggccgattcaccatctccagagacaacgccaagaactcactgtatctgcaactgaacagcctgagagccgaggacacggctgagtattactgtgcgagagggggatacagatatgcctattggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 175 - DNA, 1065 bp, synthetic construct
  misc_feature 1..1065: note = A4045C2828 DNA Sequence
  source 1..1065: mol_type = other DNA
caatctgccctgactcagcctgcctccgtgtctgggtctcctggacagtcgatcaccatctcctgcactggaaccagcagtgacgttggtggttataaatatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactccccatgtggtattcggcggagggaccaaggtcaccgtcctaggtggtggtggttctggcggcggcggctccggaggtggtggatccgaggtgcatctggtggagtctgggggaggcgtggtccagcctgggaggtccctgagactctcctgtgcagcgtctggattccccttcagtcgctatggcatgcactgggtccgccaggctccaggcaaggggctggagtgggtttcatacattagtagtagtggtagtaccatatactacgcagactccgtgaagggccgattcaccatctccagagacaattcgaagaacacggtgtctctgcaaatgaatagcctgagagccgacgacacggctgtctattactgttcgagagggacccgtcctggaataactggaagacttggctactggggccagggaaccctggtcactgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 176 - DNA, 1041 bp, synthetic construct
  misc_feature 1..1041: note = A4052C2828 DNA Sequence
  source 1..1041: mol_type = other DNA
cagtctgccctgactcagcctccctccgcgtccgggtctcctggacagtcagtcaccatctcctgcactggaaccagcagtgacgttggtgcttataactttgtctcctggtaccaacagcacccaggcaatgcccccaaactcataatttatgacgtccgtaagcggccctcagggatccctgatcgcttctctgcctccaagtctggcaacacggcctccctgaccgtctctgggctccaggctgacgatgaggctgattattactgcagctcatatgcaggcaacaacaatttggtattcggcggagggaccaagctgaccgtcctaggtggtggtggttctggcggcggcggctccggaggtggtggatcccaggtgcagctggtacagtctggggctgaggtgaagaagcctgggtcctcggtgaaggtctcctgcaaggcttctggaggcaccttcagcagctatgctatcagctgggtgcgacaggcccctggacaagggcttgagtggatgggaatgatcaaccctagtggtggtaccacaagctacgcacagaagttccagggcagagtcaccatgaccagggacacgtccacgagtacagtctacatggagctgagcagcctgagacctgaggacacggccgtgtattactgtgcgagagggtttagaatggaccagtggggccagggaaccctggtcactgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 177 - protein, 5 aa, synthetic construct
  REGION 1..5: note = 9.3h11 HCDR1
DYGVH

SEQ ID NO: 178 - protein, 16 aa, synthetic construct
  REGION 1..16: note = 9.3h11 HCDR2
VIWAGGGTNYNSALMS

SEQ ID NO: 179 - protein, 12 aa, synthetic construct
  REGION 1..12: note = 9.3h11 HCDR3
DKGYSYYYSMDY

SEQ ID NO: 180 - protein, 15 aa, synthetic construct
  REGION 1..15: note = 9.3h11 LCDR1
RASESVEYYVTSLMQ

SEQ ID NO: 181 - protein, 7 aa, synthetic construct
  REGION 1..7: note = 9.3h11 LCDR2
AASNVES

SEQ ID NO: 182 - protein, 9 aa, synthetic construct
  REGION 1..9: note = 9.3h11 LCDR3
QQSRKVPYT

SEQ ID NO: 183 - protein, 120 aa, synthetic construct
  REGION 1..120: note = 9.3h11 VH
QVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSS

SEQ ID NO: 184 - protein, 111 aa, synthetic construct
  REGION 1..111: note = 9.3h11 VL
DIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 185 - protein, 246 aa, synthetic construct
  REGION 1..246: note = 9.3h11 scFv
QVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 186 - protein, 492 aa, synthetic construct
  REGION 1..492: note = sLACO 4025-9.3h11 Amino acid sequence
QSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 187 - protein, 240 aa, synthetic construct
  REGION 1..240: note = Anti-HER2 scFv-H13 Amino acid sequence
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVDIKGGGGSGGGGSGGGGSEVHLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDFGPTNGFDPWGQGTLVTVSS

SEQ ID NO: 188 - protein, 885 aa, synthetic construct
  REGION 1..885: note = H13-A4025-CD28 Amino acid sequence, Membrane
MALPVTALLLPLALLLHAARPDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVDIKGGGGSGGGGSGGGGSEVHLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDFGPTNGFDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGGGGSGGGGSEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 189 - protein, 886 aa, synthetic construct
  REGION 1..886: note = H13-A40517-CD28 Amino acid sequence, Membrane
MALPVTALLLPLALLLHAARPDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVDIKGGGGSGGGGSGGGGSEVHLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDFGPTNGFDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGGGGSGGGGSEGRGSLLTCGDVEENPGPMALPVTALLLPLALLLHAARPQVQLVQSGAEVKKPGSSVKVSCKASGGTFSSYAISWVRQAPGQGLEWMGMINPSGGTTSYAQKFQGRVTMTRDTSTSTVYMELSSLRPEDTAVYYCARGFRMDQWGQGTLVTVSSASGGGGSGGGGSGGGGSQSALTQPPSASGSPGQSVTISCTGTSSDVGAYNFVSWYQQHPGNAPKLIIYDVRKRPSGIPDRFSASKSGNTASLTVSGLQADDEADYYCSSYAGNNEMVFGGGTKLTVLIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 190 - protein, 1029 aa, synthetic construct
  REGION 1..1029: note = H13-A4025-9.3h11 Amino acid sequence, soluble
MALPVTALLLPLALLLHAARPDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVDIKGGGGSGGGGSGGGGSEVHLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDFGPTNGFDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGGGGSGGGGSEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 191 - protein, 1030 aa, synthetic construct
  REGION 1..1030: note = H13-A40517-9.3h11 Amino acid sequence, soluble
MALPVTALLLPLALLLHAARPDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVDIKGGGGSGGGGSGGGGSEVHLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDFGPTNGFDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGGGGSGGGGSEGRGSLLTCGDVEENPGPMALPVTALLLPLALLLHAARPQVQLVQSGAEVKKPGSSVKVSCKASGGTFSSYAISWVRQAPGQGLEWMGMINPSGGTTSYAQKFQGRVTMTRDTSTSTVYMELSSLRPEDTAVYYCARGFRMDQWGQGTLVTVSSASGGGGSGGGGSGGGGSQSALTQPPSASGSPGQSVTISCTGTSSDVGAYNFVSWYQQHPGNAPKLIIYDVRKRPSGIPDRFSASKSGNTASLTVSGLQADDEADYYCSSYAGNNEMVFGGGTKLTVLGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 192 - protein, 244 aa, synthetic construct
  REGION 1..244: note = Anti-MSLN scFv-MH150
QPVLTQPPSASGTPGQRVTISCSGSSSNIGSNYVYWYQQLPGTAPKLLLYNNNQRPSGVPDRFSGSKSVTSASLAISGLRSDDEADYYCATWDDSLSGPVFGGGTKLTVLGGGGSGGGGSGGGGSQVQLVQSGAEVKKPGSSVKVSCKASGGTFSSYAISWVRQAPGQGLEWMGGISAYNGNTNYAQKLQGRVTMTTDTSTSTAYMELRSLRSDDTAVYYCARGGLYSGVFDYWGQGTLVTVSS

SEQ ID NO: 193 - protein, 879 aa, synthetic construct
  REGION 1..879: note = A4025-CD28-MH150 Amino acid sequence, Membrane
MALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSVKQTLNFDLLKLAGDVESNPGPMALPVTALLLPLALLLHAARPQPVLTQPPSASGTPGQRVTISCSGSSSNIGSNYVYWYQQLPGTAPKLLLYNNNQRPSGVPDRFSGSKSVTSASLAISGLRSDDEADYYCATWDDSLSGPVFGGGTKLTVLGGGGSGGGGSGGGGSQVQLVQSGAEVKKPGSSVKVSCKASGGTFSSYAISWVRQAPGQGLEWMGGISAYNGNTNYAQKLQGRVTMTTDTSTSTAYMELRSLRSDDTAVYYCARGGLYSGVFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 194 - protein, 1022 aa, synthetic construct
  REGION 1..1022: note = MH150-A4025-9.3h11 Amino acid sequence, soluble
MALPVTALLLPLALLLHAARPQPVLTQPPSASGTPGQRVTISCSGSSSNIGSNYVYWYQQLPGTAPKLLLYNNNQRPSGVPDRFSGSKSVTSASLAISGLRSDDEADYYCATWDDSLSGPVFGGGTKLTVLGGGGSGGGGSGGGGSQVQLVQSGAEVKKPGSSVKVSCKASGGTFSSYAISWVRQAPGQGLEWMGGISAYNGNTNYAQKLQGRVTMTTDTSTSTAYMELRSLRSDDTAVYYCARGGLYSGVFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 195 - protein, 372 aa, synthetic construct
  REGION 1..372: note = CD27 CAR
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 196 - protein, 864 aa, synthetic construct
  REGION 1..864: note = CD27-CAR-LACO(A4025-9.3h11)
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 197 - protein, 469 aa, synthetic construct
  REGION 1..469: note = anti-CDH17 CAR
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 880 aa, synthetic construct
  REGION 1..880: note = anti-CDH17.LACO(A4025-CD28)
MALPVTALLLPLALLLHAARPQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 199 - protein, 1024 aa, synthetic construct
  REGION 1..1024: note = anti-CDH17.LACO(A4025-9.3h11)
MALPVTALLLPLALLLHAARPQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 200 - protein, 1057 aa, synthetic construct
  REGION 1..1057: note = anti-CDH17-SR2.LACO(A4025-9.3h11)
MALPVTALLLPLALLLHAARPQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSSGGGGSCPYSNPSLCGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 201 - protein, 1057 aa, synthetic construct
  REGION 1..1057: note = anti-CDH17-R2S.LACO(A4025-9.3h11)
MALPVTALLLPLALLLHAARPGGGGSCPYSNPSLCGGGGSCPYSNPSLCGGGGSQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 202 - protein, 523 aa, synthetic construct
  REGION 1..523: note = anti-CDH17-SR2
MALPVTALLLPLALLLHAARPQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSSGGGGSCPYSNPSLCGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 203 - protein, 523 aa, synthetic construct
  REGION 1..523: note = anti-CDH17-R2S
MALPVTALLLPLALLLHAARPGGGGSCPYSNPSLCGGGGSCPYSNPSLCGGGGSQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 204 - protein, 528 aa, synthetic construct
  REGION 1..528: note = anti-CDH17-RSR
MALPVTALLLPLALLLHAARPGGGGSCPYSNPSLCGGGGSQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSSGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 205 - protein, 9 aa, synthetic construct
  REGION 1..9: note = Rituximab Mimotope
CPYSNPSLC

SEQ ID NO: 206 - protein, 24 aa, synthetic construct
  REGION 1..24: note = Palivizumab Epitope
NSELLSLINDMPITNDQKKLMSNN

SEQ ID NO: 207 - protein, 12 aa, synthetic construct
  REGION 1..12: note = Cetuximab Mimotope-1
CQFDLSTRRLKC

SEQ ID NO: 208 - protein, 12 aa, synthetic construct
  REGION 1..12: note = Cetuximab Mimotope-2
CQYNLSSRALKC

SEQ ID NO: 209 - protein, 12 aa, synthetic construct
  REGION 1..12: note = Cetuximab Mimotope-3
CVWQRWQKSYVC

SEQ ID NO: 210 - protein, 12 aa, synthetic construct
  REGION 1..12: note = Cetuximab Mimotope-4
CMWDRFSRWYKC

SEQ ID NO: 211 - protein, 25 aa, synthetic construct
  REGION 1..25: note = Nivolumab Epitope-5
SFVLNWYRMSPSNQTDKLAAFPEDR

SEQ ID NO: 212 - protein, 19 aa, synthetic construct
  REGION 1..19: note = Nivolumab Epitope-6
SGTYLCGAISLAPKAQIKE

SEQ ID NO: 213 - protein, 16 aa, synthetic construct
  REGION 1..16: note = QBEND-10 Epitope
ELPTQGTFSNVSTNVS

SEQ ID NO: 214 - protein, 12 aa, synthetic construct
  REGION 1..12: note = Alemtuzumab Epitope
GQNDTSQTSSPS

SEQ ID NO: 215 - protein, 32 aa, synthetic construct
  REGION 1..32: note = Furin-GS2-T2A
RAKRGGGGSGGGGSEGRGSLLTCGDVEENPGP

SEQ ID NO: 216 - protein, 11 aa, synthetic construct
  REGION 1..11: note = anti-HER2 H13 LCDR1
RASQSISSYLN

SEQ ID NO: 217 - protein, 7 aa, synthetic construct
  REGION 1..7: note = anti-HER2 H13 LCDR2
AASSLQS

SEQ ID NO: 218 - protein, 9 aa, synthetic construct
  REGION 1..9: note = anti-HER2 H13 LCDR3
QQSYSTPYT

SEQ ID NO: 219 - protein, 5 aa, synthetic construct
  REGION 1..5: note = anti-HER2 H13 HCDR1
SNYMS

SEQ ID NO: 220 - protein, 16 aa, synthetic construct
  REGION 1..16: note = anti-HER2 H13 HCDR2
VIYSGGSTYYADSVKG

SEQ ID NO: 221 - protein, 10 aa, synthetic construct
  REGION 1..10: note = anti-HER2 H13 HCDR3
DFGPTNGFDP

SEQ ID NO: 222 - protein, 107 aa, synthetic construct
  REGION 1..107: note = anti-HER2 H13 VL
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVDIK

SEQ ID NO: 223 - protein, 118 aa, synthetic construct
  REGION 1..118: note = anti-HER2 H13 VH
EVHLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDFGPTNGFDPWGQGTLVTVSS

SEQ ID NO: 224 - protein, 516 aa, synthetic construct
  REGION 1..516: note = anti-HER2 H13 CAR
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVDIKGGGGSGGGGSGGGGSEVHLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDFGPTNGFDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGGGGSGGGGSEGRGSLLTCGDVEENPGPMALPVTALLLPLALLLHAARP

SEQ ID NO: 225 - protein, 13 aa, synthetic construct
  REGION 1..13: note = anti-MESO MH150 LCDR1
SGSSSNIGSNYVY

SEQ ID NO: 226 - protein, 7 aa, synthetic construct
  REGION 1..7: note = anti-MESO MH150 LCDR2
NNNQRPS

SEQ ID NO: 227 - protein, 11 aa, synthetic construct
  REGION 1..11: note = anti-MESO MH150 LCDR3
ATWDDSLSGPV

SEQ ID NO: 228 - protein, 5 aa, synthetic construct
  REGION 1..5: note = anti-MESO MH150 HCDR1
SYAIS

SEQ ID NO: 229 - protein, 17 aa, synthetic construct
  REGION 1..17: note = anti-MESO MH150 HCDR2
GISAYNGNTNYAQKLQG

SEQ ID NO: 230 - protein, 10 aa, synthetic construct
  REGION 1..10: note = anti-MESO MH150 HCDR3
GGLYSGVFDY

SEQ ID NO: 231 - protein, 110 aa, synthetic construct
  REGION 1..110: note = anti-MESO MH150 VL
QPVLTQPPSASGTPGQRVTISCSGSSSNIGSNYVYWYQQLPGTAPKLLLYNNNQRPSGVPDRFSGSKSVTSASLAISGLRSDDEADYYCATWDDSLSGPVFGGGTKLTVL

SEQ ID NO: 232 - protein, 119 aa, synthetic construct
  REGION 1..119: note = anti-MESO MH150 VH
QVQLVQSGAEVKKPGSSVKVSCKASGGTFSSYAISWVRQAPGQGLEWMGGISAYNGNTNYAQKLQGRVTMTTDTSTSTAYMELRSLRSDDTAVYYCARGGLYSGVFDYWGQGTLVTVSS

SEQ ID NO: 233 - protein, 467 aa, synthetic construct
  REGION 1..467: note = anti-MESO MH150 CAR
QPVLTQPPSASGTPGQRVTISCSGSSSNIGSNYVYWYQQLPGTAPKLLLYNNNQRPSGVPDRFSGSKSVTSASLAISGLRSDDEADYYCATWDDSLSGPVFGGGTKLTVLGGGGSGGGGSGGGGSQVQLVQSGAEVKKPGSSVKVSCKASGGTFSSYAISWVRQAPGQGLEWMGGISAYNGNTNYAQKLQGRVTMTTDTSTSTAYMELRSLRSDDTAVYYCARGGLYSGVFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 234 - protein, 14 aa, synthetic construct
  REGION 1..14: note = anti-CDH17 LCDR1
TGTSSDVGGYNYVS

SEQ ID NO: 235 - protein, 7 aa, synthetic construct
  REGION 1..7: note = anti-CDH17 LCDR2
EVSKRPS

SEQ ID NO: 236 - protein, 11 aa, synthetic construct
  REGION 1..11: note = anti-CDH17 LCDR3
SSYAGSNNLRV

SEQ ID NO: 237 - protein, 5 aa, synthetic construct
  REGION 1..5: note = anti-CDH17 HCDR1
NYWIG

SEQ ID NO: 238 - protein, 17 aa, synthetic construct
  REGION 1..17: note = anti-CDH17 HCDR2
MIYPGDSDTRYSPSLRG

SEQ ID NO: 239 - protein, 11 aa, synthetic construct
  REGION 1..11: note = anti-CDH17 HCDR3
LSGSYYSDPDY

SEQ ID NO: 240 - protein, 111 aa, synthetic construct
  REGION 1..111: note = anti-CDH17 VL
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVL

SEQ ID NO: 241 - protein, 120 aa, synthetic construct
  REGION 1..120: note = anti-CDH17 VH
QVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSS

SEQ ID NO: 242 - protein, 246 aa, synthetic construct
  REGION 1..246: note = anti-CDH17 scFv
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLRVFGGGTKLTVLGGGGSGGGGSGGGGSQVQLVQSGAEVKKSGDSLKISCKGSGYFFTNYWIGWVRQMPGKGLEWMGMIYPGDSDTRYSPSLRGQVTISADKSTSTAYLQWSSLKASDTAMYYCARLSGSYYSDPDYWGQGSLVTVSS

SEQ ID NO: 243 - protein, 349 aa, synthetic construct
  REGION 1..349: note = VHH1 CAR
MALPVTALLLPLALLLHAARPGSAAQAAQVQLQESGGGLVQPGGSTRLSCVSSRTFSYYDMGWFRQAPGKEREFVALLSWNGENAEYSDSVMGRFTVSRGNTQNSVNLQMNNLKPEDTGIYYCAVTHGGARSVRSWGQGTQVTVSSAAATSGQAGQSGESKYGPPCPPCPASYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 244 - DNA, 1047 bp, synthetic construct
  misc_feature 1..1047: note = VHH1 CAR DNA
  source 1..1047: mol_type = other DNA
atggccttaccagtgaccgccttgctcctgccgctggccttgctgctccacgccgccaggccgggatccgcggcccaggcggcccaggtgcagctgcaggagtctgggggaggattggtgcagcctgggggctcgacaagactctcctgtgtatcgtcccgcacctttagttattatgacatgggctggttccgccaggctccagggaaggagcgtgagttcgtagcactgcttagttggaatggggaaaatgcagagtattcagactccgtgatgggccgtttcaccgtctcccgagggaatacccagaattcggtgaatctgcaaatgaacaacctgaaacctgaggacacgggcatctattactgcgcagtgacgcacggtggagcgcggtcggttcgttcctggggccaggggacccaggtcaccgtctcctcagcggccgccactagtggccaggccggccagtccggagagtctaagtacggccctccctgccctccttgcccagctagctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 245 - protein, 21 aa, synthetic construct
  REGION 1..21: note = T2A
GSGEGRGSLLTCGDVEENPGP

SEQ ID NO: 246 - protein, 1023 aa, synthetic construct
  REGION 1..1023: note = MSLN-A4025-9.3h12 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSASVGDRVTITCRASESVEYYVTSLMQWYQQKPGKAPKLLIFAASNVESGVPSRFSGSGSGTNFTLTISSVQPEDFATYFCQQSRKVPYTFGGGTKLEIK

SEQ ID NO: 247 - protein, 1023 aa, synthetic construct
  REGION 1..1023: note = MSLN-A4025-9.3h22 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVQLQESGPGLVKPSETLSITCTVSGFSLSDYGVHWVRQPPGKGLEWLGVIWAGGGTNYNSALMSRKTISKDTSKNQVSLKMSSLTAADTAVYYCARDKGYSYYYSMDYWGQGTTVTVSSGGGGSGGGGSGGGGSDIQLTQSPSSLSASVGDRVTITCRASESVEYYVTSLMQWYQQKPGKAPKLLIFAASNVESGVPSRFSGSGSGTNFTLTISSVQPEDFATYFCQQSRKVPYTFGGGTKLEIK

SEQ ID NO: 248 - protein, 1023 aa, synthetic construct
  REGION 1..1023: note = MSLN-A4025-9.3h23 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVQLQESGPGLVKPSETLSITCTVSGFSLSDYGVHWVRQPPGKGLEWLGVIWAGGGTNYNSALMSRKTISKDTSKNQVSLKMSSLTAADTAVYYCARDKGYSYYYSMDYWGQGTTVTVSSGGGGSGGGGSGGGGSDIQLTQSPSSLSASVGDRVTITCRASESVEYYVTSLMQWYQQKPGKAPKLLIFAASNVESGVPSRFSGSGSGTDFTLTISSLQPEDFATYYCQQSRKVPYTFGGGTKLEIK

SEQ ID NO: 249 - protein, 1023 aa, synthetic construct
  REGION 1..1023: note = MSLN-A4025- 9.3h31 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVQLQESGPGLVKPSETLSLTCTVSGFSLSDYGVHWVRQSPGKGLEWIGVIWAGGGTNYNSALKSRKTISKDNSKSQVSLKLSSVTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 250 - protein, 1023 aa, synthetic construct
  REGION 1..1023: note = MSLN-A4025-9.3h32 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVQLQESGPGLVKPSETLSLTCTVSGFSLSDYGVHWVRQSPGKGLEWIGVIWAGGGTNYNSALKSRKTISKDNSKSQVSLKLSSVTAADTAVYYCARDKGYSYYYSMDYWGQGTTVTVSSGGGGSGGGGSGGGGSDIQLTQSPSSLSASVGDRVTITCRASESVEYYVTSLMQWYQQKPGKAPKLLIFAASNVESGVPSRFSGSGSGTNFTLTISSVQPEDFATYFCQQSRKVPYTFGGGTKLEIK

SEQ ID NO: 251 - protein, 1023 aa, synthetic construct
  REGION 1..1023: note = MSLN-A4025-9.3h33 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVQLQESGPGLVKPSETLSLTCTVSGFSLSDYGVHWVRQSPGKGLEWIGVIWAGGGTNYNSALKSRKTISKDNSKSQVSLKLSSVTAADTAVYYCARDKGYSYYYSMDYWGQGTTVTVSSGGGGSGGGGSGGGGSDIQLTQSPSSLSASVGDRVTITCRASESVEYYVTSLMQWYQQKPGKAPKLLIFAASNVESGVPSRFSGSGSGTDFTLTISSLQPEDFATYYCQQSRKVPYTFGGGTKLEIK

SEQ ID NO: 252 - protein, 1023 aa, synthetic construct
  REGION 1..1023: note = MSLN-A4025-9.3h42 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVQLQESGPGLVKPSETLSLTCTVSGFSLSDYGVHWIRQPPGKGLEWIGVIWAGGGTNYNSALKSRVTISVDTSKNQVSLKLSSVTAADTAVYYCARDKGYSYYYSMDYWGQGTTVTVSSGGGGSGGGGSGGGGSDIQLTQSPSSLSASVGDRVTITCRASESVEYYVTSLMQWYQQKPGKAPKLLIFAASNVESGVPSRFSGSGSGTNFTLTISSVQPEDFATYFCQQSRKVPYTFGGGTKLEIK

SEQ ID NO: 253 - protein, 1023 aa, synthetic construct
  REGION 1..1023: note = MSLN-A4025-9.3h43 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVQLQESGPGLVKPSETLSLTCTVSGFSLSDYGVHWIRQPPGKGLEWIGVIWAGGGTNYNSALKSRVTISVDTSKNQVSLKLSSVTAADTAVYYCARDKGYSYYYSMDYWGQGTTVTVSSGGGGSGGGGSGGGGSDIQLTQSPSSLSASVGDRVTITCRASESVEYYVTSLMQWYQQKPGKAPKLLIFAASNVESGVPSRFSGSGSGTDFTLTISSLQPEDFATYYCQQSRKVPYTFGGGTKLEIK

SEQ ID NO: 254 - protein, 1023 aa, synthetic construct
  REGION 1..1023: note = MSLN-A4025-9.3h52 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVQLQESGPGLVKPSETLSLTCTVSGFSISDYGVHWIRQPPGKGLEWIGVIWAGGGTNYNPSLKSRVTISVDTSKNQVSLKLSSVTAADTAVYYCARDKGYSYYYSMDYWGQGTTVTVSSGGGGSGGGGSGGGGSDIQLTQSPSSLSASVGDRVTITCRASESVEYYVTSLMQWYQQKPGKAPKLLIFAASNVESGVPSRFSGSGSGTNFTLTISSVQPEDFATYFCQQSRKVPYTFGGGTKLEIK

SEQ ID NO: 255 - protein, 1023 aa, synthetic construct
  REGION 1..1023: note = MSLN-A4025-9.3 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSQVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSSGGGGSGGGGSGGGGSDIVLTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIK

SEQ ID NO: 256 - protein, 1011 aa, synthetic construct
  REGION 1..1011: note = MSLN-A4025-C65 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSMTQSPSSLSASVGDRVTIACTASQILTVYLIIGSNYVYWYQQLPGTAPKLLLYNAWVDGNSGVPDRFSGSKSVTSASLAISDVRNIVAYFGQGTKVEIKGGGGSGGGGSGGGGSEVQLVESGGGVVQPGRSLRLSCAASGFTFSSYFMHWVRQAPGKGLEWVAVISYDGSNKHYADSVKGRFTISRDNSKNTLYLQMNSLKPEDTAVYYCAREYWNQRDGMDVWGQGTTVTVSS

SEQ ID NO: 257 - protein, 1016 aa, synthetic construct
  REGION 1..1016: note = MSLN-A4025-C58 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSMTQSPSSLSASVGDRVTIACRASQGVSDYLAWYQQKPGTAPKLLIYGASTLQSGVPSRFSGSGSGTDFTLTISDLQPEDFATYYCLQLNTYPYTFGQGTKVEIKGGGGSGGGGSGGGGSEVQLVESGGGVVQPGRSLRLSCAASGFTFSSYFMHWVRQAPGKGLEWVAVISYDGSNKHYADSVKGRFTISRDNSKNTLYLQMNSLKPEDTAVYYCAREYWNQRDGMDVWGQGTTVTVSS

SEQ ID NO: 258 - protein, 1021 aa, synthetic construct
  REGION 1..1021: note = MSLN-A4025-28.3 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSGGGGSGGGGSGGGGSDIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKRT

SEQ ID NO: 259 - protein, 1020 aa, synthetic construct
  REGION 1..1019: note = MSLN-A4025-h28.3 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSVQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSAGGGGSGGGGSGGGGSDIQMTQSPSSLSASVGDRVTITCKTNENIYSNLAWYQQKDGKSPQLLIYAATHLVEGVPSRFSGSGSGTQYSLTISSLQPEDFGNYYCQHFWGTPXTFGGGTKLEIKR

SEQ ID NO: 260 - protein, 1043 aa, synthetic construct
  REGION 1..1043: note = MSLN-A4025-1412 Amino acid sequence, soluble
MALPVTALLLPLALLLHAARPAIRMTQSPSSLSASVGDRVTITCRASQGISNSLAWYQQKPGKAPKLLLYAASRLESGVPSRFSGSGSGTDYTLTISSLQPEDFATYYCQQYYSTPFTFGPGTKLEIKGGGGSGGGGSGGGGSQVQLVQSGAEVKKPGSSVKVSCKTSADTFSANTINWVRQAPGHGLEWMGRIIPSLNIRDYAQEFQGRVTFTADESTNTVYMQLTSLRSEDTAVYYCARDPGSTWSPNQFFPHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPQSALTQPASVSGSPGQSITVSCTGTRSDIGDYNYVSWYQQHPGKAPKLIIYDVSSRPSGVSNRYSGSKSGNTASLTISGLQAEDEADYYCSSYTSSSTLVFGGGTKVTVLGGGGSGGGGSGGGGSQVQLVQSGGGLVQPGGSLRLSCAASGFTFSSNWMSWVRQAPGKGLEWVANINQDGSEKYYVDSVKGRFTISRDNAKNSLYLQLNSLRAEDTAEYYCARGGYRYAYWGQGTLVTVSSGGGGSALPVTALLLPLALLLHAARPQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSVEGGSGGSGGSGGSGGVMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 261 - protein, 920 aa, synthetic construct
  REGION 1..920: note = CD27z-9-9.3
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSPRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGGGGSGGGGSEGRGSLLTCGDVEENPGPMALPVTALLLPLALLLHAARPQAVLTQPPSASGAPGQRVSISCSGGSSNIGSHTVNWYQQLPGAAPRLLMFSNYQRPSGVPDRFSASKSGTSASLAISGLRSEDEADYYCAAWDGTLKGYVFGTGTKVTVLGGGGSGGGGSGGGGSEVQLVESGGGVVLPGRSLRLSCAASGFTLRSYGMHWVRQATGKGLEWVSAIGTAGDTYYPGSVKGRFTISRENAKNSLYLQMNSLRAGDTAVYYCARADRLGGYGMDVWGQGTTVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 262 - protein, 924 aa, synthetic construct
  REGION 1..924: note = CD27z-11-9.3
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSPRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGGGGSGGGGSEGRGSLLTCGDVEENPGPMALPVTALLLPLALLLHAARPEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVEIKGGGGSGGGGSGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRSTIFGVVRRAGMDVWGQGTTVTVSSGGGGSQVKLQESGPGLVKPSETLSITCTVSGFSLSDYGVHWVRQSPGKGLEWLGVIWAGGGTNYNSALMSRKTISKDNSKSQVFLKMSSLTAADTAVYYCARDKGYSYYYSMDYWGQGTTVTVSSGGGGSGGGGSGGGGSDIQLTQSPSSLSVSVGDRVTISCRASESVEYYVTSLMQWYQQKPGQAPKLLIFAASNVESGVPSRFSGSGSGTNFTLTISSVQEEDFAMYFCQQSRKVPYTFGGGTKLEIK

SEQ ID NO: 263 - protein, 375 aa, synthetic construct
  REGION 1..375: note = A4D11C2828
MALPVTALLLPLALLLHAARPQLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASGGGGSGGGGSGGGGSAIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRTEIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 264 - protein, 378 aa, synthetic construct
  REGION 1..378: note = A119C2828
MALPVTALLLPLALLLHAARPQVQLQQSGPGLVKPSQTLSLTCAISGDSVSSNSATWNWIRQSPSRDLEWLGRTYYRSKWYRDYVGSVKSRIIINPDTSNNQFSLQLNSVTPEDTAIYYCTRAQWLGGDYPYYYSMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIKRTAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

(19) United States
(12) Patent Application Publication
(10) Pub. No.: US 2026/0256913 A1
(43) Pub. Date: Sep. 3, 2026

(54) CD40-TARGETTING ANTIBODIES AND USES THEREOF

(71) Applicant: UTC TYHERAPEUTICS (SHANGNAI) CO., LTD., Shanghai (CN)

(72) Inventors: Yangbing ZHAO, Shanghai (CN); Xiaojun LIU, Shanghai (CN); Gengzhen Zhu, Shanghai (CN)

(73) Assignee: UTC TYHERAPEUTICS (SHANGNAI) CO., LTD., Shanghai (CN)

(21) Appl. No.: 19/665,527

(22) Filed: May 1, 2026

Related U.S. Application Data
(63) Continuation of application No. PCT/CN2024/129735, filed on Nov. 4, 2024.

(30) Foreign Application Priority Data
Nov. 3, 2023 (WO) PCT/CN2023/129560
Mar. 21, 2024 (WO) PCT/CN2024/082974
May 17, 2024 (WO) PCT/CN2024/093913
Aug. 29, 2024 (WO) PCT/CN2024/115328
Aug. 29, 2024 (WO) PCT/CN2024/115417

(51) Int. Cl. A61K 40/42; A61K 40/11; A61K 40/15; A61K 40/33; C07K 16/28

(52) U.S. Cl. CPC A61K 40/421; A61K 40/11; A61K 40/15; A61K 40/33; C07K 16/2818; C07K 16/2878; C07K 2317/622

(57) ABSTRACT

Disclosed herein are CD40-targetting antibodies and uses thereof. Disclosed herein are novel antigen-binding proteins that specifically bind CD40 and antigen-binding fragments thereof. Disclosed herein are also fusion proteins comprising a first domain that activates an antigen-presenting cell (APC) (e.g., a dendritic cell) and a second domain that activates an immune effector cell (e.g., a T cell), wherein the first domain comprises antigen-binding proteins that specifically bind CD40 or antigen-binding fragments thereof disclosed herein.

### CROSS REFERENCE TO RELATED APPLICATIONS

This application is a Rule 53(b) Continuation of International Application No. PCT/CN2024/129735 filed on Nov. 4, 2024, claiming priority based on International Application No. PCT/CN2023/129560 filed on Nov. 3, 2023, International Application No. PCT/CN2024/115417 filed on Aug. 29, 2024, International Application No. PCT/CN2024/082974 filed on Mar. 21, 2024, International Application No. PCT/CN2024/093913 filed on May 17, 2024, and International Application No. PCT/CN2024/115328 filed on Aug. 29, 2024, the respective disclosures of all of the above of which are incorporated herein by reference in their entirety.

### FIELD

### Incorporation by Reference of Sequence Listing

The instant application contains a Sequence Listing which has been filed electronically in xml format and is hereby incorporated by reference in its entirety. Said xml copy, created on May 1, 2026, is named Q319909_sequencelistingasfiled.xml and is 390,285 bytes in size.

The present invention relates to molecular biology and immuno-oncology. Provided herein include anti-CD40-antibodies and uses thereof in treating tumors or cancers.

### BACKGROUND

T cells can be engineered to express T cell receptors (TCRs) (Morgan R A et al., Science (2006) 314(5796):126-129; Robbins P F et al., J Clin Oncol (2011) 29(7):917-924; Rapoport A P et al., Nature Medicine (2015) 21(8):914-921) or chimeric antigen receptor (CAR) (Kochenderfer I N et al., Blood (2010) 116(20):4099-4102; Kalos M et al., Science Translational Medicine (2011) 3(95):95ra73) that recognize disease-specific antigens for the treatment of cancers and other diseases. Although T cells engineered with CARs specific to the B cell markers, such as CD19, showed dramatic clinical responses in hematological malignancies, effective immunotherapy in solid cancers has proven to be challenging, mainly due to the immune escape caused by complex, dynamic tumor microenvironment (TME) that induces T cell hypofunction and exhaustion and limits the antitumor immune response (Anderson K G et al, Cancer Cell (2017) 31(3):311-325). Thus, strategies to circumvent suppressive pathways without causing systemic toxicities represent unmet need.

Human cancers and chronic infections can be treated with agents that modulate the patient's immune response to malignant or infected cells. Anti-CD40 antibodies have been tried for treating cancer because they can enhance immune responses. See, e.g., Kirkwood et al. (2012) CA Cancer J. Clin. 62:309; Vanderheide & Glennie (2013) Clin. Cancer Res. 19: 1035. The need exists for improved agonistic anti-human CD40 antibodies for treatment of cancer and chronic infections in human subjects.

The anti-CD40 antibodies, fusion proteins, and related compositions and methods provided herein meet these needs and provide other relative advantages.

### SUMMARY

The present disclosure provides antibodies or antigen-binding fragments thereof that specifically bind CD40 (e.g., human CD40), comprising:
a) a heavy chain variable region (VH) comprising
   i. a heavy chain CDR1 (HCDR1) comprising an amino acid sequence as set forth in SEQ ID NO: 2, a heavy chain CDR2 (HCDR2) comprising an amino acid sequence as set forth in SEQ ID NO: 3, a heavy chain CDR3 (HCDR3) comprising an amino acid sequence as set forth in SEQ ID NO: 4;
   ii. a heavy chain CDR1 (HCDR1) comprising an amino acid sequence as set forth in SEQ ID NO: 11, a heavy chain CDR2 (HCDR2) comprising an amino acid sequence as set forth in SEQ ID NO: 12, a heavy chain CDR3 (HCDR3) comprising an amino acid sequence as set forth in SEQ ID NO: 13;
   iii. a heavy chain CDR1 (HCDR1) comprising an amino acid sequence as set forth in SEQ ID NO: 2, a heavy chain CDR2 (HCDR2) comprising an amino acid sequence as set forth in SEQ ID NO: 3, a heavy chain CDR3 (HCDR3) comprising an amino acid sequence as set forth in SEQ ID NO: 20;
   iv. a heavy chain CDR1 (HCDR1) comprising an amino acid sequence as set forth in SEQ ID NO: 27, a heavy chain CDR2 (HCDR2) comprising an amino acid sequence as set forth in SEQ ID NO: 28, a heavy chain CDR3 (HCDR3) comprising an amino acid sequence as set forth in SEQ ID NO: 29;
   v. a heavy chain CDR1 (HCDR1) comprising an amino acid sequence as set forth in SEQ ID NO: 36, a heavy chain CDR2 (HCDR2) comprising an amino acid sequence as set forth in SEQ ID NO: 37, a heavy chain CDR3 (HCDR3) comprising an amino acid sequence as set forth in SEQ ID NO: 38;
   vi. a heavy chain CDR1 (HCDR1) comprising an amino acid sequence as set forth in SEQ ID NO: 45, a heavy chain CDR2 (HCDR2) comprising an amino acid sequence as set forth in SEQ ID NO: 46, a heavy chain CDR3 (HCDR3) comprising an amino acid sequence as set forth in SEQ ID NO: 47;
   vii. a heavy chain CDR1 (HCDR1) comprising an amino acid sequence as set forth in SEQ ID NO: 54, a heavy chain CDR2 (HCDR2) comprising an amino acid sequence as set forth in SEQ ID NO: 55, a heavy chain CDR3 (HCDR3) comprising an amino acid sequence as set forth in SEQ ID NO: 56;
   viii. a heavy chain CDR1 (HCDR1) comprising an amino acid sequence as set forth in SEQ ID NO: 62, a heavy chain CDR2 (HCDR2) comprising an amino acid sequence as set forth in SEQ ID NO: 63, a heavy chain CDR3 (HCDR3) comprising an amino acid sequence as set forth in SEQ ID NO: 64;
   or a variant thereof having up to about 3 amino acid substitutions, additions, and/or deletions in the HCDRs; and/or
b) a light chain variable region (VL) comprising
   i. a light chain CDR1 (LCDR1) comprising an amino acid sequence as set forth in SEQ ID NO: 6, a light chain CDR2 (LCDR2) comprising an amino acid sequence as set forth in SEQ ID NO: 7, a light chain CDR3 (LCDR3) comprising an amino acid sequence as set forth in SEQ ID NO: 8;
   ii. a light chain CDR1 (LCDR1) comprising an amino acid sequence as set forth in SEQ ID NO: 15, a light chain CDR2 (LCDR2) comprising an amino acid sequence as set forth in SEQ ID NO: 16, a light chain CDR3 (LCDR3) comprising an amino acid sequence as set forth in SEQ ID NO: 17;
   iii. a light chain CDR1 (LCDR1) comprising an amino acid sequence as set forth in SEQ ID NO: 22, a light chain CDR2 (LCDR2) comprising an amino acid sequence as set forth in SEQ ID NO: 23, a light chain CDR3 (LCDR3) comprising an amino acid sequence as set forth in SEQ ID NO: 24;
   iv. a light chain CDR1 (LCDR1) comprising an amino acid sequence as set forth in SEQ ID NO: 31, a light chain CDR2 (LCDR2) comprising an amino acid sequence as set forth in SEQ ID NO: 32, a light chain CDR3 (LCDR3) comprising an amino acid sequence as set forth in SEQ ID NO: 33;
   v. a light chain CDR1 (LCDR1) comprising an amino acid sequence as set forth in SEQ ID NO: 40, a light chain CDR2 (LCDR2) comprising an amino acid sequence as set forth in SEQ ID NO: 41, a light chain CDR3 (LCDR3) comprising an amino acid sequence as set forth in SEQ ID NO: 42;
   vi. a light chain CDR1 (LCDR1) comprising an amino acid sequence as set forth in SEQ ID NO: 49, a light chain CDR2 (LCDR2) comprising an amino acid sequence as set forth in SEQ ID NO: 50, a light chain CDR3 (LCDR3) comprising an amino acid sequence as set forth in SEQ ID NO: 51;
   vii. a light chain CDR1 (LCDR1) comprising an amino acid sequence as set forth in SEQ ID NO: 58, a light chain CDR2 (LCDR2) comprising an amino acid sequence as set forth in SEQ ID NO: 41, a light chain CDR3 (LCDR3) comprising an amino acid sequence as set forth in SEQ ID NO: 59;
   vii. a light chain CDR1 (LCDR1) comprising an amino acid sequence as set forth in SEQ ID NO: 66, a light chain CDR2 (LCDR2) comprising an amino acid sequence as set forth in SEQ ID NO: 67, a light chain CDR3 (LCDR3) comprising an amino acid sequence as set forth in SEQ ID NO: 68;
   ix. a light chain CDR1 (LCDR1) comprising an amino acid sequence as set forth in SEQ ID NO: 40, a light chain CDR2 (LCDR2) comprising an amino acid sequence as set forth in SEQ ID NO: 41, a light chain CDR3 (LCDR3) comprising an amino acid sequence as set forth in SEQ ID NO: 42; or a variant thereof having up to about 3 amino acid substitutions, additions, and/or deletions in the LCDRs.

In some embodiments of the antibodies and antigen-binding fragments thereof provided herein, 1) the HCDR1, HCDR2 and HCDR3 comprise the amino acid sequence as set forth in SEQ ID NOs: 2, 3 and 4, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 6, 7, and 8, respectively; or a variant thereof having up to about 3 amino acid substitutions, additions, and/or deletions in the LCDRs; 2) the HCDR1, HCDR2 and HCDR3 comprise the amino acid sequence as set forth in SEQ ID NOs: 11, 12 and 13, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 15, 16, and 17, respectively; or a variant thereof having up to about 3 amino acid substitutions, additions, and/or deletions in the LCDRs; 3) the HCDR1, HCDR2 and HCDR3 comprise the amino acid sequence as set forth in SEQ ID NOs: 2, 3 and 20, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 22, 23, and 24, respectively; or a variant thereof having up to about 3 amino acid substitutions, additions, and/or deletions in the LCDRs; 4) the HCDR1, HCDR2 and HCDR3 comprise the amino acid sequence as set forth in SEQ ID NOs: 27, 28 and 29, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 31, 32, and 33, respectively; or a variant thereof having up to about 3 amino acid substitutions, additions, and/or deletions in the LCDRs; 5) the HCDR1, HCDR2 and HCDR3 comprise the amino acid sequence as set forth in SEQ ID NOs: 36, 37 and 38, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 40, 41, and 42, respectively; or a variant thereof having up to about 3 amino acid substitutions, additions, and/or deletions in the LCDRs; 6) the HCDR1, HCDR2 and HCDR3 comprise the amino acid sequence as set forth in SEQ ID NOs: 45, 46 and 47, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 49, 50, and 51, respectively; or a variant thereof having up to about 3 amino acid substitutions, additions, and/or deletions in the LCDRs; 7) the HCDR1, HCDR2 and HCDR3 comprise the amino acid sequence as set forth in SEQ ID NOs: 54, 55 and 56, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 58, 41, and 59, respectively; or a variant thereof having up to about 3 amino acid substitutions, additions, and/or deletions in the LCDRs; 8) the HCDR1, HCDR2 and HCDR3 comprise the amino acid sequence as set forth in SEQ ID NOs: 62, 63 and 64, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 66, 67, and 68, respectively; or a variant thereof having up to about 3 amino acid substitutions, additions, and/or deletions in the LCDRs; or 9) the HCDR1, HCDR2 and HCDR3 comprise the amino acid sequence as set forth in SEQ ID NOs: 45, 46 and 47, respectively; or a variant thereof having up to about 3 amino acid substitutions, additions, and/or deletions in the HCDRs; and the LCDR1, LCDR2 and LCDR3 comprise the amino acid sequence as set forth in SEQ ID NOs: 71, 72, and 51, respectively; or a variant thereof having up to about 3 amino acid substitutions, additions, and/or deletions in the LCDRs.

In some embodiments of the antibodies and antigen-binding fragments thereof provided herein, a) the VH comprising at least 85%, at least 90%, at least 95%, at least 98%, or 100% sequence identity to an amino acid sequence as set forth in any one of SEQ ID NOs: 1, 10, 19, 26, 35, 44, 53 and 61; and/or b) the VL comprising at least 85%, at least 90%, at least 95%, at least 98%, or 100% sequence identity to an amino acid sequence as set forth in any one of SEQ ID NOs: 5, 14, 21, 30, 39, 48, 57, 65 and 70.

In some embodiments of the antibodies and antigen-binding fragments thereof provided herein, 1) the VH comprising an amino acid sequence as set forth in SEQ ID NO: 1, and the VL comprising an amino acid sequence as set forth in SEQ ID NO: 5; 2) the VH comprising an amino acid sequence as set forth in SEQ ID NO: 10, and the VL comprising an amino acid sequence as set forth in SEQ ID NO: 14; 3) the VH comprising an amino acid sequence as set forth in SEQ ID NO: 19, and the VL comprising an amino acid sequence as set forth in SEQ ID NO: 21; 4) the VH comprising an amino acid sequence as set forth in SEQ ID NO: 26, and the VL comprising an amino acid sequence as set forth in SEQ ID NO: 30; 5) the VH comprising an amino acid sequence as set forth in SEQ ID NO: 35, and the VL comprising an amino acid sequence as set forth in SEQ ID NO: 39; 6) the VH comprising an amino acid sequence as set forth in SEQ ID NO: 44, and the VL comprising an amino acid sequence as set forth in SEQ ID NO: 48; 7) the VH comprising an amino acid sequence as set forth in SEQ ID NO: 53, and the VL comprising an amino acid sequence as set forth in SEQ ID NO: 57; 8) the VH comprising an amino acid sequence as set forth in SEQ ID NO: 61, and the VL comprising an amino acid sequence as set forth in SEQ ID NO: 65; or 9) the VH comprising an amino acid sequence as set forth in SEQ ID NO: 44, and the VL comprising an amino acid sequence as set forth in SEQ ID NO: 70.

In some embodiments of the antibodies and antigen-binding fragments thereof provided herein, the antigen-binding protein is an antibody or an antigen-binding fragment.

In some embodiments of the antibodies and antigen-binding fragments thereof provided herein, the antigen-binding protein comprises a Fab, a Fab′, a F(ab′)2, a Fv, a scFv, a (scFv)2, a single domain antibody (sdAb), and/or a heavy chain antibody (HCAb).

In some embodiments of the antibodies and antigen-binding fragments thereof provided herein, the antigen-binding protein comprises a scFv.

In some embodiments of the antibodies and antigen-binding fragments thereof provided herein, the antigen-binding protein comprises a scFv, and the scFv comprises an amino acid sequence as set forth in any one of SEQ ID NOs: 9, 18, 25, 34, 43, 52, 60, 69, and 73.

In some embodiments of the antibodies and antigen-binding fragments thereof provided herein, the antigen-binding protein comprises a chimeric antibody or antigen-binding fragment, a humanized antibody or antigen-binding fragment, or a human antibody or antigen-binding fragment.

In some embodiments of the antibodies and antigen-binding fragments thereof provided herein, the antigen-binding protein comprises an IgG1 antibody, an IgG2 antibody, an IgG3 antibody, and an IgG4 antibody.

In some embodiments of the antibodies and antigen-binding fragments thereof provided herein, the antigen-binding protein comprises a bispecific antibody or a multispecific antibody.

Also provided herein are polypeptides comprising the antigen-binding protein provided herein.

Also provided herein are polynucleotides encoding the antigen-binding protein provided herein and/or the polypeptide provided herein.

Also provided herein are vectors comprising the polynucleotide provided herein.

Also provided herein are cells comprising the antigen-binding protein provided herein, the polypeptide provided herein, the polynucleotide provided herein and/or the vector provided herein.

Also provided herein are pharmaceutical compositions comprising the antigen-binding protein provided herein, the polypeptide provided herein, the polynucleotide provided herein and/or the vector provided herein, and a pharmaceutically acceptable excipient.

Also provided herein are methods for preventing and/or treating a disease or a disorder, comprising administering the antigen-binding protein provided herein, the polypeptide provided herein, the polynucleotide provided herein, the vector provided herein and/or the pharmaceutical composition provided herein.

In some embodiments of the methods provided herein, the disease or the disorder comprises a tumor or a cancer.

Also provided herein are fusion proteins comprising a first domain and a second domain, wherein (i) the first domain comprises the antibody or antigen-binding fragment provided herein; and (ii) the second domain activates an immune effector cell and comprises (a) a co-stimulatory receptor of the immune effector cell, or a functional fragment thereof, (b) a co-stimulatory ligand of the immune effector cell, or a receptor-binding fragment thereof, or (c) an antibody or antigen-binding fragment that binds a co-stimulatory receptor of the immune effector cell, or an antigen-binding fragment thereof.

In some embodiments of the fusion proteins, the second domain comprises a cytoplasmic domain of the co-stimulatory receptor. In some embodiments of the fusion proteins, the second domain further comprises the transmembrane domain of the co-stimulatory receptor.

In some embodiments of the fusion proteins, the co-stimulatory receptor comprises CD28, 4-1BB, ICOS, CD27, OX40, DAP10, 2B4, CD30, CD2, LIGHT, GITR, TLR, DR3, and/or CD43.

In some embodiments of the fusion proteins, the co-stimulatory receptor is CD28 or 4-1BB.

In some embodiments of the fusion proteins, the second domain comprises the amino acid sequence as set forth in SEQ ID NO: 92.

In some embodiments of the fusion proteins, the second domain comprises a co-stimulatory ligand of the immune effector cell, or a receptor-binding fragment thereof, the co-stimulatory ligand comprises CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R, and/or CD44.

In some embodiments of the fusion proteins, the second domain comprises an antibody or an antigen-binding fragment that binds the co-stimulatory receptor, or an antigen-binding fragment thereof, and the co-stimulatory receptor comprises CD28, 4-1BB, ICOS, CD27, OX40, DAP10, 2B4, CD30, CD2, LIGHT, GITR, TLR, DR3, and/or CD43.

In some embodiments of the fusion proteins, the co-stimulatory receptor is CD28.

In some embodiments of the fusion proteins, the second domain comprises an antibody or an antigen-binding fragment that specifically binds CD28 and comprises a heavy chain variable region (VH) and/or a light chain variable region (VL), wherein the VH comprises a heavy chain CDR1 (HCDR1) comprising the amino acid sequence as set forth in SEQ ID NO: 177, a heavy chain CDR2 (HCDR2) comprising the amino acid sequence as set forth in SEQ ID NO: 178, a heavy chain CDR3 (HCDR3) comprising the amino acid sequence as set forth in SEQ ID NO: 179, or a variant thereof having up to about 3 amino acid substitutions, additions, and/or deletions in the HCDRs; wherein the VL comprises a light chain CDR1 (LCDR1) comprising the amino acid sequence as set forth in SEQ ID NO: 180, a light chain CDR2 (LCDR2) comprising the amino acid sequence as set forth in SEQ ID NO: 181, a light chain CDR3 (LCDR3) comprising the amino acid sequence as set forth in SEQ ID NO: 182, or a variant thereof having up to about 3 amino acid substitutions, additions, and/or deletions in the LCDRs.

In some embodiments of the fusion proteins, the VH of the second domain comprising at least 85%, at least 90%, at least 95%, at least 98%, or 100% sequence identity to the amino acid sequence as set forth in any of SEQ ID NO: 183, and the VL of the second domain comprising at least 85%, at least 90%, at least 95%, at least 98%, or 100% sequence identity to the amino acid sequence as set forth in any of SEQ ID NO: 184.

In some embodiments of the fusion proteins, the VH of the second domain comprising the amino acid sequence as set forth in any of SEQ ID NO: 183, and the VL of the second domain comprising the amino acid sequence as set forth in any of SEQ ID NO: 184.

In some embodiments of the fusion proteins, the first domain and/or the second domain comprises a scFv.

In some embodiments of the fusion proteins, the scFv comprises a VH and a VL linked by a peptide linker.

In some embodiments of the fusion proteins, the linker comprises the amino acid sequence as set forth in SEQ ID NO: 97.

In some embodiments of the fusion proteins, the first domain comprises the amino acid sequence as set forth in SEQ ID NO: 9, 18, 25, 34, 43, 52, 60, 69, 73, 82 or 91, and the second domain comprises the amino acid sequence as set forth in SEQ ID NO: 96 or SEQ ID NO: 185.

In some embodiments of the fusion proteins, the N-terminus of the first domain is linked to the C-terminus of the second domain.

In some embodiments of the fusion proteins, the N-terminus of the second domain is linked to the C-terminus of the first domain.

In some embodiments of the fusion proteins, the first domain and the second domain are linked via a linker.

In some embodiments of the fusion proteins, the linker comprises an amino acid sequence as set forth in any one of SEQ ID NOs: 97-100.

In some embodiments of the fusion proteins, the fusion protein comprises an amino acid sequence that is at least 85%, 90%, 95%, 98%, or 99% identical to the amino acid sequences as set forth in any one of SEQ ID NO: 155-165 or SEQ ID NO: 186.

In some embodiments of the fusion proteins, the fusion protein comprises a bispecific antibody or an antigen-binding fragment thereof.

In some embodiments of the fusion proteins, wherein the fusion protein further comprises one or more Fc domain.

Also provided herein are polynucleotides encoding the fusion protein provided herein.

Also provided herein are vectors comprising the polynucleotide provided herein.

In some embodiments of the vectors, the vector further comprises a polynucleotide encoding a chimeric antigen receptor (CAR), a T cell receptor (TCR) or a Bi-specific T-cell engager (BiTE), wherein the CAR, TCR or BiTE binds a tumor antigen or a viral antigen.

In some embodiments of the vectors, the tumor antigen comprises CD70, HER2, NY-ESO-1, CD19, CD20, CD22, PSMA, c-Met, GPC3, IL13ra2, EGFR, CD123, CD7, GD2, PSCA, EBV16-E7, H3.3, EGFRvIII, BCMA, Mesothelin, GPRC5D, GCC, GUCY2C, Claudin 18.2, ROR1, B7H3, DLL3 and/or CDH17.

In some embodiments of the vectors, the viral antigen comprises HPV, EBV, and/or HIV.

In some embodiments of the vectors, the polynucleotide encoding the fusion protein is linked with the polynucleotide encoding the chimeric antigen receptor (CAR), the T cell receptor (TCR) or the Bi-specific T-cell engager (BiTE) directly or indirectly.

In some embodiments of the vectors, the polynucleotide encoding the fusion protein is linked with the polynucleotide encoding the chimeric antigen receptor (CAR), the T cell receptor (TCR) or the Bi-specific T-cell engager (BiTE) by a linker.

In some embodiments of the vectors, the linker comprises a polynucleotide encoding F2A peptide.

In some embodiments of the vectors, the vector is a viral vector.

Also provided herein are pharmaceutical compositions comprising the fusion protein provided herein, the polynucleotide provided herein, and/or the vector provided herein, and a pharmaceutically acceptable excipient.

Also provided herein are methods for preventing and/or treating a disease or a disorder, comprising administering the fusion protein provided herein, the polynucleotide provided herein, the vector provided herein, and/or the pharmaceutical composition provided herein to a subject.

In some embodiments of the methods, the fusion protein is used in combination with an immune effector cell.

In some embodiments of the methods, the immune effector cell comprises a CAR-T cell, a TCR-T cell, a TIL, a CIK, a LAK, and/or a MIL.

Also provided herein are cells that comprise the fusion protein provided herein, the polynucleotide provided herein, and/or the vector provided herein.

In some embodiments of the cells, the cell is genetically engineered and recombinantly expresses the fusion protein provided herein.

In some embodiments of the cells, the cell comprises an immune effector cell.

In some embodiments of the cells, the immune effector cell comprises a T cell, an NK cell, an NKT cell, a macrophage, a neutrophil, and a granulocyte.

In some embodiments of the cells, the cell further comprises a chimeric antigen receptor (CAR), a T cell receptor (TCR) or a Bi-specific T-cell engager (BiTE), and/or a polynucleotide that encodes a CAR, a TCR, or a BiTE, wherein the CAR, TCR or BiTE binds a tumor antigen or a viral antigen.

In some embodiments of the cells, the viral antigen comprises HPV, EBV, and/or HIV.

In some embodiments of the cells, the tumor antigen comprises CD70, HER2, NY-ESO-1, CD19, CD20, CD22, PSMA, c-Met, GPC3, IL13ra2, EGFR, CD123, CD7, GD2, PSCA, EBV16-E7, H3.3, EGFRvIII, BCMA, Mesothelin, GPRC5D, GCC, GUCY2C, Claudin 18.2, ROR1, B7H3, DLL3 and/or CDH17.

In some embodiments of the cells, the CAR comprises an antigen-binding domain, a transmembrane domain and a cytoplasmic domain.

In some embodiments of the cells, the cell is derived from a cell isolated from peripheral blood or bone marrow.

In some embodiments of the cells, the cell is derived from a cell differentiated in vitro from a stem or progenitor cell selected from the group consisting of a T cell progenitor cell, a hematopoietic stem and progenitor cell, a hematopoietic multipotent progenitor cell, an embryonic stem cell, and an induced pluripotent cell.

In some embodiments of the cells, the cell is a T cell.

In some embodiments of the cells, the cell comprises a cytotoxic T cell, a helper T cell, or a gamma delta T, a CD4+/CD8+ double positive T cell, a CD4+ T cell, a CD8+ T cell, a CD4/CD8 double negative T cell, a CD3+ T cell, a naïve T cell, an effector T cell, a cytotoxic T cell, a helper T cell, a memory T cell, a regulator T cell, a Th0 cell, a Th1 cell, a Th2 cell, a Th3 (Treg) cell, a Th9 cell, a Th17 cell, a Thap helper cell, a Tfh cell, a stem memory TSCM cell, a central memory TCM cell, an effector memory TEM cell, an effector memory TEMRA cell, or a gamma delta T cell.

In some embodiments of the cells, the cell comprises a population of the cells, and the cells are derived from cells isolated from peripheral blood mononuclear cells (PBMC), peripheral blood leukocytes (PBL), tumor infiltrating lymphocytes (TIL), cytokine-induced killer cells (CIK), lymphokine-activated killer cells (LAK), or marrow infiltrate lymphocytes (MILs).

Also provided herein are methods for producing the cell provided herein.

In some embodiments, the methods comprising transferring the polynucleotide provided herein into the cell.

In some embodiments of the methods, the polynucleotide is transferred by electroporation, viral transduction, using a transposon system, and/or using a gene-editing system.

In some embodiments of the methods, the gene-editing system comprises a CRISPR-Cas system, a ZFN system, and/or a TALEN system.

Also provided herein are pharmaceutical compositions comprising the cell provided herein, and a pharmaceutically acceptable excipient.

Also provided herein are methods for preventing and/or treating a disease or a disorder, comprising administering the cell provided herein and/or the pharmaceutical composition provided herein to a subject.

In some embodiments of the methods, the cell and/or the pharmaceutical composition reduces cancer-induced immunosuppression.

In some embodiments, the method comprises administering a cell therapy to the subject.

In some embodiments of the methods, the cell therapy is selected from the group consisting of a CAR T therapy, a TCRT therapy, a TIL therapy, a CIK therapy, a LAK therapy, and a MIL therapy.

In some embodiments of the methods, the subject is a human.

In some embodiments of the methods, the disease or the disorder comprises a tumor and/or cancer.

In some embodiments of the methods, the disease or the disorder comprises a solid tumor and/or a hematological cancer.

In some embodiments of the methods, the disease or the disorder comprises a CD70-expressing cancer, a HER2-expressing cancer, a NY-ESO-1-expressing cancer, a CD19-expressing cancer, a CD20-expressing cancer, a CD22-expressing cancer, a PSMA-expressing cancer, a c-Met-expressing cancer, a GPC3-expressing cancer, a IL13ra2-expressing cancer, a EGFR-expressing cancer, a CD123-expressing cancer, a CD7-expressing cancer, a GD2-expressing cancer, a PSCA-expressing cancer, a EBV16-E7-expressing cancer, a H3.3-expressing cancer, a EGFRvIII-expressing cancer, a BCMA-expressing cancer, a Mesothelin-expressing cancer, a GPRC5D-expressing cancer, a GCC-expressing cancer, a GUCY2C-expressing cancer, a Claudin 18.2-expressing cancer, a ROR1-expressing cancer, a B7H3-expressing cancer, a DLL3-expressing cancer and/or a CDH17-expressing cancer.

In some embodiments, the method further comprising administering an additional therapy to the subject.

Also provided herein are vaccine adjuvants comprising the antigen-binding protein that specifically binds CD40 provided herein.

### BRIEF DESCRIPTION OF THE DRAWINGS

FIG. 1 provides the flow cytometric staining results of constructing a CD40 overexpressing MOLM14 stably transfected cell line.

FIG. 2 provides the cell-phage ELISA screening results. The value is the difference between the MOLM14-CD40 reading minus the background cell MOLM14 reading. Clones marked with a gray background (clone numbers 1-46) were selected as candidate clones for subsequent PCR-amplified scFv sequences for sequencing.

FIG. 3 provides the schematic diagram of pDA.anti-CD40 scFv.CD28 vector.

FIG. 4 provides the flow cytometry results of CD40-Fc recombinant protein and anti-human IgG-Fc antibody staining after electroporation of 11 candidate anti-CD40 scFv.CD28 mRNA into T cells.

FIG. 5 provides the schematic diagram of pUTCK.anti-CD40 scFv.BBZ vector.

FIG. 6 provides the detection results of DC activation marker CD80 after co-incubation of different CAR-T cells under the conditions of DC: T=1:2.5 and 1:5. The results showed that compared to MSLN CAR-T cells, CD40 scFv antibodies of different sequences had varying degrees of activation on DCs.

FIG. 7 provides the detection results of DC activation marker CD83 after co-incubation of different CAR-T cells under the conditions of DC: T=1:2.5 and 1:5. The results showed that compared to MSLN CAR-T cells, CD40 scFv antibodies of different sequences had varying degrees of activation on DCs.

FIG. 8 provides the detection results of DC activation marker CD86 after co-incubation of different CAR-T cells under the conditions of DC: T=1:2.5 and 1:5. The results showed that compared to MSLN CAR-T cells, CD40 scFv antibodies of different sequences had varying degrees of activation on DCs.

FIG. 9 provides the detection results of DC activation marker HLA-DR after co-incubation of different CAR-T cells under the conditions of DC: T=1:2.5 and 1:5. The results showed that compared to MSLN CAR-T cells, CD40 scFv antibodies of different sequences had varying degrees of activation on DCs.

FIG. 10 provides the detection results of the expression of CD137 activation marker after co-incubation of different CAR-T cells with U251, SKOV3-MSLN, SKOV3-MSLN/CD40, A549, NALM6, RPMI-8226 and THP1. The results showed that compared to MSLN CAR-T cells, the addition of CD40 scFv antibody molecules of different sequences did not change the specificity of the activation and killing effects of CAR-T cells on various tumor lines, that is, it did not affect the recognition specificity of MSLN CAR.

FIG. 11 provides the analysis of the therapeutic effects of different CAR-T cells on the SKOV3-MSLN/CD40 mouse tumor model. Different polylines represent different individual mice. The results show that compared to MSLN CAR-T cells without LACO molecules, A4044-MSLN CAR-T and A4052-MSLN CAR-T both have a better effect of enhancing tumor killing. A406-MSLN CAR-T, A409-MSLN CAR-T, A4025-MSLN CAR-T, and A4037-MSLN CAR-T have no or weak effect of enhancing tumor killing.

FIGS. 12A and 12B illustrate the vector structure for HER2 CAR and/or LACO expression. FIG. 12A shows the pUTCK-H13 schematic diagram of lentiviral vector structure. FIG. 12B shows the pUTCK-H13.LACO schematic diagram of lentiviral vector structure. LACO includes membrane LACO (mLACO) and soluble LACO (sLACO).

FIG. 13 illustrates anti-HER2 CAR-T and anti-HER2/LACO CAR-T cells, flow cytometry results stained with HER2-Fc antigen and anti-human IgG-Fc antibody. The staining results showed that the CAR molecules on anti-HER2 CAR-T and anti-HER2/LACO CAR-T cells were effectively expressed.

FIG. 14 illustrates the flow cytometry results of anti-HER2 CAR-T and anti-HER2/LACO CAR-T cells stained with CD40-Fc antigen and anti-human IgG-Fc antibody. The staining results showed that the expression level of LACO molecules on the CAR-T cells H13-A40517-CD28 expressing LACO on the cell membrane was very high. LACO molecules were not detected on the cell membrane of secreted LACO-expressing CAR-T cells H13-A4025-9.3h11, indicating that secreted LACO molecules may exist in soluble form outside the cells.

FIG. 15A-15C illustrate under the condition of E:T=3:1 (FIG. 15A), E:T=1:1 (FIG. 15B), and E:T=0.3:1 (FIG. 15C), the killing effect results of co-incubation of control NTD cells and 4 types of anti-HER2 CAR-T cells on SKOV3-CBG-GFP cells.

FIG. 16A-16C illustrate under the condition of E:T=3:1 (FIG. 16A), E:T=1:1 (FIG. 16B), and E:T=0.3:1 (FIG. 16C), the killing effect results of co-incubation of control NTD cells and 4 types of anti-HER2 CAR-T cells on A549-CBG-GFP cells. For A549 tumor cells with weak HER2 expression, the killing effect of H13, H13-A4025-9.3h11 and H13-A40517-CD28 was weaker than that of m4D5.

FIG. 17A-17F illustrate the structure of lentiviral vectors of CDH17-SR2 (FIG. 17A), CDH17-RSR (FIG. 17B), CDH17-R2S (FIG. 17C), CDH17-R2S.LACO (FIG. 17D), CDH17-SR2.LACO (FIG. 17E) and CDH17 (FIG. 17F).

FIG. 18A-18B illustrate flow cytometry results of anti-CDH17 CAR-T cells stained with 082 protein and anti-streptavidin antibody (FIG. 18A) or stained with PE anti-humanized VHH antibody (FIG. 18B).

FIG. 19A-19B illustrate flow cytometry results of anti-CDH17 CAR-T cells stained with 082 and Alexa Fluor™546 (REF A20183)-Rituximab antibody.

FIG. 20A-20C illustrate U87 cells electroporated with 10 μg CDH17 mRNA, U87 cells electroporated with 1 μg CDH17 mRNA and U87 cells were stained and tested the CDH17 expression levels by flow cytometry after 24 hours (FIG. 20A). The staining results showed that the CDH17 protein were effectively expressed on electroporated U87 cells. The CDH17 expression levels on ASPC1 cells (FIG. 20B) and A549 cells were also tested (FIG. 20C). In FIG. 20B and FIG. 20C, the left one cells were stained with CDH17 antibody and Alexa Fluor 647 goat anti-mouse IgG, and the right one cells were only stained with Alexa Fluor 647 goat anti-mouse IgG. CDH17 protein was expressed at high levels in ASPC1 and weakly in A549.

FIG. 21 illustrates under the condition of E:T=3:1, E:T=1:1, and E:T=0.3:1, the killing effect results of co-incubation with the control of NTD cells and VHH1 CAR-T cells on ASPC1 cells.

FIG. 22 illustrates under the condition of E:T=3:1, E:T=1:1, and E:T=0.3:1, the killing effect results of co-incubation with the control of NTD cells and VHH1 CAR-T cells on U87 cells, and U87 cells electroporated with 1 μg and 10 μg hCDH17 mRNA.

FIG. 23 illustrates under the condition of E:T=3:1, E:T=1:1, and E:T=0.3:1, the killing effect results of co-incubation with the control of NTD cells and VHH1 CAR-T cells on A549 cells.

FIG. 24-38 illustrate under the condition of E:T=3:1, the killing effect InCucyte analysis results of control NTD and VHH1 CAR-T cells responded to ASPC1 (FIG. 24), 293T (FIG. 25), 786-O (FIG. 26), A549 (FIG. 27), Caski (FIG. 28), H226 (FIG. 29), A375 (FIG. 30), HCC70 (FIG. 31), HepG2 (FIG. 32), Hu-7 (FIG. 33), PC3 (FIG. 34), SY5Y (FIG. 35), SKOV3 (FIG. 36), U251 (FIG. 37) and U87 (FIG. 38) tumor cells.

FIG. 39 illustrates the viability of CAR T cells with different treatments.

FIG. 40 illustrates flow cytometry results of anti-CDH17 CAR-T cells stained with 082 protein after CAR T-cells were incubated alone or in the presence of 100 μg/mL RTX and complement.

FIG. 41 illustrates the schematic structures of CD27-CAR and CD27-CAR-LACO.

FIG. 42A-42B shows the frequencies and medium fluorescence intensity (MFI) of CAR and LACO of the T cells of transduced with the designated CD27 CARs by using anti-CD27 and CD40-Fc flow staining.

FIG. 43 provides the cell expansion fold change of CAR-T cells.

FIG. 44A-44B provide results of the tumor killing assay showing the cytolytic activities of designated CAR-T cells against 786-O, U87, Skov-CD40, A549 and HepG2 at different E (T cells): T(tumor cells) ratio. FIG. 44A: E:T=1:1, 0.3:1; FIG. 44B: E:T=3:1, 1:1.

FIG. 45 provides ELISA results showing the production of IFN-γ and IL-2 by designated CART cells.

FIG. 46 provides the upregulated expression costimulatory molecules (CD80, CD86, CD83) and CD40 on the surface of DOHH2 cell line stimulated by designated CART cells.

FIG. 47 shows the schematic diagram of MSLN CAR/LACO.

FIG. 48 provide results of the tumor killing assay showing the cytolytic activities of MSLN CAR and different LACO CAR-T cells against A549 cells.

FIG. 49 provides the FACS staining results showing that the immature T cell derived from donor ND022 expressing anti-CD70 scFv can bind to CD70-Fc recombinant protein, and CAR-T cells co-expressing LACO containing anti-CD40 scFv can bind to CD40-Fc recombinant protein.

FIG. 50A-50C provides the killing curves of T cells derived from the healthy donor ND022 expressing LACO-anti-CD70 CAR by lentiviral transduction to different tumor cells at the E/T ratio of 1:3 or 1:1.

FIG. 51 provides the level of released cytokine IL-2 and IFN-gamma in cell supernatant after co-incubation of T cells derived from the healthy donor ND022 expressing LACO-anti-CD70 CAR by lentiviral transduction and tumor cells (293T, 786-O, U87) with different expression levels of endogenous CD70 of 24h.

FIG. 52 provides the FACS staining results showing that the immature T cell derived from donor ND020 expressing anti-CD70 scFv can bind to CD70-Fc recombinant protein, and CAR-T cells co-expressing LACO containing anti-CD40 scFv can bind to CD40-Fc recombinant protein.

FIG. 53 provides the level of released cytokine IL-2 and IFN-gamma in cell supernatant after co-incubation of T cells derived from the healthy donor ND022 expressing LACO-anti-CD70 CAR by lentiviral transduction and 786-O tumor cells.

FIG. 54 provides the killing curves of T cells derived from the healthy donor ND020 expressing LACO-anti-CD70 CAR by lentiviral transduction to different tumor cells at the E/T ratio of 3:1, 1:1 or 1:3.

FIG. 55 provides the effect on tumor volume after injection of different CAR-T cells.

### DETAILED DESCRIPTION

The present application provides novel antibodies, including antigen-binding fragments that specifically bind CD40 (e.g., human CD40). Pharmaceutical compositions comprising a therapeutically effective amount of such antibodies or antigen-binding fragments are also provided herein. Also provides herein are uses of such pharmaceutical compositions for treating cancer (e.g., CD40-expressing cancer) and methods of cancer treatment.

The present disclosure also provides a fusion protein comprising a first antigen-binding domain that specifically binds CD40, and a second antigen-binding domain that specifically binds CD28. The fusion proteins of the disclosure are also known as lymphocyte-antigen presenting cell co-stimulators (“LACO-Stims”). The expression of the fusion proteins disclosed herein not only promotes proliferation and activation of immune effector cells (e.g., T cells), but also stimulates maturation and epitope spreading activity of antigen presenting cells. In some embodiments, expression of the fusion proteins in genetically engineered T cells disclosed herein helps to overcome immunosuppression in tumor microenvironments mediated by, for example, PD1/PD-L1 signaling, regulatory T cells (Tregs), and TGF-β signaling, and enhance their anti-tumor activity.

### Definitions

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

CD40 is a 48 kD transmembrane glycoprotein surface receptor that is a member of the Tumor Necrosis Factor Receptor superfamily (TNFRSF). Exemplary amino acid sequences of human CD40 are described (see, e.g., Accession: ALQ33424.1, GenBank NP_001241.1, GI: 957949089), and SEQ ID NO: 141 is the amino acid sequence of human CD40. CD40 was initially characterized as a co-stimulatory receptor expressed on APCs that played a central role in B and T cell activation. The ligand for CD40, CD154 (also known as TRAP, T-BAM, CD40 Ligand or CD40L) is a type II integral membrane protein. See GenBank NP_001241.1 for reference to domains within CD40, for example, signal peptide, amino acids 1 to 20; extracellular domain, amino acids 21 to 193; transmembrane domain, amino acids 194 to 215; intracellular domain, amino acids 216 to 277.

CD28 is a protein expressed on T cells that provides co-stimulatory signals for T cell activation and survival. CD28 is the receptor for CD80 (B7.1) and CD86 (B7.2) proteins. A CD28 polypeptide can have an amino acid sequence corresponding to the sequence having GenBank No. P10747 (P10747.1, GI:115973) or NP_006130 (NP_006130.1, GI:5453611), the amino acid sequence of SEQ ID NO: 142, or fragments thereof. See GenBank NP_006130 for reference to domains within CD28, for example, signal peptide, amino acids 1 to 18; extracellular domain, amino acids 19 to 152; transmembrane domain, amino acids 153 to 179; intracellular domain, amino acids 180 to 220.

The term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgAQ1 and IgA2), based on the identity of their heavy-chain constant domains referred to as alpha, delta, epsilon, gamma, and mu, respectively. Unless expressly indicated otherwise, the term “antibody” as used herein include “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab, Fab′, F(ab′)2, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulin. In some instances, the Fv framework region residues of a human immunoglobulin are replaced with the corresponding residues in an antibody from a non-human species. In some instances, residues of the CDRs are replaced by residues from the CDRs of a non-human species (e.g., mouse, rat, hamster, camel) that have the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The term “human antibody” as used herein refers to an antibody produced by a human or an antibody having an amino acid sequence corresponding to an antibody produced by a human made using any of the techniques known in the art.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (a), delta (δ), epsilon (ε), gamma (γ) and mu (μ), based on the amino acid sequence of the heavy chain constant region. The distinct heavy chains differ in size: α, δ and γ contain approximately 450 amino acids, while and F contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5thed. A variable region can be a human variable region.

A CDR refers to one of three hypervariable regions (H1, H2 or H3) within the non-framework region of the immunoglobulin (Ig or antibody) VH 3-sheet framework, or one of three hypervariable regions (L1, L2 or L3) within the non-framework region of the antibody VL 3-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat et al, J. Biol. Chem. 252:6609-6616 (1977); Kabat, Adv. Prot. Chem. 32: 1-75 (1978)). The Chothia definition is based on the location of the structural loop regions, which defines CDR region sequences as those residues that are not part of the conserved p-sheet framework, and thus are able to adapt different conformations (Chothia and Lesk, J. Mol. Biol. 196:901-917 (1987)). Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures (Al-Lazikani et al, J. Mol. Biol. 273:927-948 (1997); Morea et al, Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth in the table below.

| | Kabat¹ | Chothia² | Loop Location |
|---|---|---|---|
| VH CDRl | 31-35 | 26-32 | linking B and C strands |
| HCDR2 | 50-65 | 53-55 | linking C′ and C″ strands |
| HCDR3 | 95-102 | 96-101 | linking F and G strands |
| VL CDRl | 24-34 | 26-32 | linking B and C strands |
| LCDR2 | 50-56 | 50-52 | linking C′ and C″ strands |
| LCDR3 | 89-97 | 91-96 | linking F and G strands |

¹Residue numbering follows the nomenclature of Kabat et al., supra
²Residue numbering follows the nomenclature of Chothia et al., supra

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to bind to a particular antigen of interest. The CDR regions can be analyzed by, for example, abysis website (http://abysis.org/).

Thus, unless otherwise specified, a CDR, or individual specified CDRs (e.g., LCDR1, LCDR2, LCDR3), of a given antibody or region thereof, such as a variable region thereof, should be understood to encompass a (or the specific) complementary determining region as defined by any of the aforementioned schemes, or other known schemes. For example, where it is stated that a particular CDR (e.g., a HCDR3) contains the amino acid sequence of a corresponding CDR in a given VH or VL region amino acid sequence, it is understood that such a CDR has a sequence of the corresponding CDR (e.g., HCDR3) within the variable region, as defined by any of the aforementioned schemes, or other known schemes. In some embodiments, specific CDR sequences are specified, although it is understood that a provided antibody can include CDRs as described according to any of the other aforementioned numbering schemes or other numbering schemes known to a skilled artisan. Likewise, unless otherwise specified, a FR or individual specified FR(s) (e.g., VH FR1, VH FR2, VH FR3, VH FR4), of a given antibody or region thereof, such as a variable region thereof, should be understood to encompass a (or the specific) framework region as defined by any of the known schemes.

The terms “epitope” and “antigenic determinant” are used interchangeably herein an refer to the site on the surface of a target molecule to which an antibody or antigen-binding fragment binds, such as a localized region on the surface of an antigen. The target molecule can comprise, a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three-dimensional structural characteristics as well as specific charge characteristics. The term, “epitope” includes linear epitopes and conformational epitopes. A region of a target molecule (e.g., a polypeptide) contributing to an epitope can be contiguous amino acids of the polypeptide or the epitope can come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified, for example, by immunoassays, ELISAs, SPR (e.g., Biacore), or other techniques known to those of skill in the art. Typically, a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a KD of about 0.1 mM or less. In some embodiments, “specifically binds” means that a polypeptide or molecule binds a target with a KD of at about 10 μM or less or about 1 μM or less. In some embodiments, “specifically binds” means that a polypeptide or molecule binds a target with a KD of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “binding affinity” as used herein generally refers to the strength of the sum total of noncovalent interactions between a binding moiety and a target molecule (e.g., antigen). The binding of a binding moiety and a target molecule is a reversible process, and the affinity of the binding is typically reported as an equilibrium dissociation constant (KD). KD is the ratio of a dissociation rate (koff or kd) to the association rate (kon or ka). The lower the KD of a binding pair, the higher the affinity. A variety of methods of measuring binding affinity are known in the art, any of which can be used for purposes of the present disclosure. Specific illustrative embodiments include the following. In some embodiments, the “KD” or “KD value” can be measured by assays known in the art, for example by a binding assay. The KD may be measured in a radiolabeled antigen binding assay (RIA) (Chen, et al., (1999) J. Mol Biol 293:865-881). The KD or KD value may also be measured by using surface plasmon resonance assays by Biacore, using, for example, a BIAcore™-2000 or a BIAcore™-3000 BIAcore, Inc., Piscataway, NJ), or by biolayer interferometry using, for example, the OctetQK384 system (ForteBio, Menlo Park, CA).

The term “variant” as used herein in relation to a protein or a polypeptide with particular sequence features (the “reference protein” or “reference polypeptide”) refers to a different protein or polypeptide having one or more (such as, for example, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. A functional fragment or a functional variant of a protein or polypeptide maintains the basic structural and functional properties of the reference protein or polypeptide.

The terms “polypeptide,” “peptide,” “protein,” and their grammatical equivalents as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The term “fusion protein” as used herein refers to a protein, peptide or polypeptide whose amino acid sequence is derived from two or more isolated proteins, peptides or polypeptides. The fusion proteins also include amino acid linking regions between amino acid portions from the isolated proteins, peptides or polypeptides. Such a linker region of amino acids is herein referred to as a “linker”.

The terms “polynucleotide,” “nucleic acid,” and their grammatical equivalents as used interchangeably herein mean polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., an anti-CD40 antibody or antigen-binding fragment as described herein), and it is further understood that expression levels can be optimized to obtain sufficient expression using methods well known in the art.

The term “chimeric antigen receptor” or “CAR” as used herein refers to an artificially constructed hybrid protein or polypeptide containing a binding moiety (e.g., an antibody) linked to immune cell (e.g., T cell) signaling or activation domains. In some embodiments, CARs are synthetic receptors that retarget T cells to tumor surface antigens (Sadelain et al., Nat. Rev. Cancer 3(1):35-45 (2003); Sadelain et al., Cancer Discovery 3(4):388-398 (2013)). CARs can provide both antigen binding and immune cell activation functions onto an immune cell such as a T cell. CARs have the ability to redirect T-cell specificity and reactivity toward a selected target in a non-MHC-restricted manner, exploiting the antigen-binding properties of monoclonal antibodies. The non-MHC-restricted antigen recognition can give T-cells expressing CARs the ability to recognize an antigen independent of antigen processing, thus bypassing a mechanism of tumor escape.

The term “genetic engineering” or its grammatical equivalents when used in reference to a cell is intended to mean alteration of the genetic materials of the cell that is not normally found in a naturally occurring cell. Genetic alterations include, for example, modifications introducing expressible polynucleotides, other additions, mutations/alterations, deletions and/or other functional disruption of the cell's genes. Such modifications can be done in, for example, coding regions and functional fragments thereof of a gene. Additional modifications can be done in, for example, non-coding regulatory regions in which the modifications alter expression of a gene.

The term “transfer,” “transduce,” “transfect,” and their grammatical equivalents as used herein refer to a process by which an exogenous polynucleotide is introduced into the host cell. A “transferred,” “transfected,” or “transduced” cell is one which has been transferred, transduced, or transfected with an exogenous polynucleotide. The cell includes the primary subject cell and its progeny. A polynucleotide can be “transferred” into a host cell using any type of approaches known in the art, including, e.g., a chemical method, a physical method, or a biological method. A polynucleotide is commonly “transduced” into a host cell using a virus. By contrast, a polynucleotide is commonly “transfected” into a host cell using a non-viral approach. These terms are used interchangeable at times, and a person of ordinary skill in the art would readily understand their meanings in different contexts.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure.

The term “immune effector cell” and its grammatical equivalents as used herein and understood in the art refer to cells that are of hematopoietic origin and play a direct role in the immune response against a target, such as a pathogen, a cancer cell, or a foreign substance. Immune effector cells include T cells, B cell, natural killer (NK) cells, NKT cells, macrophages, granulocytes, neutrophils, eosinophils, mast cells, and basophils.

“Stimulation” of an immune effector cell means a primary response induced by binding of a stimulatory molecule with its cognate ligand thereby mediating a signal transduction event in the immune effector cell which can alter expression of certain genes and/or reorganization of cytoskeletal structures, and the like. A “stimulatory molecule” of an immune effector cell refers to a molecule on the immune effector cell that, upon binding with its cognate ligand, which is commonly present on an APC, can mediate signal transduction to promote the maturation, differentiation, proliferation, and/or activation of the immune effector cell. For example, a stimulatory molecule of the T cells, the TCR/CD3 complex triggers the activation of the T cells. The ligand for a stimulatory molecule, or “stimulatory ligand,” means a ligand that is commonly present on an APC and can bind with a stimulatory molecule on the immune effector cell to mediate a primary response by the immune effector cell, including, but not limited to, maturation, differentiation, activation, initiation of an immune response, proliferation, and the like. Stimulatory ligands are well-known in the art and encompass, for example, an MHC Class I molecule loaded with a peptide, an anti-CD3 antibody, a superagonist anti-CD28 antibody, and a superagonist anti-CD2 antibody.

A “co-stimulatory signal,” as used herein and understood in the art, refers to a signal from a co-stimulatory receptor (e.g., CD28 or 4-1n), which in combination with a primary signal (e.g., TCR/CD3) promotes optimal clonal expansion, differentiation and effector functions of immune effector cells (e.g., T cells). A “co-stimulatory receptor” of an immune effector cell, s used herein and understood in the art, refers to a molecule on the immune effector cell that specifically binds with a “co-stimulatory ligand” to mediate a co-stimulatory response by the immune effector cell, such as heightened activation or proliferation of the immune effector cell. Co-stimulatory receptors for immune effector cells include, but are not limited to, CD28, 4-1BB, ICOS, CD27, OX40, DAP10, CD30, 2B4, CD2, LIGHT, GITR, TLR, DR3, and CD43. A “functional fragment” of a co-stimulatory receptor is a fragment of the co-stimulatory receptor that retains its function to mediate a co-stimulatory signal and stimulate the immune effector cell. In some embodiments, a functional fragment of a co-stimulatory receptor retains the co-stimulatory domain of the co-stimulatory receptor. In some embodiments, the co-stimulatory domain is the cytoplasmic domain of the co-stimulatory receptor. In some embodiments, signals from co-stimulatory receptors of immune effector cells (e.g., T cells) lower the activation threshold for the immune effector cells. In some embodiments, signals from co-stimulatory receptors of T cells lead to the augmentation of TCR signaling events necessary for efficient cytokine production (via augmented transcriptional activity and messenger RNA stabilization), cell cycle progression, survival, regulation of metabolism and T cell responses.

A “co-stimulatory ligand,” as used herein and understood in the art, refers to a molecule that specifically binds a cognate co-stimulatory receptor on an immune effector cell, thereby providing a signal which, in addition to the primary signal provided by the stimulatory molecule, mediates a response in the immune effector cell, including, but not limited to, proliferation, activation, differentiation, and the like. The co-stimulatory ligand can be present on an APC (e.g., a dendritic cell). Co-stimulatory ligands include, but are not limited to, CD58, CD70, CD83, CD80, CD86, CD137L (4-1BBL), CD252 (OX40L), CD275 (ICOS-L), CD54 (ICAM-1), CD49a, CD112 (PVRL2), CD150 (SLAM), CD155 (PVR), CD265 (RANK), CD270 (HVEM), TL1A, CD127, IL-4R, GITR-L, TIM-4, CD153 (CD30L), CD48, CD160, CD200R (OX2R), and CD44. A “receptor-binding fragment” of a co-stimulatory ligand refers to a fragment of the ligand that retains its capacity to bind its receptor.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated. For example, when used in reference to a cancer or tumor, the term “treat” and its grammatical equivalents refer to an action that reduces the severity of the cancer or tumor, or retards or slows the progression of the cancer or tumor, including (a) inhibiting the growth, or arresting development of the cancer or tumor, (b) causing regression of the cancer or tumor, or (c) delaying, ameliorating or minimizing one or more symptoms associated with the presence of the cancer or tumor.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition.

The term “subject” as used herein refers to any animal (e.g., a mammal), including, but not limited to, humans, non-human primates, canines, felines, rodents, and the like, which is to be the recipient of a particular treatment. A subject can be a human. A subject can have a particular disease or condition.

The term “autologous” as used herein refers to any material derived from the same individual to which it is later to be re-introduced into the individual.

The term “allogeneic” as used herein refers to a graft derived from a different animal of the same species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOs. It is understood that one skilled in the art can readily identify homologous sequences by reference to sequence sources, including but not limited to GenBank (ncbi.nlm.nih.gov/genbank/) and EMBL (embl.org/).

### Anti-CD40 Antibodies and Antigen-Binding Fragments

Provided herein are antibodies or antigen-binding fragments thereof that specifically bind CD40 (e.g., human CD40). In some embodiments, provided herein are anti-CD40 antibodies. In some embodiments, the antibody is an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, provided herein are antigen-binding fragments of an anti-CD40 antibody. In some embodiments, antigen-binding fragments provided herein can be a single domain antibody (sdAb), a heavy chain antibody (HCAb), a Fab, a Fab′, a F(ab′)2, a Fv, a single-chain variable fragment (scFv), or a (scFv)2. In some embodiments, the antigen-binding fragment of an anti-CD40 antibody is a single domain antibody (sdAb).

In some embodiments, the anti-CD40 antibodies or antigen-binding fragments provided herein comprise recombinant antibodies or antigen-binding fragments. In some embodiments, the anti-CD40 antibodies or antigen-binding fragments provided herein comprise monoclonal antibodies or antigen-binding fragments. In some embodiments, the anti-CD40 antibodies or antigen-binding fragments provided herein comprise polyclonal antibodies or antigen-binding fragments. In some embodiments, the anti-CD40 antibodies or antigen-binding fragments provided herein comprise camelid (e.g., camels, dromedary and llamas) antibodies or antigen-binding fragments. In some embodiments, the anti-CD40 antibodies or antigen-binding fragments provided herein comprise chimeric antibodies or antigen-binding fragments. In some embodiments, the anti-CD40 antibodies or antigen-binding fragments provided herein comprise humanized antibodies or antigen-binding fragments. In some embodiments, the anti-CD40 antibodies or antigen-binding fragments provided herein comprise human antibodies or antigen-binding fragments. In some embodiments, provided herein are anti-CD40 human scFvs.

In some embodiments, the anti-CD40 antibodies or antigen-binding fragments provided herein are isolated. In some embodiments, the anti-CD40 antibodies or antigen-binding fragments provided herein are substantially pure.

In some embodiments, the anti-CD40 antibody or antigen-binding fragment provided herein comprises a multispecific antibody or antigen-binding fragment. In some embodiments, the anti-CD40 antibody or antigen-binding fragment provided herein comprises a bispecific antibody or antigen-binding fragment. In some embodiments, provided herein is a Bi-specific T-cell engager (BiTE). BiTEs are bispecific antibodies that bind to a T cell antigen (e.g., CD3) and a tumor antigen. BiTEs have been shown to induce directed lysis of target tumor cells and thus provide great potential therapies for cancers and other disorders. In some embodiments, provided herein are BiTEs that specifically bind CD3 and CD40. In some embodiments, the BiTEs comprises an anti-CD40 antibody or antigen-binding fragment provided herein. In some embodiments, the BiTEs comprises an anti-CD40 scFv provided herein.

In some embodiments, the anti-CD40 antibody or antigen-binding fragment provided herein comprises a monovalent antigen-binding site. In some embodiments, an anti-CD40 antibody or antigen-binding fragment comprises a monospecific binding site. In some embodiments, an anti-CD40 antibody or antigen-binding fragment comprises a bivalent binding site.

In some embodiments, an anti-CD40 antibody or antigen-binding fragment is a monoclonal antibody or antigen-binding fragment. Monoclonal antibodies can be prepared by any method known to those of skill in the art. One exemplary approach is screening protein expression libraries, e.g., phage or ribosome display libraries. Phage display is described, for example, in Ladner et al., U.S. Pat. No. 5,223,409; Smith (1985) Science 228:1315-1317; and WO 92/18619. In some embodiments, recombinant monoclonal antibodies are isolated from phage display libraries expressing variable regions or CDRs of a desired species. Screening of phage libraries can be accomplished by various techniques known in the art.

In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a human protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, BLI, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, an anti-CD40 antibody or antigen-binding fragment is a humanized antibody or antigen-binding fragment. Various methods for generating humanized antibodies are known in the art. Methods are known in the art for achieving high affinity binding with humanized antibodies. A non-limiting example of such a method is hypermutation of the variable region and selection of the cells expressing such high affinity antibodies (affinity maturation). In addition to the use of display libraries, the specified antigen (e.g., recombinant CD40 or an epitope thereof) can be used to immunize a non-human animal, e.g., a rodent. In certain embodiments, rodent antigen-binding fragments (e.g., mouse antigen-binding fragments) can be generated and isolated using methods known in the art and/or disclosed herein. In some embodiments, a mouse can be immunized with an antigen (e.g., recombinant CD40 or an epitope thereof).

In some embodiments, an anti-CD40 antibody or antigen-binding fragment is a human antibody or antigen-binding fragment. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, can be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

The specific CDR sequences defined herein are generally based on a combination of Kabat and Chothia definitions. However, it is understood that reference to a heavy chain CDR or CDRs and/or a light chain CDR or CDRs of a specific antibody encompass all CDR definitions as known to those of skill in the art.

Anti-CD40 antibodies or antigen-binding fragments provided herein include the followings scFv clones: 406, 409, 4011, 4012, 4021, 4025, 4034, 4037, 4044, 4045 and 4052. The sequence features are described below.

In some embodiments, anti-CD40 antibodies or antigen-binding fragments provided herein comprise one, two, three, four, five, and/or six CDRs of any one of the antibodies described herein. In some embodiments, anti-CD40 antibodies or antigen-binding fragments provided herein comprise one, two, three, four, five, and/or six CDRs of 406, 409, 4011, 4012, 4021, 4025, 4034, 4037, 4044, 4045 and 4052. In some embodiments, anti-CD40 antibodies or antigen-binding fragments provided herein comprise a VL comprising one, two, and/or three, LCDRs from Table 1. In some embodiments, anti-CD40 antibodies or antigen-binding fragments provided herein comprise a VH comprising one, two, and/or three HCDRs from Table 2. In some embodiments, anti-CD40 antibodies or antigen-binding fragments provided herein comprise one, two, and/or three LCDRs from Table 1 and one, two, and/or three HCDRs from Table 2.

TABLE 1
Amino acid sequences of light chain variable
region CDRs (LCDRs) of anti-CD40 Abs

| Clone | LCDR1 | LCDR2 | LCDR3 |
|---|---|---|---|
| 406 | SEQ ID NO: 6 | SEQ ID NO: 7 | SEQ ID NO: 8 |
| 409 | SEQ ID NO: 15 | SEQ ID NO: 16 | SEQ ID NO: 17 |
| 4011 | SEQ ID NO: 22 | SEQ ID NO: 23 | SEQ ID NO: 24 |
| 4012 | SEQ ID NO: 31 | SEQ ID NO: 32 | SEQ ID NO: 33 |
| 4021 | SEQ ID NO: 40 | SEQ ID NO: 41 | SEQ ID NO: 42 |
| 4025 | SEQ ID NO: 49 | SEQ ID NO: 50 | SEQ ID NO: 51 |
| 4034 | SEQ ID NO: 58 | SEQ ID NO: 41 | SEQ ID NO: 59 |
| 4037 | SEQ ID NO: 66 | SEQ ID NO: 67 | SEQ ID NO: 68 |
| 4044 | SEQ ID NO: 71 | SEQ ID NO: 72 | SEQ ID NO: 51 |
| 4045 | SEQ ID NO: 79 | SEQ ID NO: 80 | SEQ ID NO: 81 |
| 4052 | SEQ ID NO: 88 | SEQ ID NO: 89 | SEQ ID NO: 90 |

TABLE 2
Amino acid sequences of heavy chain variable
region CDRs (HCDRs) of anti-CD40 Abs

| Clone | HCDR1 | HCDR2 | HCDR3 |
|---|---|---|---|
| 406 | SEQ ID NO: 2 | SEQ ID NO: 3 | SEQ ID NO: 4 |
| 409 | SEQ ID NO: 11 | SEQ ID NO: 12 | SEQ ID NO: 13 |
| 4011 | SEQ ID NO: 2 | SEQ ID NO: 3 | SEQ ID NO: 20 |
| 4012 | SEQ ID NO: 27 | SEQ ID NO: 28 | SEQ ID NO: 29 |
| 4021 | SEQ ID NO: 36 | SEQ ID NO: 37 | SEQ ID NO: 38 |
| 4025 | SEQ ID NO: 45 | SEQ ID NO: 46 | SEQ ID NO: 47 |
| 4034 | SEQ ID NO: 54 | SEQ ID NO: 55 | SEQ ID NO: 56 |
| 4037 | SEQ ID NO: 62 | SEQ ID NO: 63 | SEQ ID NO: 64 |
| 4044 | SEQ ID NO: 45 | SEQ ID NO: 46 | SEQ ID NO: 47 |
| 4045 | SEQ ID NO: 75 | SEQ ID NO: 76 | SEQ ID NO: 77 |
| 4052 | SEQ ID NO: 84 | SEQ ID NO: 85 | SEQ ID NO: 86 |

In some embodiments, an anti-CD40 antibody or antigen-binding fragment thereof comprises a humanized antibody or antigen-binding fragment. In some embodiments, an anti-CD40 antibody or antigen-binding fragment thereof comprises a LCDR1, LCDR2, LCDR3, HCDR1, HCDR2, and/or HCDR3 from an antibody or antigen-binding fragment described herein. In some embodiments, an anti-CD40 antibody or antigen-binding fragment thereof comprises a variant of an anti-CD40 antibody or antigen-binding fragment described herein. In some embodiments, a variant of an anti-CD40 antibody or antigen-binding fragment comprises one to 30 amino acid substitutions, additions, and/or deletions in the anti-CD40 antibody or antigen-binding fragment. In some embodiments, a variant of an anti-CD40 antibody or antigen-binding fragment comprises one to 25 amino acid substitutions, additions, and/or deletions in the anti-CD40 antibody or antigen-binding fragment. In some embodiments, a variant of an anti-CD40 antibody or antigen-binding fragment comprises one to 20 substitutions, additions, and/or deletions in the anti-CD40 antibody or antigen-binding fragment. In some embodiments, a variant of an anti-CD40 antibody or antigen-binding fragment comprises one to 15 substitutions, additions, and/or deletions in the anti-CD40 antibody or antigen-binding fragment. In some embodiments, a variant of an anti-CD40 antibody or antigen-binding fragment comprises one to 10 substitutions, additions, and/or deletions in the anti-CD40 antibody or antigen-binding fragment. In some embodiments, a variant of an anti-CD40 antibody or antigen-binding fragment comprises one to five conservative amino acid substitutions, additions, and/or deletions in the anti-CD40 antibody or antigen-binding fragment. In some embodiments, a variant of an anti-CD40 antibody or antigen-binding fragment comprises one to three amino acid substitutions, additions, and/or deletions in the anti-CD40 antibody or antigen-binding fragment. In some embodiments, the amino acid substitutions, additions, and/or deletions are conservative amino acid substitutions. In some embodiments, the conservative amino acid substitution(s) is in a CDR of the antibody or antigen-binding fragment. In some embodiments, the conservative amino acid substitution(s) is not in a CDR of the antibody or antigen-binding fragment. In some embodiments, the conservative amino acid substitution(s) is in a framework region of the antibody or antigen-binding fragment.

In some embodiments, provided herein are antibodies or antigen-binding fragments thereof that specifically bind CD40 having a VL and a VH. In some embodiments, the VL and VH are connected by a linker. The linker can be a flexible linker or a rigid linker. In some embodiments, the linker has the amino acid sequence of (GGGGS)n, n=3,4, or 5 (SEQ ID NO: 97). In some embodiments, the linker has the amino acid sequence of (EAAAK)n, n=3, 4, or 5 (SEQ ID NO: 98)). In some embodiments, the linker has the amino acid sequence of (PA)nP, n=1, 2, 3, 4, or 5 (SEQ ID NO: 99). In some embodiments, the linker has the amino acid sequence of GGGGSGGGGSGGGGS (SEQ ID NO: 100).

In some embodiments, anti-CD40 antibodies or antigen-binding fragments provided herein comprise the VL and/or the VH of any one of the antibodies described herein. In some embodiments, anti-CD40 antibodies or antigen-binding fragments provided herein comprise the VL and/or the VH of the scFv designated as 406, 409, 4011, 4012, 4021, 4025, 4034, 4037, 4044, 4045 and 4052.

TABLE 3
Amino acid sequences of light chain variable regions (VLs) and
heavy chain variable region (VHs) of anti-CD40 antibodies

| Clone | VH | VL |
|---|---|---|
| 406 | SEQ ID NO: 1 | SEQ ID NO: 5 |
| 409 | SEQ ID NO: 10 | SEQ ID NO: 14 |
| 4011 | SEQ ID NO: 19 | SEQ ID NO: 21 |
| 4012 | SEQ ID NO: 26 | SEQ ID NO: 30 |
| 4021 | SEQ ID NO: 35 | SEQ ID NO: 39 |
| 4025 | SEQ ID NO: 44 | SEQ ID NO: 48 |
| 4034 | SEQ ID NO: 53 | SEQ ID NO: 57 |
| 4037 | SEQ ID NO: 61 | SEQ ID NO: 65 |
| 4044 | SEQ ID NO: 44 | SEQ ID NO: 70 |
| 4045 | SEQ ID NO: 74 | SEQ ID NO: 78 |
| 4052 | SEQ ID NO: 83 | SEQ ID NO: 87 |

In some embodiments, provided herein are antibodies or antigen-binding fragments thereof that specifically bind CD40 comprising a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 5, 14, 21, 30, 39, 48, 57, 65, 70, 78 and 87. In some embodiments, provided herein are antibodies or antigen-binding fragments thereof that specifically bind CD40 comprising a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 1, 10, 19, 26, 35, 44, 53, 61, 74 and 83.

In some embodiments, provided herein are antibodies or antigen-binding fragments thereof that specifically bind CD40 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to an amino acid sequence selected from the group consisting of SEQ ID NOs: 5, 14, 21, 30, 39, 48, 57, 65, 70, 78 and 87; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to an amino acid sequence selected from the group consisting of SEQ ID NOs: 1, 10, 19, 26, 35, 44, 53, 61, 74 and 83.

TABLE 4
Amino acid sequences of scFv of anti-CD40 antibodies

| Clone | scFV |
|---|---|
| 406 | SEQ ID NO: 9 |
| 409 | SEQ ID NO: 18 |
| 4011 | SEQ ID NO: 25 |
| 4012 | SEQ ID NO: 34 |
| 4021 | SEQ ID NO: 43 |
| 4025 | SEQ ID NO: 52 |
| 4034 | SEQ ID NO: 60 |
| 4037 | SEQ ID NO: 69 |
| 4044 | SEQ ID NO: 73 |
| 4045 | SEQ ID NO: 82 |
| 4052 | SEQ ID NO: 91 |

In some embodiments, provided herein are antibodies or antigen-binding fragments thereof that specifically bind CD40 comprising a scFv having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to an amino acid sequence selected from the group consisting of SEQ ID NOs: 9, 18, 25, 34, 43, 52, 60, 69, 73, 82 and 91.

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations can be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions can be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant can be determined by systematically making insertions, deletions, or substitutions in the sequence and testing the resulting variant proteins for activity as compared to the parent protein.

In some embodiments, anti-CD40 antibodies or antigen-binding fragments described herein bind to human CD40 with high affinity, for example, with a KD of 10⁻⁷ M or less, 10⁻⁸ M or less, 5×10⁻⁹ M or less, 10⁻⁹ M or less, 5×10⁻¹⁰ M or less, 10⁻¹¹ M or less, 5×10⁻¹¹ M or less, 10⁻¹¹ M or less, 5×10⁻¹² M or less, 10⁻¹² M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, 10⁻⁸ M to 10⁻⁷ M, 10⁻¹⁰ M to 10⁻⁸ M, 10⁻⁹ M to 10⁻⁸ M, 10⁻¹¹ M to 10⁻⁹ M, or 10⁻¹⁰ M to 10⁻⁹ M. In some embodiments, anti-CD40 antibodies or antigen-binding fragments described herein bind to human CD40 with a KD of 10⁻¹¹ M to 5×10⁻⁹ M. In some embodiments, anti-CD40 antibodies or antigen-binding fragments described herein bind to soluble human CD40 with high affinity, e.g., as determined by BLI, with a KD of 10⁻⁷ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, 10⁻¹¹ M or less, 5×10⁻¹² M or less, 10⁻¹² M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, 10⁻⁸ M to 10⁻⁷, 10⁻¹⁰ M to 10⁻⁸ M, 10⁻⁹ M to 10⁻⁸ M, 10⁻¹¹ M to 10⁻⁹ M, or 10⁻¹⁰ M to 10⁻⁹ M. In some embodiments, anti-CD40 antibodies or antigen-binding fragments described herein bind to soluble human CD40 with a KD of 10⁻¹¹ M to 5×10⁻⁹ M. In some embodiments, anti-CD40 antibodies or antigen-binding fragments described herein bind to bound (e.g., cell membrane bound) human CD40, such as on activated human T cells, e.g., as determined by flow cytometry and Scatchard plot, with a KD of 10⁻⁷ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, 10⁻¹¹ M or less, 5×10⁻¹² M or less, 10⁻¹² M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, 10⁻⁸ M to 10⁻⁷ M, 10⁻¹⁰ M to 10⁻⁸ M, 10⁻⁹ M to 10⁻⁸ M, 10⁻¹¹ M to 10⁻⁹ M, or 10⁻¹⁰ M to 10⁻⁹ M. In some embodiments, an anti-CD40 antibody or antigen-binding fragment binds to bound (e.g., cell membrane bound) human CD40, such as on activated human T cells, e.g., as determined by flow cytometry, with an EC50 of 10 μg/mL or less, 5 μg/mL or less, 1 μg/mL or less, 0.9 μg/mL or less, 0.8 μg/mL or less, 0.7 μg/mL or less, 0.6 μg/mL or less, 0.5 μg/mL or less, 0.4 μg/mL or less, 0.3 μg/mL or less, 0.2 μg/mL or less, 0.1 μg/mL or less, 0.05 μg/mL or less, or 0.01 μg/mL or less.

### LACO-Stim Fusion Proteins

Provided herein are also fusion proteins comprising a first domain that activates an antigen-presenting cell (“APC”; e.g., a dendritic cell) and a second domain that activates an immune effector cell (e.g., a T cell), wherein the first domain comprises an anti-CD40 antibody or an antigen-binding fragment thereof disclosed herein, and the second domain comprises (a) a co-stimulatory receptor of the immune effector cell, or a functional fragment thereof, (b) a co-stimulatory ligand of the immune effector cell, or a receptor-binding fragment thereof, or (c) an antibody that binds a co-stimulatory receptor of the immune effector cell, or an antigen-binding fragment thereof. Such fusion proteins are also referred to as Lymphocytes-APCs Co-stimulators (“LACO-Stim” molecules or “LACO” molecules).

In some embodiments, the fusion protein is a membrane protein. In some embodiments, the fusion protein is a soluble protein. In some embodiments, the fusion protein is a bispecific antibody. In some embodiments, the C-terminus of the first domain is linked to the N-terminus of the second domain. In some embodiments, the N-terminus of the first domain is linked to the C-terminus of the second domain. In some embodiments, the first domain and the second domain are linked via a linker. The linker can be a flexible linker or a rigid linker. In some embodiments, the linker has the amino acid sequence of (GGGGS)n, n=1, 2, 3, 4, or 5 (SEQ ID NO: 97). In some embodiments, the linker has the amino acid sequence of (EAAAK)n, n=1, 2, 3, 4, or 5 (SEQ ID NO: 98). In some embodiments, the linker has the amino acid sequence of (PA)nP, n=1, 2, 3, 4, or 5 (SEQ ID NO: 99, the amino sequence of SEQ ID NO: 99 is (PA)nP, n=1,2,3,4, or 5). In some embodiments, the linker has the amino acid sequence of GSGGGGSGGGGSGGGGS (SEQ ID NO: 144). In some embodiments, the linker is a CD8 hinge (SEQ ID NO: 126). In some embodiments, the linker is a CD28 hinge (SEQ ID NO: 145). In some embodiments, the linker is an IgG Fc hinge (SEQ ID NO: 146). In some embodiments, the linker can be a trimerization motif selected from the group consisting of a T4 fibritin trimerization motif (SEQ ID NO: 147), an isoleucine zipper (SEQ ID NO: 148 or 149), a GCN4II motif (SEQ ID NO:150 or 151), a Matrilin-1 motif (SEQ ID NO: 152 or 153), and a collagen XV trimerization motif (SEQ ID NO: 154).

In some embodiments, the fusion protein provided herein provided herein comprises a multi-specific antibody or antigen-binding fragment. In some embodiments, the fusion protein provided herein comprises a bispecific antibody or antigen-binding fragment. In some embodiments, the fusion protein provided herein may further comprise one or more Fc domain.

In some embodiments, the fusion protein further comprises a signal peptide domain. In some embodiments, the C-terminus of the signal peptide is linked to the N-terminus of the first domain. In some embodiments, the signal peptide comprises a signal peptide derived from CD8. In some embodiments, fusion proteins provided herein further comprise a transmembrane region.

“Immune effector cells” as used herein and understood in the art refer to cells that are of hematopoietic origin and play a direct role in the immune response against a target, such as a pathogen, a cancer cell, or a foreign substance. Immune effector cells include T cells, B cell, natural killer (NK) cells, NKT cells, macrophages, granulocytes, neutrophils, eosinophils, mast cells, and basophils. In some embodiments, the second domain of the fusion proteins provided herein that activates an immune effector cell comprises a co-stimulatory receptor of the immune effector cell. In some embodiments, the immune effector cell is a T cell, an NK cell, an NKT cell, a macrophage, a neutrophil, or a granulocyte. In some embodiments, the immune effector cell is a T cell. In some embodiments, the immune effector cell is a NK cell. In some embodiments, the immune effector cell is a macrophage.

“Stimulation” of an immune effector cell means a primary response induced by binding of a stimulatory molecule with its cognate ligand thereby mediating a signal transduction event in the immune effector cell which can alter expression of certain genes and/or reorganization of cytoskeletal structures, and the like. A “stimulatory molecule” of an immune effector cell refers to a molecule on the immune effector cell that, upon binding with its cognate ligand, which is commonly present on an APC, can mediate signal transduction to promote the maturation, differentiation, proliferation, and/or activation of the immune effector cell. For example, a stimulatory molecule of the T cells, the TCR/CD3 complex triggers the activation of the T cells. The ligand for a stimulatory molecule, or “stimulatory ligand,” means a ligand that is commonly present on an APC and can bind with a stimulatory molecule on the immune effector cell to mediate a primary response by the immune effector cell, including, but not limited to, maturation, differentiation, activation, initiation of an immune response, proliferation, and the like. Stimulatory ligands are well-known in the art and encompass, for example, an MHC Class I molecule loaded with a peptide, an anti-CD3 antibody, a superagonist anti-CD28 antibody, and a superagonist anti-CD2 antibody.

A “co-stimulatory signal,” as used herein and understood in the art, refers to a signal from a co-stimulatory receptor (e.g., CD28 or 4-1BB), which in combination with a primary signal (e.g., TCR/CD3) promotes optimal clonal expansion, differentiation and effector functions of immune effector cells (e.g., T cells). A “co-stimulatory receptor” of an immune effector cell, s used herein and understood in the art, refers to a molecule on the immune effector cell that specifically binds with a “co-stimulatory ligand” to mediate a co-stimulatory response by the immune effector cell, such as heightened activation or proliferation of the immune effector cell. Co-stimulatory receptors for immune effector cells include, but are not limited to, CD28, 4-1BB, ICOS, CD27, OX40, DAP10, CD30, 2B4, CD2, LIGHT, GITR, TLR, DR3, and CD43. A “functional fragment” of a co-stimulatory receptor is a fragment of the co-stimulatory receptor that retains its function to mediate a co-stimulatory signal and stimulate the immune effector cell. In some embodiments, a functional fragment of a co-stimulatory receptor retains the co-stimulatory domain of the co-stimulatory receptor. In some embodiments, the co-stimulatory domain is the cytoplasmic domain of the co-stimulatory receptor. In some embodiments, signals from co-stimulatory receptors of immune effector cells (e.g., T cells) lower the activation threshold for the immune effector cells. In some embodiments, signals from co-stimulatory receptors of T cells lead to the augmentation of TCR signaling events necessary for efficient cytokine production (via augmented transcriptional activity and messenger RNA stabilization), cell cycle progression, survival, regulation of metabolism and T cell responses.

A “co-stimulatory ligand,” as used herein and understood in the art, refers to a molecule that specifically binds a cognate co-stimulatory receptor on an immune effector cell, thereby providing a signal which, in addition to the primary signal provided by the stimulatory molecule, mediates a response in the immune effector cell, including, but not limited to, proliferation, activation, differentiation, and the like. The co-stimulatory ligand can be present on an APC (e.g., a dendritic cell). Co-stimulatory ligands include, but are not limited to, CD58, CD70, CD83, CD80, CD86, CD137L (4-1BBL), CD252 (OX40L), CD275 (ICOS-L), CD54 (ICAM-1), CD49a, CD112 (PVRL2), CD150 (SLAM), CD155 (PVR), CD265 (RANK), CD270 (HVEM), TL1A, CD127, IL-4R, GITR-L, TIM-4, CD153 (CD30L), CD48, CD160, CD200R (OX2R), and CD44. A “receptor-binding fragment” of a co-stimulatory ligand refers to a fragment of the ligand that retains its capacity to bind its receptor.

### Exemplary LACO-Stim Fusion Proteins

Accordingly, provided herein are fusion proteins comprising a first domain that activates an APC and a second domain that activates an immune effector cell, wherein the first domain comprises an anti-CD40 antibody or antigen-binding fragment described herein and wherein the second domain comprises (a) a co-stimulatory receptor of the immune effector cell, or a functional fragment thereof, (b) a ligand that binds a co-stimulatory receptor of the immune effector cell, or a receptor-binding fragment thereof, or (c) an antibody that binds a co-stimulatory receptor of the immune effector cell, or an antigen-binding fragment thereof. In some embodiments, the APC is selected from the group consisting of a dendritic cell, a macrophage, a myeloid derived suppressor cell, a monocyte, a B cell, a T cell, and a Langerhans cell. In some embodiments, the immune effector cell is selected from the group consisting of a T cell, an NK cell, an NKT cell, a macrophage, a neutrophil, and a granulocyte.

The first domain can comprise any anti-CD40 antibody or antigen-binding fragment described herein. In some embodiments, the first domain comprises a monoclonal antibody. In some embodiments the first domain comprises a chimeric antibody. In some embodiments the first domain comprises a humanized antibody. In some embodiments the first domain comprises a human antibody. In some embodiments, the first domain comprises a Fab, Fab′, F(ab′)2, Fv, scFv, (scFv)2, single chain antibody, dual variable region antibody, diabody, nanobody, or single variable region antibody. In some embodiments the first domain comprises a human antibody. In some embodiments, the first domain comprises a scFv.

The first domain can have any anti-CD40 antibody or antigen-binding fragment described herein. In some embodiments, the anti-CD40 antibody or antigen-binding fragment can be one of the followings scFv clones: 406, 409, 4011, 4012, 4021, 4025, 4034, 4037, 4044, 4045 and 4052.

In some embodiments, the second domain of fusion proteins provided herein comprises (a) a co-stimulatory receptor of the immune effector cell, or a functional fragment thereof, (b) a ligand that binds a co-stimulatory receptor of the immune effector cell, or a receptor-binding fragment thereof, or (c) an antibody that binds a co-stimulatory receptor of the immune effector cell, or an antigen-binding fragment thereof.

The immune effector cell can be selected from the group consisting of a T cell, an NK cell, an NKT cell, a macrophage, a neutrophil, and a granulocyte. In some embodiments, the second domain of fusion proteins provided herein comprises a co-stimulatory receptor of the immune effector cell, or a functional fragment thereof, wherein the immune cell is a T cell, an NK cell, an NKT cell, a macrophage, a neutrophil, or a granulocyte.

In some embodiments, the co-stimulatory receptor of the immune effector cell is selected from the group consisting of CD28, 4-1BB, ICOS, CD27, OX40, DAP10, 2B4, CD30, CD2, LIGHT, GITR, DR3, and CD43. In some embodiments, the second domain of fusion proteins provided herein comprises a functional fragment of a co-stimulatory receptor selected from the group consisting of CD28, 4-1BB, ICOS, CD27, OX40, DAP10, 2B4, CD30, CD2, LIGHT, GITR, DR3, and CD43.

In some embodiments, fusion proteins provided herein further comprise a transmembrane region. In some embodiments, the transmembrane region is derived from the same co-stimulatory receptor. In some embodiments, the transmembrane region is derived from a different co-stimulatory receptor. In some embodiments, the second domain comprises a CD28 transmembrane region and a CD28 cytoplasmic domain. In some embodiments, provided herein are fusion proteins having a first domain that comprises an anti-CD40 antibody or an antigen-binding fragment thereof, and a second domain that comprises a 4-1BB transmembrane region and a 4-1BB cytoplasmic domain.

In some embodiments, the second domain comprises a co-stimulatory ligand of the immune effector cell, or a receptor-binding fragment thereof. In some embodiments, the fusion protein comprises a first domain that comprises an anti-CD40 antibody or antigen-binding fragment thereof disclosed herein, and a second domain comprises the ligand selected from the group consisting of CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R, CD44, and receptor-binding fragments thereof. A person of ordinary skill in the art can readily determine a proper receptor-binding fragment of a ligand that retains its binding affinity toward its receptor and function to activate the receptor.

In some embodiments, the second domain of fusion proteins provided herein comprises an antibody that binds a co-stimulatory receptor of the immune effector cell, or an antigen-binding fragment thereof. The immune effector cell can be selected from the group consisting of a T cell, an NK cell, an NKT cell, a macrophage, a neutrophil, and a granulocyte. In some embodiments, the co-stimulatory receptor of the immune effector cell is selected from the group consisting of CD28, 4-1BB, ICOS, CD27, OX40, DAP10, 2B4, CD30, CD2, LIGHT, GITR, DR3, and CD43. In some embodiments, the second domain comprises an antibody that binds CD28, or an antigen-binding fragment thereof.

In some embodiments, the second domain comprises a monoclonal antibody. In some embodiments the second domain comprises a chimeric antibody. In some embodiments the second domain comprises a humanized antibody. In some embodiments the second domain comprises a human antibody. In some embodiments, the second domain comprises a Fab, Fab′, F(ab′)2, Fv, scFv, (scFv)2, single chain antibody, dual variable region antibody, diabody, nanobody, or single variable region antibody. In some embodiments the second domain comprises a human antibody. In some embodiments, the second domain comprises a scFv. In some embodiments, the second domain of the fusion proteins provided herein comprise an anti-CD28 antibody or antigen-binding fragment thereof. In some embodiments, the second domain of the fusion proteins provided herein comprise an anti-CD28 scFv. In some embodiments, the anti-CD28 antibody or antigen-binding fragment thereof comprises the antibody that is designated 1412. In some embodiments, the anti-CD28 antibody or antigen-binding fragment thereof comprises the antibody that is designated 9.3h11.

In some embodiments, the fusion proteins provide herein comprise a first antigen-binding domain that specifically binds CD40 and a second antigen-binding domain that specifically binds CD28.

In some embodiments, the first antigen-binding domain provided herein comprises one, two, three, four, five, and/or six CDRs of any one of the CDRs in Table 5. In some embodiments, the first antigen-binding domain provided herein comprises a VL comprising one, two, and/or three, LCDRs from Table 5. In some embodiments, the first antigen-binding domain provided herein comprises a VH comprising one, two, and/or three HCDRs from Table 5. In some embodiments, the first antigen-binding domain provided herein comprises one, two, and/or three LCDRs from Table 5 and one, two, and/or three HCDRs from Table 5.

TABLE 5
Amino acid sequences of CDRs of the
first antigen-binding domain

| Clone | 4025 |
|---|---|
| HCDR1 | SNWMS (SEQ ID NO: 1) |
| HCDR2 | NINQDGSEKYYVDSVKG (SEQ ID NO: 2) |
| HCDR3 | GGYRYAY (SEQ ID NO: 3) |
| LCDR1 | TGTRSDIGDYNYVS (SEQ ID NO: 4) |
| LCDR2 | DVSSRPS (SEQ ID NO: 5) |
| LCDR3 | SSYTSSSTLV (SEQ ID NO: 6) |

In some embodiments, the second antigen-binding domain provided herein comprises one, two, three, four, five, and/or six CDRs of any one of the CDRs in Table 6. In some embodiments, the second antigen-binding domain provided herein comprises a VL comprising one, two, and/or three, LCDRs from Table 6. In some embodiments, the second antigen-binding domain provided herein comprises a VH comprising one, two, and/or three HCDRs from Table 6. In some embodiments, the second antigen-binding domain provided herein comprises one, two, and/or three LCDRs from Table 6 and one, two, and/or three HCDRs from Table 6.

TABLE 6
Amino acid sequences of CDRs of the
first antigen-binding domain

| Clone | 9.3h11 |
|---|---|
| HCDR1 | DYGVH (SEQ ID NO: 7) |
| HCDR2 | VIWAGGGTNYNSALMS (SEQ ID NO: 8) |
| HCDR3 | DKGYSYYYSMDY (SEQ ID NO: 9) |
| LCDR1 | RASESVEYYVTSLMQ (SEQ ID NO: 10) |
| LCDR2 | AASNVES (SEQ ID NO: 11) |
| LCDR3 | QQSRKVPYT (SEQ ID NO: 12) |

The specific CDR sequences defined herein are generally based on Kabat definitions. However, it is understood that reference to a heavy chain CDR or CDRs and/or a light chain CDR or CDRs of a specific antibody encompass all CDR definitions as known to those of skill in the art.

In some embodiments, the VH of the first antigen-binding domain comprising at least 85%, at least 90%, at least 95%, at least 98%, or 100% sequence identity to the amino acid sequence as set forth in any of SEQ ID NO: 44, and the VL of the first antigen-binding domain comprising at least 85%, at least 90%, at least 95%, at least 98%, or 100% sequence identity to the amino acid sequence as set forth in any of SEQ ID NO: 48. In some embodiments, the VH of the first antigen-binding domain comprising the amino acid sequence as set forth in any of SEQ ID NO: 44, and the VL of the first antigen-binding domain comprising the amino acid sequence as set forth in any of SEQ ID NO: 48.

In some embodiments, the VH of the second antigen-binding domain comprising at least 85%, at least 90%, at least 95%, at least 98%, or 100% sequence identity to the amino acid sequence as set forth in any of SEQ ID NO: 183, and the VL of the second antigen-binding domain t comprising at least 85%, at least 90%, at least 95%, at least 98%, or 100% sequence identity to the amino acid sequence as set forth in any of SEQ ID NO: 184. In some embodiments, the VH of the second antigen-binding domain comprising the amino acid sequence as set forth in any of SEQ ID NO: 183, and the VL of the second antigen-binding domain comprising the amino acid sequence as set forth in any of SEQ ID NO: 184.

In some embodiments, the antigen-binding fragment provided herein can be a single domain antibody (sdAb), a heavy chain antibody (HCAb), a Fab, a Fab′, a F(ab′)2, a Fv, a single-chain variable fragment (scFv), or a (scFv)2. In some embodiments, the first antigen-binding domain and/or the second antigen-binding domain comprises a Fab, a Fab′, a F(ab′)2, a Fv, a scFv, or a (scFv)2. In some embodiments, the first antigen-binding domain and/or the second antigen-binding domain is a Fab. In some embodiments, the first antigen-binding domain and/or the second antigen-binding domain is a Fab′. In some embodiments, the first antigen-binding domain and/or the second antigen-binding domain is a F(ab′)2. In some embodiments, the first antigen-binding domain and/or the second antigen-binding domain is a Fv. In some embodiments, the first antigen-binding domain and/or the second antigen-binding domain a scFv. In some embodiments, the first antigen-binding domain and/or the second antigen-binding domain is a disulfide-linked scFv [(scFv)2]. In some embodiments, the first antigen-binding domain and/or the second antigen-binding domain is a diabody (dAb).

In some embodiments, the first antigen-binding domain and/or the second antigen-binding domain comprises a scFv. In some embodiments, the scFv provided herein comprises a VH and a VL linked by a peptide linker. Unless otherwise specified, as used herein, an scFv can have the VL and VH described in any order (e.g., relative to the N-terminus and C-terminus of the polypeptide), and the scFv may include VL-linker-VH or may include VH-linker-VL. The peptide linker between VH and VL can comprise the amino acid sequence as set forth in SEQ ID NO: 97.

In some embodiments, the first antigen-binding domain is at least 85%, 90%, 95%, 98%, 99% or 100% identical to the amino acid sequence as set forth in SEQ ID NO: 52. In some embodiments, the first antigen-binding domain comprises the amino acid sequence as set forth in SEQ ID NO: 52. In some embodiments, the second antigen-binding domain is at least 85%, 90%, 95%, 98%, 99% or 100% identical to the amino acid sequence as set forth in SEQ ID NO: 185. In some embodiments, the second antigen-binding domain comprises the amino acid sequence as set forth in SEQ ID NO: 185.

In some embodiments, the first antigen-binding domain and/or the second antigen-binding domain comprises an antibody or an antigen-binding fragment thereof. In some embodiments, the antibody is an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, the first antigen-binding domain and/or the second antigen-binding domain comprises a humanized antibody or antigen-binding fragment. Various methods for generating humanized antibodies are known in the art. Methods are known in the art for achieving high affinity binding with humanized antibodies. A non-limiting example of such a method is hypermutation of the variable region and selection of the cells expressing such high affinity antibodies (affinity maturation). In addition to the use of display libraries, the specified antigen (e.g., recombinant CD40/CD28 or an epitope thereof) can be used to immunize a non-human animal, e.g., a rodent. In certain embodiments, rodent antigen-binding fragments (e.g., mouse antigen-binding fragments) can be generated and isolated using methods known in the art and/or disclosed herein. In some embodiments, a mouse can be immunized with an antigen (e.g., recombinant CD40/CD28 or an epitope thereof).

In some embodiments, the first domain and the second domain of the fusion protein bind to human CD40 and human CD28 with high affinity, respectively. For example, the affinity can be a KD of 10⁻⁷ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, 10⁻¹¹ M or less, 5×10⁻¹² M or less, 10⁻¹² M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, 10⁻⁸ M to 10⁻⁷ M, 10⁻¹⁰ M to 10⁻⁸ M, 10⁻⁹ M to 10⁻⁸ M, 10⁻¹¹ M to 10⁻⁹ M, or 10⁻¹⁰ M to 10⁻⁹ M. In some embodiments, the first domain and the second domain of the fusion protein bind to human CD40 and human CD28 respectively with a KD of 10⁻¹¹ M to 5×10⁻⁹ M. In some embodiments, the first domain of the fusion protein binds to soluble human CD40. In some embodiments, the second domain of the fusion protein binds to soluble human CD28. For soluble human CD40 or CD28, the binding affinity can be determined by BLI, with a KD of 10⁻⁷ M or less, 10⁻⁸ M or less, 5×10⁻⁹ M or less, 10⁻⁹ M or less, 5×10⁻¹⁰ M or less, 10⁻¹° M or less, 5×10⁻¹¹ M or less, 10⁻¹¹ M or less, 5×10⁻¹² M or less, 10⁻¹² M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, 10⁻⁸ M to 10⁻⁷, 10⁻¹⁰ M to 10⁻⁸ M, 10⁻⁹ M to 10⁻⁸ M, 10⁻¹¹ M to 10⁻⁹ M, or 10⁻¹⁰ M to 10⁻⁹ M. For In some embodiments, the first domain of the fusion protein binds to bound (e.g., cell membrane bound) human CD40, such as on activated human T cells. In some embodiments, the second domain binds to bound (e.g., cell membrane bound) human CD28, such as on activated human T cells. For bound human CD40 or CD28, the binding affinity can be determined by flow cytometry and Scatchard plot with a KD of 10⁻⁷ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, 10⁻¹¹ M or less, 5×10⁻¹² M or less, 10⁻¹² M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, 10⁻⁸ M to 10⁻⁷ M, 10⁻¹⁰ M to 10⁻⁸ M, 10⁻⁹ M to 10⁻⁸ M, 10⁻¹¹ M to 10⁻⁹ M, or 10⁻¹⁰ M to 10⁻⁹ M; or with an EC50 of 10 μg/mL or less, 5 μg/mL or less, 1 μg/mL or less, 0.9 μg/mL or less, 0.8 μg/mL or less, 0.7 μg/mL or less, 0.6 μg/mL or less, 0.5 μg/mL or less, 0.4 μg/mL or less, 0.3 μg/mL or less, 0.2 μg/mL or less, 0.1 μg/mL or less, 0.05 μg/mL or less, or 0.01 μg/mL or less.

In some embodiments, the fusion protein provided herein are isolated. In some embodiments, the fusion protein provided herein are substantially pure.

In some embodiments, the fusion protein provided herein comprises an amino acid sequence that is at least 85%, 90%, 95%, 98%, or 99% identical to the amino acid sequence as set forth in any one of SEQ ID NO: 155-165 or SEQ ID NO: 186. In some embodiments, the fusion protein provided herein comprises the amino acid sequence as set forth in any one of SEQ ID NO: 155-165 or SEQ ID NO: 186.

### Polynucleotides and Vectors

Also provided herein are polynucleotides that encode a polypeptide (e.g., an anti-CD40 antibody or antigen-binding fragment, or a LACO molecule) described herein. The term “polynucleotide that encode a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA can be cDNA, genomic DNA, or synthetic DNA, and can be double-stranded or single-stranded. Single stranded DNA can be the coding strand or non-coding (anti-sense) strand. The polynucleotides of the disclosure can be mRNA. Polynucleotides provided herein can be prepared, manipulated, and/or expressed using any of a variety of well-established techniques known and available in the art.

The present disclosure also provides variants of the polynucleotides described herein, wherein the variants encode, for example, fragments, analogs, and/or derivatives of an anti-CD40 antibody or antigen-binding fragment disclosed herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an anti-CD40 antibody or antigen-binding fragment described herein.

The present disclosure also provides variants of the polynucleotides described herein, wherein the variants encode, for example, fragments, analogs, and/or derivatives of a LACO molecule disclosed herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding a LACO molecule described herein.

Vectors and cells comprising the polynucleotides described herein are also provided. In some embodiments, provided herein are vectors comprising a polynucleotide provided herein. The vectors can be expression vectors. In some embodiments, vectors provided herein comprise a polynucleotide encoding an anti-CD40 antibody or antigen-binding fragment described herein. In some embodiments, vectors provided herein comprise a polynucleotide encoding a polypeptide that is part of an anti-CD40 antibody or antigen-binding fragment described herein. In some embodiments, vectors provided herein comprise a polynucleotide encoding a fusion protein described herein. In some embodiments, vectors provided herein comprise a polynucleotide encoding a LACO molecule described herein.

A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

The present disclosure also provides host cells comprising the polypeptides described herein, polynucleotides encoding polypeptides described herein, or vectors comprising such polynucleotides. In some embodiments, provided herein are host cells comprising a vector comprising a polynucleotide disclosed herein. In some embodiments, host cells provided herein comprise a vector comprising a polynucleotide encoding an anti-CD40 antibody or antigen-binding fragment described herein or a LACO molecule described herein. In some embodiments, host cells provided herein comprise a vector comprising a polynucleotide encoding a polypeptide that is part of an anti-CD40 antibody or antigen-binding fragment described herein a LACO molecule described herein. In some embodiments, host cells provided herein comprise a polynucleotide encoding an anti-CD40 antibody or antigen-binding fragment described herein a LACO molecule described herein. In some embodiments, the cells produce the anti-CD40 antibodies or antigen-binding fragments described herein the LACO molecule described herein. In some embodiments, host cells provided herein comprise a vector comprising a polynucleotide encoding a fusion protein described herein. In some embodiments, host cells provided herein comprise a polynucleotide encoding a fusion protein described herein. In some embodiments, the cells produce the fusion protein described herein. In some embodiments, host cells provided herein comprise a vector comprising a polynucleotide encoding the LACO molecule described herein. In some embodiments, host cells provided herein comprise a polynucleotide encoding the LACO molecule described herein. In some embodiments, the cells produce the LACO molecule described herein.

### Cells Recombinantly Expresses the Fusion Protein

Provided herein are cells recombinantly expressing the antigen-binding protein that specifically binds CD40 provided herein. Provided herein are cells recombinantly expressing the fusion proteins disclosed herein. Provided herein are also cells comprising the polynucleotides disclosed herein. In some embodiments, provided herein are cells comprising the vectors disclosed herein.

In some embodiments, the cell expressing the fusion proteins disclosed herein is a genetically engineered cell. In some embodiments, the cell expressing the fusion proteins disclosed herein is a genetically engineered immune effector cell.

In some embodiments, the cell expressing the fusion proteins disclosed herein comprises an immune effector cell. is selected from the group consisting of a T cell, an NK cell, an NKT cell, a macrophage, a neutrophil, and a granulocyte. In some embodiments, the cell provided herein is a T cell. In some embodiments, the cell provided herein is an NK cell. In some embodiments, the cell provided herein is an NKT cell. In some embodiments, the cell provided herein is a macrophage. In some embodiments, the cell provided herein is a neutrophil. In some embodiments, the cell provided herein is a granulocyte. In some embodiments, the genetically engineered immune effector cells provided herein are isolated. In some embodiments, the genetically engineered immune effector cells provided herein are substantially pure.

In some embodiments, the immune effector cell provided herein is a T cell. The T cell can be a cytotoxic T cell, a helper T cell, or a gamma delta T, a CD4+/CD8+ double positive T cell, a CD4+T cell, a CD8+ T cell, a CD4/CD8 double negative T cell, a CD3+ T cell, a naive T cell, an effector T cell, a cytotoxic T cell, a helper T cell, a memory T cell, a regulator T cell, a Th0 cell, a Th1 cell, a Th2 cell, a Th3 (Treg) cell, a Th9 cell, a Th17 cell, a Thap helper cell, a Tfh cell, a stem memory TSCM cell, a central memory TCM cell, an effector memory TEM cell, an effector memory TEMRA cell, or a gamma delta T cell. In some embodiments, the T cell is a cytotoxic T cell. In some embodiments, the genetically engineered T cells provided herein are isolated. In some embodiments, the genetically engineered T cells provided herein are substantially pure.

In some embodiments, genetically engineered cells provided herein are derived from cells isolated from a subject. As used herein, a genetically engineered cell that is “derived from” a source cell means that the genetically engineered cell is obtained by taking the source cell and genetically manipulating the source cell. The source cell can be from a natural source. For example, the source cell can be a primary cell isolated from a subject. The subject can be an animal or a human. The source cell can also be a cell that has undergone passages or genetically manipulation in vitro.

In some embodiments, genetically engineered cells provided herein are derived from cells isolated from a human. Immune effector cells (e.g., T cells) can be obtained from many sources, including peripheral blood mononuclear cells, bone marrow, lymph node tissue, cord blood, thymus tissue, tissue from a site of infection, ascites, pleural effusion, spleen tissue, and tumors. In certain embodiments, T cell lines available in the art can be used. In some embodiments, genetically engineered cells provided herein are derived from cells isolated from peripheral blood. In some embodiments, genetically engineered cells provided herein are derived from cells isolated from bone marrow. In some embodiments, genetically engineered cells provided herein are derived from cells isolated from peripheral blood mononuclear cells (PBMC).

In some embodiments, genetically engineered cells provided herein are derived from cells differentiated in vitro from a stem or progenitor cell. In some embodiments, the stem or progenitor cell is selected from the group consisting of a T cell progenitor cell, a hematopoietic stem and progenitor cell, a hematopoietic multipotent progenitor cell, an embryonic stem cell, and an induced pluripotent cell. In some embodiments, genetically engineered cells provided herein are derived from cells differentiated in vitro from a T cell progenitor cell. In some embodiments, genetically engineered cells provided herein are derived from cells differentiated in vitro from a hematopoietic stem and progenitor cell. In some embodiments, genetically engineered cells provided herein are derived from cells differentiated in vitro from a hematopoietic multipotent progenitor cell. In some embodiments, genetically engineered cells provided herein are derived from cells differentiated in vitro from an embryonic stem cell. In some embodiments, genetically engineered cells provided herein are derived from cells differentiated in vitro from an induced pluripotent cell.

In some embodiments, provided herein are a population of the genetically engineered cells disclosed herein. The population of cells can be a homogenous population of cells. The population of cells can be a heterogeneous population of cells. In some embodiments, the population of cells can be a heterogeneous population of cells comprising any combination of the cells disclosed herein. In some embodiments, the population of genetically engineered cells provided herein are derived from tumor-infiltrating lymphocytes (TIL). In some embodiments, the population of genetically engineered cells provided herein are derived from peripheral blood mononuclear cells (PBMC). In some embodiments, the population of genetically engineered cells provided herein are derived from peripheral blood leukocytes (PBL). In some embodiments, the population of genetically engineered cells provided herein are derived from tumor infiltrating lymphocytes (TIL). In some embodiments, the population of genetically engineered cells provided herein are derived from marrow infiltrate lymphocytes (MILs). In some embodiments, the population of genetically engineered cells provided herein are derived from cytokine-induced killer cells (CIK). In some embodiments, the population of genetically engineered cells provided herein are derived from lymphokine-activated killer cells (LAK).

In some embodiments, the genetically engineered immune effector cells provided herein further recombinantly express a chimeric antigen receptor (CAR), a T cell receptor (TCR) or a Bi-specific T-cell engager (BiTE). In some embodiments, the genetically engineered cells disclosed herein further express a CAR. In some embodiments, the genetically engineered cells disclosed herein further express a TCR. In some embodiments, the genetically engineered cells disclosed herein further express a BiTE. In some embodiments, the genetically engineered immune effector cells provided herein further comprise a polynucleotide that encodes a CAR, a TCR or a BiTE (CAR/TCR/BiTE). In some embodiments, the genetically engineered immune effector cells provided herein further comprise a polynucleotide that encodes a CAR. In some embodiments, the genetically engineered immune effector cells provided herein further comprise a polynucleotide that encodes a TCR. In some embodiments, the genetically engineered immune effector cells provided herein further comprise a polynucleotide that encodes a BiTE. In some embodiments, the CAR, TCR or BiTE binds a tumor antigen or a viral antigen.

In some embodiments, the genetically engineered immune effector cells provided herein further expresses a CAR or comprises a polynucleotide that encodes a CAR. The CAR can be any CAR disclosed herein or otherwise known in the art. In some embodiments, the CAR comprises an antigen-binding domain that specifically binds a tumor antigen. As such, in some embodiments, provided herein are also genetically engineered cells expressing a fusion protein disclosed herein and a CAR. In some embodiments, genetically engineered cells provided herein comprise a polynucleotide that comprises a first fragment encoding a fusion protein, and a second fragment encoding a CAR. In some embodiments, genetically engineered cells provided herein comprise a first polynucleotide encoding a fusion protein provided herein, and a second polynucleotide encoding a CAR.

In some embodiments, the CAR, TCR, or BiTE provided herein include a target-binding domain that binds an antigen. In some embodiments, the antigen is a viral antigen. In some embodiments, the viral antigen is EBV. In some embodiments, the viral antigen is HPV. In some embodiments, the viral antigen is HIV. It is understood that these or other viral antigens can be utilized for targeting by a CAR, TCR, or BiTE disclosed herein.

In some embodiments, the CAR, TCR, or BiTE provided herein include a target-binding domain that binds a cancer antigen or a tumor antigen. Any suitable cancer antigen or tumor antigen can be chosen based on the type of cancer exhibited by a subject (cancer patient) to be treated. It is understood that the selected cancer antigen is expressed in a manner such that the cancer antigen is accessible for binding. Generally, the cancer antigen to be targeted by a cell expressing a CAR, TCR, or BiTE is expressed on the cell surface of a cancer cell. However, it is understood that any cancer antigen that is accessible for binding is suitable for targeting.

Suitable antigens include, but are not limited to, TSHR, CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family members; B cell maturation antigen (BCMA); Tn antigen ((Tn Ag) or (GalNAca-Ser/Thr)); prostate-specific membrane antigen (PSMA); receptor tyrosine kinase-like orphan receptor 1 (RORI); Fms-like tyrosine kinase 3 (FLT3); tumor-associated glycoprotein 72 (TAG72); CD38; CD44v6; carcinoembryonic antigen (CEA); epithelial cell adhesion molecule (EPCAM); B7H3 (CD276); KIT (CD117); interleukin-13 receptor subunit a-2; mesothelin; interleukin 11 receptor α (IL-l lRa); prostate stem cell antigen (PSCA); protease serine 21; vascular endothelial growth factor receptor 2 (VEGFR2); Lewis(Y) antigen; CD24; platelet-derived growth factor receptor R (PDGFR-beta); stage-specific embryonic antigen-4 (SSEA-4); CD20; folate receptor u; receptor tyrosine-protein kinase ERBB2 (Her2/neu); mucin 1, cell surface associated (MUC1); epidermal growth factor receptor (EGFR); neural cell adhesion molecule (NCAM); prostase; prostatic acid phosphatase (PAP); elongation factor 2 mutated (ELF2 M); Ephrin B2; fibroblast activation protein α (FAP); insulin-like growth factor 1 receptor (IGF-I receptor); carbonic anhydrase IX (CAIX); proteasome (Prosome, Macropain) subunit, β Type, 9 (LMP2); glycoprotein 100 (gp100); oncogene polypeptide composed of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); folate receptor R; tumor endothelial marker 1 (TEMi/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); polysialic acid; placenta-specific 1 (PLAC1); hexasaccharide portion of globoH glycoceramide (GloboH); mammary gland differentiation antigen (NY-BR-1); uroplakin 2 (UPK2); hepatitis A virus cellular receptor 1 (HAVCR1); adrenoceptor β3 (ADRB3); pannexin 3 (PANX3); G protein-coupled receptor 20 (GPR20); lymphocyte antigen 6 complex, locus K9 (LY6K); olfactory receptor 51E2 (OR51E2); TCR gamma alternate reading frame protein (TARP); Wilms tumor protein (WTl); cancer/testis antigen 1 (NY-ESO-1); cancer/testis antigen 2 (LAGE-la); melanoma-associated antigen 1 (MAGE-A1); ETS translocation-variant gene 6, located on chromosome 12p (ETV6-AML); sperm protein 17 (SPA 17); X antigen family, member IA (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1; melanoma antigen recognized by T cells 1; rat sarcoma (Ras) mutant; human telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-acetyl glucosaminyl-transferase V (NA17); paired box protein Pax-3 (PAX3); androgen receptor; Cyclin B1; v-myc avian myelocytomatosis viral oncogene neuroblastoma derived homolog (MYCN); Ras homolog family member C (RhoC); tyrosinase-related protein 2 (TRP-2); cytochrome P450 1B 1 (CYP1B1); CCCTC-binding factor (zinc finger protein)-Like, squamous cell carcinoma antigen recognized by T cells 3 (SART3); paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); receptor for advanced glycation endproducts (RAGE-1); renal ubiquitous 1 (RUl); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; leukocyte-associated immunoglobulin-like receptor 1 (LAIRl); Fc fragment of IgA receptor (FCAR or CD89); leukocyte immunoglobulin-like receptor subfamily A member 2 (LILRA2); CD300 molecule-like family member f (CD300LF); C-type lectin domain family 12 member A (CLECl2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin X-like polypeptide 1 (IGLL1). It is understood that these or other cancer antigens can be utilized for targeting by a CAR, TCR, or BiTE disclosed herein.

In some embodiments, the genetically engineered immune effector cells provided herein further comprise a polynucleotide that encodes a CAR, TCR, or BiTE that binds a cancer antigen or tumor antigen. In some embodiments, the genetically engineered immune effector cells provided herein further recombinantly express a CAR, TCR, or BiTE that binds a cancer antigen or tumor antigen. In some embodiments, the cancer antigen or tumor antigen is selected from the group consisting of CD70, HER2, NY-ESO-1, CD19, CD20, CD22, PSMA, c-Met, GPC3, IL13ra2, EGFR, CD123, CD7, GD2, PSCA, EBV16-E7, H3.3, EGFRvIII, BCMA, Mesothelin, GPRC5D, GCC, GUCY2C, Claudin 18.2, RORI, B7H3, CDH17 and DLL3.

In some embodiments, the genetically engineered immune effector cells provided herein comprise a polynucleotide that comprises a first fragment encoding a CAR and a second fragment encoding a fusion protein provided herein. In some embodiments, the genetically engineered immune effector cells provided herein comprise a polynucleotide that comprises a first fragment encoding a TCR and a second fragment encoding a fusion protein. In some embodiments, the polynucleotide has the first fragment and the second fragment from N-terminus to the C-terminus. In some embodiments, the polynucleotide has the second fragment and the first fragment from N-terminus to the C-terminus. In some embodiments, the cells provided herein comprise a polynucleotide having a first fragment encoding a CAR or a TCR provided herein and a second fragment that encodes a fusion protein disclosed herein. The first fragment and the second fragment can be linked by a nucleotide sequence encoding a linker. The linker can be a self-cleaving linker. In some embodiments, the first and second fragment are linked by a nucleotide sequence encoding a 2A peptide. In some embodiments, the linker is an F2A peptide (e.g.: SEQ ID NO: 101). In some embodiments, the 2A linker is a T2A peptide (e.g.: SEQ ID NO: 245). In some embodiments, the linker is a Furin-GS2-T2A peptide (e.g.: SEQ ID NO: 215). In some embodiments, the first fragment (CAR/TCR-encoding) is located at the 5′ end of the second fragment (fusion protein-encoding). In some embodiments, the first fragment (CAR/TCR-encoding) is located at the 3′ end of the second fragment (fusion protein encoding).

In some embodiments, cells co-expressing the first and second fragments may comprise the amino acid sequences as set forth in any one of the groups comprising SEQ ID NOs: 155-165 and SEQ ID NO: 186 and/or the nucleotide encoding the amino acid sequence thereof.

### CARs

The genetically engineered immune effector cells (e.g., T cells) provided herein can be used in cancer treatment. In some embodiments, provided herein is a genetically engineered T cell that expresses the fusion protein disclosed herein. In some embodiments, provided herein is a genetically engineered T cell that comprises the polynucleotide disclosed herein. In some embodiments, provided herein is a CAR-T cell.

In some embodiments, the fusion proteins provided herein can be co-expressed with a CAR in an immune effector cell. In some embodiments, a fusion protein provided herein can be conjugated to a CAR. CARs retarget immune effector cells (e.g., T cells) to tumor surface antigens (Sadelain et al., Nat. Rev. Cancer. 3(1):35-45 (2003); Sadelain et al., Cancer Discovery 3(4):388-398 (2013)). CARs are engineered receptors that provide both antigen binding and immune effector cell activation functions. CARs can be used to graft the specificity of an antibody, such as a monoclonal antibody, onto an immune effector cell such as a T cell, a NK cell, or a macrophage. First-generation receptors link an antibody-derived tumor-binding element, such as an scFv, that is responsible for antigen recognition to either CD3zeta or Fc receptor signaling domains, which trigger T-cell activation. The advent of second-generation CARs, which combine activating and costimulatory signaling domains, has led to encouraging results in patients with chemorefractory B-cell malignancies (Brentjens et al., Science Translational Medicine 5(177):177ra38 (2013); Brentjens et al., Blood 118(18):4817-4828 (2011); Davila et al., Science Translational Medicine 6(224):224ra25 (2014); Grupp et al., N. Engl. J Med. 368(16):1509-1518 (2013); Kalos et al., Science Translational Medicine 3(95):95ra73 (2011)). The extracellular antigen-binding domain of a CAR is usually derived from a monoclonal antibody (mAb) or from receptors or their ligands. Antigen binding by the CARs triggers phosphorylation of immunoreceptor tyrosine-based activation motifs (ITAMs) in the intracellular domain, initiating a signaling cascade required for cytolysis induction, cytokine secretion, and proliferation.

In some embodiments, a fusion protein provided herein can be conjugated to CAR that has an antigen binding domain that binds to a cancer antigen. In some embodiments, the CAR can be a “first generation,” “second generation” or “third generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J Immunol. 173:2143-2150 (2004); Sadelain et al., Curr. Opin. Immunol. 21(2):215-223 (2009); Hollyman et al., J. Immunother. 32:169-180 (2009)).

In addition to T cells, CAR can be engineered into other types of immune effector cells, such as NK cells, NKT cells, macrophages, or granulocytes. In some embodiments, the engineered cell is a NK cell. CARs provided herein can retarget NK cells to tumor surface antigens (see e.g., Hu et al. Acta Pharmacol Sin 39, 167-176 (2018)). CAR-NK cells can use the first generation of CAR constructs that contain CD3ζ as an intracellular signaling domain or the second generation of CAR constructs that express a second signaling domain (e.g., CD28, 4-1BB) in conjunction with CD3. In general, the second generation of CARs in NK cells is more active than first-generation CARs. In some embodiments, CAR constructs are based on the activating features of NK cells. For example, DNAX-activation protein 12 (DAP12) is known to activate signaling for NK cells.

CARs provided herein can include a target-binding domain as disclosed above. In some embodiments, fusion proteins disclosed herein can be co-expressed with a CAR. In some embodiments, fusion proteins disclosed herein can be conjugated to a CAR. In some embodiments, genetically engineered immune effector cells provided herein further comprise a polynucleotide encoding a CAR. In some embodiments, genetically engineered immune effector cells provided herein further recombinantly express a CAR.

In some embodiments, CARs provided herein include an antigen-binding domain. Suitable antigens include tumor antigens and/or viral antigens. The tumor antigens may comprise but are not limited to, TSHR, CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family members; B cell maturation antigen (BCMA); Tn antigen ((Tn Ag) or (GalNAca-Ser/Thr)); prostate-specific membrane antigen (PSMA); receptor tyrosine kinase-like orphan receptor 1 (RORI); Fms-like tyrosine kinase 3 (FLT3); tumor-associated glycoprotein 72 (TAG72); CD38; CD44v6; carcinoembryonic antigen (CEA); epithelial cell adhesion molecule (EPCAM); B7H3 (CD276); KIT (CD117); interleukin-13 receptor subunit α-2; mesothelin; interleukin 11 receptor a (IL-1 lRa); prostate stem cell antigen (PSCA); protease serine 21; vascular endothelial growth factor receptor 2 (VEGFR2); Lewis(Y) antigen; CD24; platelet-derived growth factor receptor R (PDGFR-beta); stage-specific embryonic antigen-4 (SSEA-4); CD20; folate receptor u; receptor tyrosine-protein kinase ERBB2 (Her2/neu); mucin 1, cell surface associated (MUC1); epidermal growth factor receptor (EGFR); neural cell adhesion molecule (NCAM); prostase; prostatic acid phosphatase (PAP); elongation factor 2 mutated (ELF2 M); Ephrin B2; fibroblast activation protein α (FAP); insulin-like growth factor 1 receptor (IGF-I receptor); carbonic anhydrase IX (CAIX); proteasome (Prosome, Macropain) subunit, β Type, 9 (LMP2); glycoprotein 100 (gp100); oncogene polypeptide composed of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); folate receptor $; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); polysialic acid; placenta-specific 1 (PLAC1); hexasaccharide portion of globoH glycoceramide (GloboH); mammary gland differentiation antigen (NY-BR-1); uroplakin 2 (UPK2); hepatitis A virus cellular receptor 1 (HAVCR1); adrenoceptor β3 (ADRB3); pannexin 3 (PANX3); Gprotein-coupled receptor 20 (GPR20); lymphocyte antigen 6 complex, locus K9 (LY6K); olfactory receptor 51E2 (OR51E2); TCR gamma alternate reading frame protein (TARP); Wilms tumor protein (WTl); cancer/testis antigen 1 (NY-ESO-1); cancer/testis antigen 2 (LAGE-la); melanoma-associated antigen 1 (MAGE-A1); ETS translocation-variant gene 6, located on chromosome 12p (ETV6-AML); sperm protein 17 (SPA 17); X antigen family, member IA (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1; melanoma antigen recognized by T cells 1; rat sarcoma (Ras) mutant; human telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-acetyl glucosaminyl-transferase V (NA17); paired box protein Pax-3 (PAX3); androgen receptor; Cyclin Bi; v-myc avian myelocytomatosis viral oncogene neuroblastoma derived homolog (MYCN); Ras homolog family member C (RhoC); tyrosinase-related protein 2 (TRP-2); cytochrome P450 1B 1 (CYP1B1); CCCTC-binding factor (zinc finger protein)-Like, squamous cell carcinoma antigen recognized by T cells 3 (SART3); paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); receptor for advanced glycation endproducts (RAGE-1); renal ubiquitous 1 (RUl); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; leukocyte-associated immunoglobulin-like receptor 1 (LAIRl); Fc fragment of IgA receptor (FCAR or CD89); leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin X-like polypeptide 1 (IGLL1). The viral antigen may comprise but are not limited to HPV, EBV, and HIV.

In some embodiments, the CAR may comprise the antigen-binding domain, and the antigen may be selected from CD70, Her2, NY-ESO-1, CD19, CD20, CD22, PSMA, c-Met, GPC3, IL13ra2, EGFR, CD123, CD7, GD2, PSCA, EBV16-E7, H3.3, EGFRvIII, BCMA, Mesothelin, GPRC5D, GCC, GUCY2C, Claudin 18.2, RORI, B7H3, DLL3, and CDH17.

In some embodiments, the CAR comprises an antigen-binding domain, a transmembrane domain and a cytoplasmic domain.

In some embodiments, the CAR targets CD70. The present application also provides an anti-CD70 antibody or antigen-binding fragments thereof that specifically bind CD70 (e.g., human CD70). In some embodiments, the anti-CD70 antibody or antigen-binding fragments thereof provided herein comprises a light chain CDR1 (LCDR1), a light chain CDR2 (LCDR2), a light chain CDR3 (LCDR3) having the amino acid sequence of SEQ ID NOs: 105, 106, and 107 respectively, or a variant thereof having up to about 5 amino acid substitutions, additions, and/or deletions in the LCDRs; and/or a heavy chain variable region (VH) comprising a heavy chain CDR1 (HCDR1), a heavy chain CDR2 (HCDR2), a heavy chain CDR3 (HCDR3) having the amino acid sequence of SEQ ID NOs: 102, 103, and 104 respectively; or a variant thereof having up to about 5 amino acid substitutions, additions, and/or deletions in the HCDRs. In some embodiments, the anti-CD70 antibody or antigen-binding fragments thereof provided herein comprises a VL having at least 85%, at least 90%, at least 95%, at least 98%, or 100% sequence identity to an amino acid sequence of SEQ ID NO: 109, and/or a VH having at least 85%, at least 90%, at least 95%, at least 98%, or 100% sequence identity to an amino acid sequence of SEQ ID NO: 108. In some embodiments, the anti-CD70 antibody or antigen-binding fragments thereof provided herein comprises a scFv having at least 85%, at least 90%, at least 95%, at least 98%, or 100% sequence identity to an amino acid sequence of the amino acid sequence as set forth in SEQ ID NO: 110.

The present application also provides CARs target CD70. The CARs target CD70 comprises an antigen-binding domain that specifically binds to CD70. In some embodiments, the antigen-binding domain of the CAR targets CD70 comprises an anti-CD70 antibody or antigen-binding fragments thereof. In some embodiments, the antigen-binding domain of the CAR targets CD70 comprises the CD70 antibody or antigen-binding fragments thereof provided herein. In some embodiments, the antigen-binding domain of the CAR targets CD70 comprises a CD70 receptor. In some embodiments, the antigen-binding domain of the CAR targets CD70 comprises CD27 (referred as CD27 CAR in the present application). In some embodiments, the antigen-binding domain of the CAR targets CD70 comprises full length of CD27 or a fragment derived from CD27 that can specifically bind to CD70. Also provided herein are polynucleotides encoding the anti-CD70 antibody or antigen-binding fragments thereof provided herein. Vectors and cells comprising the polynucleotides encoding the anti-CD70 antibody or antigen-binding fragments thereof provided herein are also provided.

A transmembrane domain derived from a protein or polypeptide means that the transmembrane domain comprises the entire transmembrane region of the protein or polypeptide, or a fragment thereof. In some embodiments, the transmembrane domain of the CAR comprises a transmembrane domain derived from CD8, CD28, CD3ζ, CD4, 4-1BB, OX40, ICOS, CTLA-4, PD-1, LAG-3, 2B4, BTLA, TCR α chain, TCR R chain, or TCR ζ chain, CD3ε, CD45, CD5, CD8, CD9, CD16, CD22, CD33, CD37, CD64, CD80, CD86, CD134, or CD154. In some embodiments, the transmembrane domain of the CAR comprises CD8 transmembrane region.

A signaling domain derived from a protein or polypeptide refers to the domain of the protein or polypeptide that is responsible for activating the immune effector cell (e.g., a T cell), or a fragment thereof that retains its activation function. In some embodiments, the cytoplasmic domain of the CAR comprises a signaling domain derived from CD3ζ, FcRγ, FcγRIIa, FcRβ, CD3γ, CD3δ, CD3ε, CD5, CD22, CD79a, CD79b, DAP10, DAP12, or any combination thereof.

A co-stimulatory domain derived from a protein or polypeptide refers to the domain of the protein or polypeptide that is responsible for providing increased activation of an immune effector cell (e.g., T cell), or a fragment thereof that retains its activation function. In some embodiments, the cytoplasmic domain of the CAR further comprises a co-stimulatory domain derived from CD28, 4-1BB (CD137), OX40, ICOS, DAP10, 2B4, CD27, CD30, CD40, CD2, CD7, LIGHT, GITR, TLR, DR3, CD43, or any combination thereof. In some embodiments, the cytoplasmic domain of the CAR comprises a CD3ζ signaling domain and a 4-1BB co-stimulatory domain.

In some embodiments, a CAR can also comprise a spacer region or sequence that links the domains of the CAR to each other. For example, a spacer can be included between a signal peptide and an antigen binding domain, between the antigen binding domain and the transmembrane domain, between the transmembrane domain and the intracellular domain, and/or between domains within the intracellular domain, for example, between a stimulatory domain and a co-stimulatory domain. The spacer region can be flexible enough to allow interactions of various domains with other polypeptides, for example, to allow the antigen binding domain to have flexibility in orientation in order to facilitate antigen recognition. The spacer region can be, for example, the hinge region from an IgG, the CH2CH3 (constant) region of an immunoglobulin, and/or portions of CD3 (cluster of differentiation 3) or some other sequence suitable as a spacer. In some embodiments, a CAR disclosed herein comprises a hinge domain that connects the antigen-binding domain and the transmembrane domain. In some embodiments, the hinge domain comprises human CD8 hinge domain. In some embodiments, the hinge domain comprises human CD28 hinge domain.

Adverse events in cell therapy, particularly during the reinfusion of cells into patients, may be minimized by transducing immune cells with a suicide gene, also known as a safety switch. These safety switches can be implemented using various mechanisms. One approach is the use of a TET-OFF/ON system, where the expression of the CAR or other therapeutic genes in CAR-immune cells (e.g., CAR-T cells) is controlled by the presence or absence of tetracycline, allowing for the regulation of gene expression based on treatment needs. Another method involves the use of an NFAT-on switch, which ensures that the CAR or therapeutic gene is only expressed when the immune cells are activated, providing targeted control of immune cell activity. Additionally, when the suicide polypeptide coded by the suicide gene is expressed at the surface of a CAR-immune cell (e.g., CAR-T cell), binding of rituximab to the R epitopes of the polypeptide causes lysis of the cell. More than one molecule of rituximab may bind per polypeptide expressed at the cell surface. Each R epitope of the polypeptide may bind a separate molecule of rituximab. Deletion of CAR-immune cells may occur in vivo, for example by administering rituximab to a patient. The decision to eliminate the transferred cells may be prompted by the detection of adverse effects in the patient, which are linked to the transferred cells, such as when toxicities reach unacceptable levels.

In some embodiments, a suicide polypeptide is expressed on the surface of the cell. In some embodiments, a suicide polypeptide is included in the CAR construct. In some embodiments, a suicide polypeptide is not part of the CAR construct.

In some embodiments, the extracellular domain of any one of the CARs disclosed herein may comprise one or more epitopes specific for (i.e., specifically recognized by) a monoclonal antibody. These epitopes are also referred to herein as mAb-specific epitopes. Exemplary mAb-specific epitopes are disclosed in International Patent Publication No. WO2016/120216, which is incorporated herein in its entirety. In these embodiments, the extracellular domain of the CARs comprises antigen binding domains that specifically bind to tumor antigen and one or more epitopes that bind to one or more monoclonal antibodies (mAbs). CARs comprising the mAb-specific epitopes can be single-chain or multi-chain.

Several epitope-monoclonal antibody couples can be used to generate CARs comprising monoclonal antibody specific epitopes; in particular, those already approved for medical use, such as CD20 epitope/rituximab as a non-limiting example. Table 7 provides exemplary mimotope sequences that can be inserted into the extracellular domains of any one of the CARs and corresponding mAb of the disclosure.

TABLE 7
Exemplary of epitopes and mimotopes

| SEQ ID | Epitope/ | | |
|---|---|---|---|
| NO | Mimotope | Sequences | mAb |
| 205 | Mimotope | CPYSNPSLC | Rituximab |
| 206 | Epitope | NSELLSLINDMPITNDQKK | Palivizumab |
| | | LMSNN | |
| 207 | Mimotope | CQFDLSTRRLKC | Cetuximab |
| 208 | Mimotope | CQYNLSSRALKC | Cetuximab |
| 209 | Mimotope | CVWQRWQKSYVC | Cetuximab |
| 210 | Mimotope | CMWDRFSRWYKC | Cetuximab |
| 211 | Epitope | SFVLNWYRMSPSNQTDKLA | Nivolumab |
| | | AFPEDR | |
| 212 | Epitope | SGTYLCGAISLAPKAQIKE | Nivolumab |
| 213 | Epitope | ELPTQGTFSNVSTNVS | QBEND-10 |
| 214 | Epitope | GQNDTSQTSSPS | Alemtuzumab |

In some embodiments, the extracellular binding domain of the CAR comprises the following sequence: V1-L-V2-L-Epitopel-L-; V1-L-V2-L-Epitopel-L-Epitope2-L-; V1-L-V2-L-Epitopel-L-Epitope2-L-Epitope3-L-; L-Epitopel-L-V1-L-V2; L-Epitopel-L-Epitope2-L-V1-L-V2; Epitopel-L-Epitope2-L-Epitope3-L-V1-L-V2; L-Epitopel-L-V1-L-V2-L-Epitope2-L; L-Epitopel-L-V1-L-V2-L-Epitope2-L-Epitope3-L-; L-Epitope 1-L-V1-L-V2-L-Epitope2-L-Epitope3-L-Epitope4-L-; L-Epitopel-L-Epitope2-L-V1-L-V2-L-Epitope3-L-; L-Epitopel-L-Epitope2-L-V1-L-V2-L-Epitope3-L-Epitope4-L-; V1-L-Epitopel-L-V2; V1-L-Epitopel-L-V2-L-Epitope2-L; V1-L-Epitopel-L-V2-L-Epitope2-L-Epitope3-L; V1-L-Epitopel-L-V2-L-Epitope2-L-Epitope3-L-Epitope4-L; L-Epitopel-L-V1-L-Epitope2-L-V2; or, L-Epitopel-L-V1-L-Epitope2-L-V2-L-Epitope3-L wherein, V1 is VL and V2 is VH or V1 is VH and V2 is VL; L is a linker comprising glycine and serine residues, and each occurrence of L in the extracellular binding domain can be identical or different to other occurrence of L in the same extracellular binding domain, and each occurrence of L is independently from the others; and, preferably comprising an amino sequence of SEQ ID NO: 97, and Epitope 1, Epitope 2 and Epitope 3 are mAb-specific epitopes and can be identical or different, optionally having the sequences of any one of table 7.

In some embodiments, the CAR comprises the amino acid of the group consisting of the amino acid sequence as set forth in SEQ ID NO: 111, 122, 195, 197, 202-204, 224, 233, and 243.

### Pharmaceutical Compositions

Provided herein are also pharmaceutical compositions comprising anti-CD40 antibodies or antigen-binding fragments disclosed herein. Provided herein are also pharmaceutical compositions comprising soluble fusion proteins disclosed herein. Provided herein are also pharmaceutical compositions comprising the genetically engineered immune effector cells disclosed herein. In some embodiments, the pharmaceutical composition comprises an effective amount of the fusion proteins disclosed herein and a pharmaceutically acceptable carrier. In some embodiments, the pharmaceutical composition comprises an effective amount of genetically engineered cells disclosed herein and a pharmaceutically acceptable carrier. In some embodiments, the pharmaceutical compositions are useful in immunotherapy. In some embodiments, the pharmaceutical compositions are useful in immuno-oncology. In some embodiments, the pharmaceutical compositions are useful in inhibiting tumor growth in a subject (e.g., a human patient). In some embodiments, the pharmaceutical compositions are useful in treating cancer in a subject (e.g., a human patient). In some embodiments, the pharmaceutical compositions are useful in treating viral infection.

In some embodiments, the pharmaceutical compositions provided herein comprise anti-CD40 antibodies or antigen-binding fragments provided herein. The anti-CD40 antibodies or antigen-binding fragments can be present at various concentrations. In some embodiments, the pharmaceutical compositions provided herein comprise soluble anti-CD40 antibodies or antigen-binding fragments provided herein at 1-1000 mg/ml. In some embodiments, the pharmaceutical compositions comprise soluble anti-CD40 antibodies or antigen-binding fragments provided herein at 10-500 mg/ml, 10-400 mg/ml, 10-300 mg/ml, 10-200 mg/ml, 10-100 mg/ml, 20-100 mg/ml, or 50-100 mg/ml. In some embodiments, the pharmaceutical compositions provided herein comprise anti-CD40 antibodies or antigen-binding fragments provided herein at about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 120 mg/ml, about 150 mg/ml, about 180 mg/ml, about 200 mg/ml, about 300 mg/ml, about 500 mg/ml, about 800 mg/ml, or about 1000 mg/ml.

In some embodiments, the pharmaceutical compositions provided herein comprise soluble fusion proteins provided herein. The fusion protein can be present at various concentrations. In some embodiments, the pharmaceutical compositions provided herein comprise soluble fusion proteins provided herein at 1-1000 mg/ml. In some embodiments, the pharmaceutical compositions comprise soluble fusion proteins provided herein at 10-500 mg/ml, 10-400 mg/ml, 10-300 mg/ml, 10-200 mg/ml, 10-100 mg/ml, 20-100 mg/ml, or 50-100 mg/ml. In some embodiments, the pharmaceutical compositions comprise soluble fusion proteins provided herein compositions provided herein comprise soluble fusion proteins provided herein at 1-1000 mg/ml. In some embodiments, the pharmaceutical compositions provided herein comprise soluble fusion proteins provided herein at about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 120 mg/ml, about 150 mg/ml, about 180 mg/ml, about 200 mg/ml, about 300 mg/ml, about 500 mg/ml, about 800 mg/ml, or about 1000 mg/ml.

The pharmaceutical compositions comprising genetically engineered cells disclosed herein can comprise a purified population of cells. Those skilled in the art can readily determine the percentage of cells in a cell population using various well-known methods, as described herein. The ranges of purity in cell populations comprising genetically modified cells provided herein can be from about 20% to about 25%, from about 25% to about 30%, from about 30% to about 35%, from about 35% to about 40%, from about 40% to about 45%, from about 45% to about 50%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. In some embodiments, the ranges of purity in cell populations comprising genetically modified cells provided herein can be from about 20% to about 30%, from about 20% to about 50%, from about 20% to about 80%, from about 20% to about 100%, from about 50% to about 80%, or from about 50% to about 100%. Dosages can be readily adjusted by those skilled in the art; for example, a decrease in purity may require an increase in dosage.

Provided herein are also kits for preparation of pharmaceutical compositions having an anti-CD40 antibody or antigen-binding fragment disclosed herein. In some embodiments, the kit further comprises a pharmaceutically acceptable excipient in one or more containers. In another embodiment, the kits can comprise an anti-CD40 antibody or antigen-binding fragment disclosed herein for administration to a subject. In specific embodiments, the kits comprise instructions regarding the preparation and/or administration of an anti-CD40 antibody or antigen-binding fragment.

Provided herein are also kits for preparation of pharmaceutical compositions having the fusion protein disclosed herein. In some embodiments, the kit further comprises a pharmaceutically acceptable excipient in one or more containers. In another embodiment, the kits can comprise fusion proteins disclosed herein for administration to a subject. In specific embodiments, the kits comprise instructions regarding the preparation and/or administration of the fusion protein.

Provided herein are also kits for preparation of cells disclosed herein. In one embodiment, the kit comprises one or more vectors for generating a genetically engineered cell, such as a T cell, that expresses a fusion protein disclosed herein. The kits can be used to generate genetically engineered cells from autologous or non-autologous cells to be administered to a compatible subject. In another embodiment, the kits can comprise cells disclosed herein for administration to a subject. In specific embodiments, the kits comprise the cells disclosed herein in one or more containers. In specific embodiments, the kits comprise instructions regarding the preparation and/or administration of the genetically engineered cells.

In some embodiments, provided herein is a pharmaceutical composition comprising antibodies, fusion proteins or cells provided herein wherein the composition is suitable for local administration. In some embodiments, local administration comprises intratumoral injection, peritumoral injection, juxtatumoral injection, intralesional injection and/or injection into a tumor draining lymph node, or essentially any tumor-targeted injection where the antitumor agent is expected to leak into primary lymph nodes adjacent to targeted solid tumor.

Pharmaceutically acceptable carriers that can be used in compositions or formulations provided herein include any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active ingredient, i.e., the antibodies, fusion proteins or cells, can be coated in a material to protect the active ingredient from the action of acids and other natural conditions that can inactivate the active ingredient.

### Methods and Uses

Provided herein are also uses of the antibodies or antigen-binding fragments, the fusion proteins, and the immune effector cells disclosed herein.

The anti-CD40 antibodies or antigen-binding fragments and pharmaceutical compositions comprising the anti-CD40 antibodies or antigen-binding fragments described herein have numerous in vitro and in vivo utilities involving, for example, enhancement of immune response. For example, anti-CD40 antibodies or fusion proteins disclosed herein can be administered to cells in culture, in vitro or ex vivo, or to human subjects, e.g., in vivo, to enhance immunity in a variety of diseases.

The enhancement of immune response can include the activation of immune cells. For example, activation of T cells, B cells, and dendritic cells (DC cells). The activation of immune cells can comprise but not limited to the upregulation of immune cell activation markers, the increased cytokine secretion by immune cells, the proliferation of immune cells and/or the enhanced immune cell cytotoxicity. The method for detecting the enhancement of immune response is known in the art. The enhancement of the immune response can be comprehensively assessed through quantitative analysis of the expression of activation markers, the concentration of cytokines, the proliferation index of immune cells, and the results of functional assays. The degree of activation can be quantified by comparing the differences between the experimental group and the control group, thereby determining the effectiveness of a specific substance or intervention in enhancing the immune response.

Provided herein are methods of modifying an immune response in a subject comprising administering to the subject the antibody or antigen-binding fragment, fusion protein, or cells described herein such that the immune response in the subject is enhanced, stimulated or up-regulated.

Given the ability of anti-CD40 antibodies or antigen-binding fragments described herein to enhance co-stimulation of T cell responses, e.g., antigen-specific T cell responses, provided herein are in vitro and in vivo methods of using the antibody or antigen-binding fragment, fusion protein, or cell described herein to stimulate, enhance or upregulate antigen-specific T cell responses, e.g., anti-tumor T cell responses. CD4⁺ and CD8⁺ T cell responses can be enhanced using anti-CD40 antibodies or antigen-binding fragments. The T cells can be CD4+ T cells, CD8+ T cells, T helper (Tₕ) cells and T cytotoxic (T_{c}) cells.

Further encompassed are methods of enhancing an immune response (e.g., an antigen-specific T cell response) in a subject comprising administering an antibody or antigen-binding fragment, fusion protein, or cell described herein to the subject such that an immune response (e.g., an antigen-specific T cell response) in the subject is enhanced. In a preferred embodiment, the subject is a tumor-bearing subject and an immune response against the tumor is enhanced. A tumor can be a solid tumor or a liquid tumor, e.g., a hematological malignancy. In some embodiments, a tumor is an immunogenic tumor. In some embodiments, a tumor is non-immunogenic. In some embodiments, a tumor is PD-L1 positive. In some embodiments a tumor is PD-L1 negative. A subject can also be a virus-bearing subject and an immune response against the virus is enhanced.

Further provided are methods for inhibiting growth of tumor cells in a subject comprising administering to the subject an antibody or antigen-binding fragment, fusion protein, or cell described herein such that growth of the tumor is inhibited in the subject. Also provided are methods of treating chronic viral infection in a subject comprising administering to the subject an antibody or antigen-binding fragment, fusion protein, or cells described herein such that the chronic viral infection is treated in the subject.

Provided herein are methods for treating a subject having cancer, comprising administering to the subject an antibody or antigen-binding fragment, fusion protein, or cell described herein, such that the subject is treated, e.g., such that growth of cancerous tumors is inhibited or reduced and/or that the tumors regress. An anti-CD40 antibody or antigen-binding fragment, fusion protein, or cell can be used alone to inhibit the growth of cancerous tumors. Alternatively, an anti-CD40 antibody or antigen-binding fragment, fusion protein, or cell can be used in conjunction with another agent, e.g., other immunogenic agents, standard cancer treatments, or other antibodies, as described below. Combination with an inhibitor of PD-1, such as an anti-PD-l or anti-PD-L1 antibody, is also provided. See, e.g., Ellmark et al. (2015) Oncolmmunology 4:7 elOH484. The anti-CD40 antibodies or antigen-binding fragments thereof, the fusion proteins and/or the cells disclosed herein can also be combined with standard cancer treatments (e.g., surgery, radiation, and chemotherapy). The anti-CD40 antibodies or antigen-binding fragments thereof described herein can be co-administered with one or other more therapeutic agents, e.g., a cytotoxic agent, a radiotoxic agent or an immunosuppressive agent. The antibody or antigen-binding fragments thereof can be linked to the agent (as an immuno-complex) or can be administered separate from the agent. In the latter case (separate administration), the antibody or antigen-binding fragments thereof can be administered before, after or concurrently with the agent or can be co-administered with other known therapies, e.g., an anti-cancer therapy, e.g., radiation.

Accordingly, provided herein are methods of treating cancer, e.g., by inhibiting growth of tumor cells, in a subject, comprising administering to the subject a therapeutically effective amount of an anti-CD40 antibody or antigen-binding fragment described herein, e.g., 406, 409, 4011, 4012, 4021, 4025, 4034, 4037, 4044, 4045 or 4052. Provided herein are methods of treating cancer comprising administering to the subject a therapeutically effective amount of a fusion protein described herein. Provided herein are methods of treating cancer comprising administering to the subject a therapeutically effective amount of a cell described herein.

Cancers whose growth can be inhibited using the antibodies disclosed herein include cancers typically responsive to immunotherapy. The cancer described in the present application may include cancer expressing the aforementioned tumor antigen. Non-limiting examples of cancers for treatment include squamous cell carcinoma, small-cell lung cancer, non-small cell lung cancer, squamous non-small cell lung cancer (NSCLC), non NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (Ml), myeloblastic leukemia (M2; with cell maturation), promyelocytic leukemia (M3 or M3 variant [M3 V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitf s lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-l antibody), and recurrent cancers.

In some embodiments, the antibodies or antigen-binding fragments described herein can be used to treat an infectious disease in a subject in need thereof. Examples of pathogens for which this therapeutic approach can be particularly useful, include, but are not limited to COVID-19, HIV, Hepatitis (A, B, & C), Influenza, Herpes, Giardia, Malaria, Leishmania, Staphylococcus aureus, Pseudomonas aeruginosa. CD40 agonism is particularly useful against established infections by agents such as HIV that present altered antigens over the course of the infections. These novel epitopes are recognized as foreign at the time of anti-human CD40 antibody administration, thus provoking a strong T cell response.

Provided herein are the uses of the antibody or antigen-binding fragment, the fusion protein, or the cell described herein to treat a cancer and/or a tumor. Provided herein are the uses of the antibody or antigen-binding fragment, the fusion protein, or the cell described herein to treat an infectious disease.

Provided herein are the antibody or antigen-binding fragment, the fusion protein, or the cell described herein for use in the treatment of a cancer and/or a tumor. Provided herein are the antibody or antigen-binding fragment, the fusion protein, or the cell described herein for use in the treatment of an infectious disease.

Provided herein are uses of the antibody or antigen-binding fragment, the fusion protein, or the cell described herein for the manufacture of a pharmaceutical composition. In some embodiments, the pharmaceutical composition is for the treatment of a cancer and/or a tumor. In some embodiments, the pharmaceutical composition is for the treatment of an infectious disease.

Also encompassed are methods for detecting the presence of CD40 (e.g., human CD40) in a sample, or measuring the amount of CD40, comprising contacting the sample, and a control sample, with an antibody or antigen-binding fragment, fusion protein, or cell described herein, under conditions that allow for formation of a complex between the antigen-binding fragment, fusion protein, or cell and CD40. The formation of a complex is then detected, wherein a difference complex formation between the sample compared to the control sample is indicative of the presence of the CD40 molecules in the sample. Moreover, the anti-CD40 antibodies and antigen-binding fragments described herein can be used to purify CD40 via immunoaffinity purification.

The anti-CD40 antibodies or antigen-binding fragments thereof provided herein can be used to enhance antigen-specific immune responses by co-administration of an anti-CD40 antibody with an antigen of interest, e.g., a vaccine. The anti-CD40 antibodies or antigen-binding fragments thereof provided herein can be used as vaccine adjuvants. Accordingly, provided herein are methods of enhancing an immune response to an antigen in a subject, comprising administering to the subject: (i) the antigen; and (ii) an anti-CD40 antibody, or antigen-binding fragment thereof, such that an immune response to the antigen in the subject is enhanced. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above, or antigens from the viruses, bacteria or other pathogens described above.

### Methods of Genetic Engineering

With respect to generating cells recombinantly expressing a fusion protein disclosed herein, one or more polynucleotides encoding the fusion protein is introduced into the target cell using a suitable expression vector. The target immune effector cells (e.g., T cells) are transferred with one or more polynucleotides encoding a fusion protein, or a CAR/TCR/BiTE and a fusion protein. The CAR/TCR/BiTE and fusion protein encoding polynucleotides can be on separate vectors or on the same vector, as desired. For example, a polynucleotide encoding a CAR or a fusion protein disclosed herein can be cloned into a suitable vector, such as a viral vector, and introduced into the target cell using well known molecular biology techniques (see Ausubel et al., Current Protocols in Molecular Biology, John Wiley and Sons, Baltimore, MD (1999)). Any vector suitable for expression in a cell, particularly a human cell, can be used. The vectors contain suitable expression elements such as promoters that provide for expression of the encoded nucleic acids in the target cell. In the case of a retroviral vector, cells can optionally be activated to increase transduction efficiency (see Parente-Pereira et al., J. Biol. Methods 1(2) e7 (doi 10.14440/jbm.2014.30) (2014); Movassagh et al., Hum. Gene Ther. 11:1189-1200 (2000); Rettig et al., Mol. Ther. 8:29-41 (2003); Agarwal et al., J. Virol. 72:3720-3728 (1998); Polloketal., Hum. Gene Ther. 10:2221-2236 (1998); Quinn et al., Hum. Gene Ther. 9:1457-1467 (1998); see also commercially available methods such as Dynabeads™ human T cell activator products, Thermo Fisher Scientific, Waltham, MA).

In one embodiment, the vector is a retroviral vector, for example, a gamma retroviral or lentiviral vector, which is employed for the introduction of a fusion protein and/or a CAR, TCR, or BiTE into the target cell. For genetic modification of the cells to express a fusion protein and/or a CAR, TCR, or BiTE, a retroviral vector can be employed for transduction. However, it is understood that any suitable viral vector or non-viral delivery system can be used. Combinations of a retroviral vector and an appropriate packaging line are also suitable, where the capsid proteins will be functional for infecting human cells. Various amphotropic virus-producing cell lines are known, including, but not limited to, PA12 (Miller et al.,Mol. Cell. Biol. 5:431-437 (1985)); PA317 (Miller et al.,Mol. Cell. Biol. 6:2895-2902(1986)); and CRIP (Danos et al., Proc. Natl. Acad. Sci. USA 85:6460-6464 (1988)). Non-amphotropic particles are suitable too, for example, particles pseudotyped with VSVG, RD114 or GALV envelope and any other known in the art (Relander et al., Mol. Therap. 11:452-459 (2005)). Possible methods of transduction also include direct co-culture of the cells with producer cells (for example, Bregni et al., Blood 80:1418-1422 (1992)), or culturing with viral supernatant alone or concentrated vector stocks with or without appropriate growth factors and polycations (see, for example, Xu et al., Exp. Hemat. 22:223-230 (1994); Hughes, et al. J. Clin. Invest. 89:1817-1824 (1992)).

Other viral vectors that can be used include, for example, adenoviral, lentiviral, and adeno-associated viral vectors, vaccinia virus, a bovine papilloma virus derived vector, or a herpes virus, such as Epstein-Barr Virus (see, for example, Miller, Hum. Gene Ther. 1(1):5-14 (1990); Friedman, Science 244:1275-1281 (1989); Eglitis et al., BioTechniques 6:608-614 (1988); Tolstoshev et al., Current Opin. Biotechnol. 1:55-61 (1990); Sharp, Lancet 337:1277-1278 (1991); Cornetta et al., Prog. Nucleic Acid Res. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med. 323:370 (1990); Anderson et al., U.S. Pat. No. 5,399,346). Generally, the chosen vector exhibits high efficiency of infection and stable integration and expression (see, for example, Cayouette et al., Human Gene Therapy 8:423-430 (1997); Kido et al., Current Eye Research 15:833-844 (1996); Bloomer et al., J Virol. 71:6641-6649 (1997); Naldini et al., Science 272:263-267 (1996); and Miyoshi et al., Proc. Natl. Acad. Sci. U.S.A. 94:10319-10323 (1997)).

Particularly useful vectors for expressing a fusion protein disclosed herein and/or CAR/TCR/BiTE include vectors that have been used in human gene therapy. In one non-limiting embodiment, a vector is a retroviral vector. The use of retroviral vectors for expression in T cells or other immune effector cells, including engineered T cells, has been described (see Scholler et al., Sci. Transl. Med. 4:132-153 (2012; Parente-Pereira et al., J. Biol. Methods 1(2):e7 (1-9)(2014); Lamers et al., Blood 117(1):72-82 (2011); Reviere et al., Proc. Natl. Acad. Sci. USA 92:6733-6737 (1995)). In one embodiment, the vector is an SGF retroviral vector such as an SGF γ-retroviral vector, which is Moloney murine leukemia-based retroviral vector. SGF vectors have been described previously (see, for example, Wang et al., Gene Therapy 15:1454-1459 (2008)).

The vectors used herein employ suitable promoters for expression in a particular host cell. The promoter can be an inducible promoter or a constitutive promoter. In some embodiments, the promoter of an expression vector provides expression in a stem cell, such as a hematopoietic stem cell. In some embodiments, the promoter of an expression vector provides expression in an immune effector cell, such as a T cell. Non-viral vectors can be used as well, so long as the vector contains suitable expression elements for expression in the target cell. Some vectors, such as retroviral vectors, can integrate into the host genome.

In some embodiments, provided herein are methods of genetically engineering an immune effector cell by transferring a polynucleotide provided herein into the cell using a non-viral delivery system. For example, physical methods for introducing a polynucleotide into a host cell include calcium phosphate precipitation, lipofection, particle bombardment, microinjection, electroporation, and the like. In some embodiments, RNA electroporation can be used (Van Driessche et al. Folia histochemica et cytobiologica 43:4 213-216 (2005)). In some embodiments, DNA transfection and transposon can be used. In some embodiments, the Sleeping Beauty system or PiggyBac system is used (e.g., Ivics et al., Cell, 91 (4): 501-510 (1997); Cadiñanos et al. (2007) Nucleic Acids Research. 35 (12): e87). Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, provided herein are methods of genetically engineering an immune effector cell by transferring a polynucleotide provided herein into the cell using gene-editing. If desired, targeted integration can be implemented using technologies such as a nuclease, transcription activator-like effector nucleases (TALENs), Zinc-finger nucleases (ZFNs), clustered regularly interspaced short palindromic repeats (CRISPRs), homologous recombination, non-homologous end joining, microhomology-mediated end joining, homology-mediated end joining and the like (Gersbach et al., Nucl. Acids Res. 39:7868-7878 (2011); Vasileva, et al. Cell Death Dis. 6:e1831. (Jul. 23 2015); Sontheimer, Hum. Gene Ther. 26(7):413-424 (2015); Yao et al. Cell Research volume 27, 801-814(2017)).

Immune effector cells provided herein can be obtained from a subject. Sources for the immune effector cells provided herein include, but are not limited to, peripheral blood, umbilical cord blood, bone marrow, or other sources of hematopoietic cells. Immune effector cells (e.g., T cells) can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments, cell lines available in the art can be used. Immune effector cells provided herein can be isolated by methods well known in the art, including commercially available isolation methods (see, for example, Rowland-Jones et al., LYMPHOCYTES: A PRACTICAL APPROACH, Oxford University Press, New York (1999)). Various methods for isolating immune effector cells have been described previously, and can be used, including but not limited to, using peripheral donor lymphocytes (Sadelain et al., Nat. Rev. Cancer 3:35-45 (2003); Morgan et al., Science 314: 126-129 (2006), and using selectively in v/Yro-expanded antigen-specific peripheral blood leukocytes employing artificial antigen-presenting cells (AAPCs) or dendritic cells (Dupont et al., Cancer Res. 65:5417-5427 (2005); Papanicolaou et al., Blood 102:2498-2505 (2003)).

The immune effector cells can be autologous or non-autologous to the subject to which they are administered in the methods of treatment disclosed herein. Autologous cells are isolated from the subject to which the engineered cells are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine an appropriate level of compatibility, as is well known in the art. The cells can optionally be cryopreserved after isolation and/or genetic engineering, and/or expansion of genetically engineered cells (see Kaiser et al., supra, 2015)). Methods for cyropreserving cells are well known in the art (see, for example, Freshney, CULTURE OF ANIMAL CELLS: A MANUAL OF BASIC TECHNIQUES, 4^{th} ed., Wiley-Liss, New York (2000); Harrison and Rae, GENERAL TECHNIQUES OF CELL CULTURE, Cambridge University Press (1997)).

In some embodiments, isolated immune effector cells are genetically engineered ex vivo for recombinant expression of a fusion protein. In some embodiments, isolated immune effector cells are genetically engineered ex vivo for recombinant expression of a fusion protein and a CAR/TCR/BiTE. In some embodiments, immune effector cells provided herein are obtained by in vitro sensitization, wherein the sensitization can occur before or after the immune effector cells are genetically engineered to recombinantly express the fusion protein disclosed herein. In an embodiment where the sensitized immune effector cells, such T cells, are isolated from in vivo sources, it will be self-evident that genetic engineering occurs of the already-sensitized immune effector cells.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

In this study, we selected some scFvs against CD40 from fully human antibody phage display library, then the candidate scFv sequences were constructed into the expression vectors, and IVT was performed to obtain anti-CD40 scFv mRNA. Then the mRNA was electrotransfected into human T cells to verify the binding ability to CD40 protein. Then the candidate sequences were further constructed into LACO-CAR vectors, and lentiviral CAR-T cells were prepared to further verify the activation effects of different CD40 molecules on DC cells at the cellular level and mouse tumor models, as well as to enhance CAR-Naïve killing effect of.

### Example 1 Construction MOLM14-CD40 Stable Cell Line

The full-length CD40 gene sequence was cloned into the lentiviral vector pUTCK, and after lentiviral packaging, MOLM14 cells were infected. On the third day after infection, the cells were stained with anti-CD40 flow cytometry antibodies. MOLM14 cells positive for CD40 antibody staining were sorted using flow cytometry and continued to be expanded and cultured. As shown in FIG. 1, the sorted MOLM14-CD40 cell line expresses high levels of CD40 protein.

### Example 2 Anti-CD40 Antibodies Screening with Phage Display Library

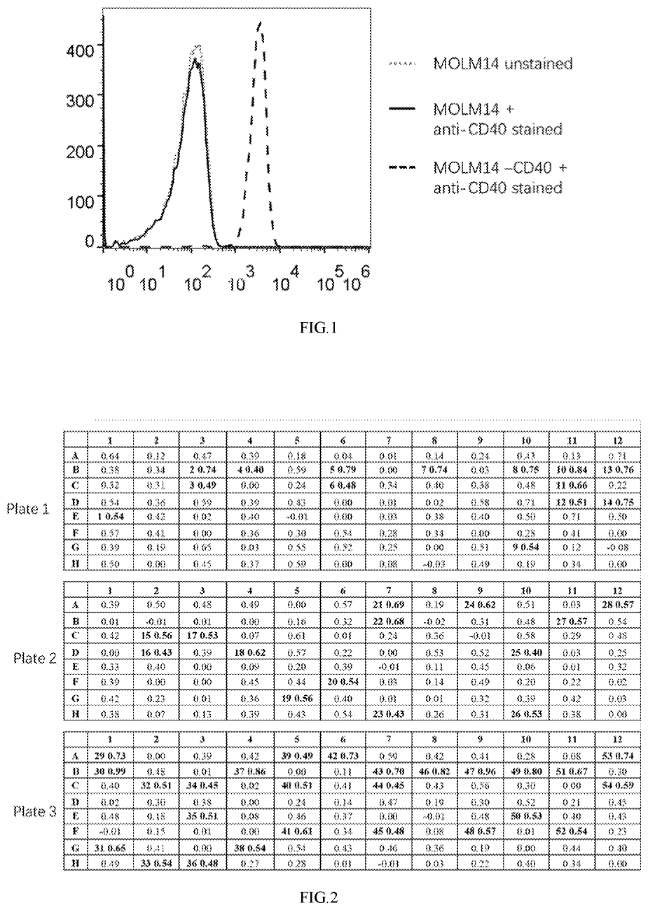

According to the initial OD value of about 0.1, an appropriate amount of original phage library liquid was inoculated into a 500 ml Erlenmeyer flask with 2×YT medium, and added 100 g/mL ampicillin and 2% glucose. The flask was shaken for about 3 hours at 250 rpm, 37° C., until the OD600 value was about 0.5. 3×10¹² M13K07 helper phage was added and placed the culture at 37° C. for 30 minutes. After 30 minutes, the culture was transferred to a 50 ml centrifuge tube, centrifuged at 2400 g, 12° C. for 30 minutes, and the supernatant was discarded to remove the inhibitory effect of glucose on antibody expression. Resuspended the pellet to 600 ml 2×YT, and add ampicillin, 60 μg/mL kanamycin, and 100 μM IPTG at a final concentration of 100 μg/mL). Shaked culture overnight at 30° C., 200 rpm. The next day, collected the bacterial liquid, centrifuge at 2400 g, 12° C. for 30 minutes. After centrifugation, transferred 36 ml of the supernatant to a new 50 ml centrifuge tube, then added 9 ml of PEG/NaCl, mixed thoroughly, and placed on ice for 1-2 hours. After 1-2 hours, centrifuged the mixture at 4000 rpm and 4° C. for 30 minutes. After centrifugation, gently discarded the supernatant, resuspended the phage pellet in 3 ml of 1×PBS/5% FBS solution, and stored it at 4 degrees for later use. Mixed the purified phage library and MOLM14-CD40 (resuspended in 5% FBS/PBS) evenly, and spined at 10 rpm on a mixer in a refrigerator at 4 degrees. After 1.5 hours, washed the cells with 5% FBS/PBS and removed as much supernatant as possible. The phage was eluted with 500 μl of 0.1 M, pH2.2 glycine and placed at room temperature for 10 minutes. After centrifugation, transferred the supernatant to a new tube and added 50 ul pH9.5 Tris-HCL to neutralize the pH to neutral. Added the eluted phage to 1E7/2.5 ml MOLM14 (resuspend in 5% FBS/PBS), mixed well, rotated at 10 rpm, and combined for about 0.5 hours. Centrifuged and repeated step 11 two more times. After the last centrifugation, transferred the supernatant to a new 15 ml tube and used it to infect TG1. Repeated the panning operation twice more, and the clones finally obtained were tested by whole-cell phage ELISA (the results are shown in FIG. 2. The positive clones were amplified by PCR to obtain the scFv sequence (the bold in FIG. 2 is the PCR identification clone).

### Example 3 In Vitro Transcription (IVT) Preparation and Purification of mRNA

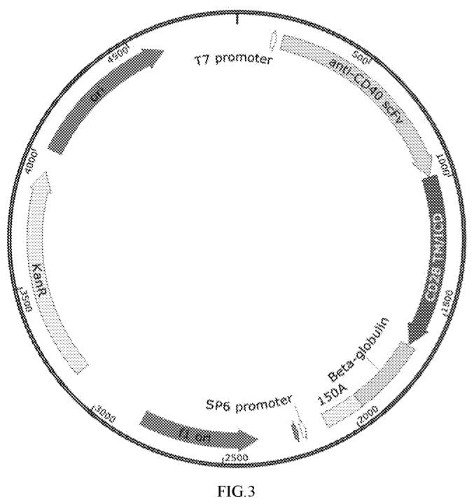

1. The anti-CD40 subsequent clone sequences obtained by sequencing were cloned into the pDA.CD28 plasmid vector (as shown in FIG. 3). 2. The plasmids encoding pDA.CD40.CD28 were linearized with BspQ1 restriction enzyme. 3. The linearized plasmids were purified using a PCR cleanup kit (Qiagen) and eluted with Rnase-free water. 4. DNA concentration was measured by Nanodrop and checked by running an agarose DNA gel. 5. Performed the in vitro transcription (IVT) according to the manufacturer's standard operating procedure (Thermofisher, Cat. No.: AMB1345). Briefly, added 1 μg of template DNA, ARCA/NTP buffer, 1OX reaction buffer, T7 enzyme, and Rnase-free HO as a volume of 20 μl to a 0.2 mL PCR tube and incubated at 37° C. for 4 h. 6. After 4 hours, added 2 μl of Dnase I to each reaction and incubated at 37° C. for 15 minutes. 7. Purified the IVT mRNA using Rnasy kit (Qiagen). 8. Purified mRNA concentration was measured by Nanodrop and checked by PAGE gel.

### Example 4 Tumor Cells and T Cells Culture

Tumor cell lines were cultured in RPMI-1640 or DMEM medium containing 10% fetal bovine serum and 1% double-resistance, and were passaged once every 2-3 days. Primary CD3+ T cells isolated and purified from PBMC were activated with anti-CD3/CD28 Dynabeads (Thermofisher, catalog number: 402031) and cultured in R10 medium (RPMI-1640 basic medium plus 10% fetal bovine serum, 1% double-resistance, 1% HEPES, 1% sodium pyruvate, 1% glutamax and 1% non-essential amino acids (NEAA). On the 4^{th} day, removed the magnetic beads from the T cells and continued to culture. After culturing for 13 days, harvested the T cells used for subsequent electroporation of mRNA to prepare CAR-T cells.

### Example 5 T Cell mRNA Electroporation

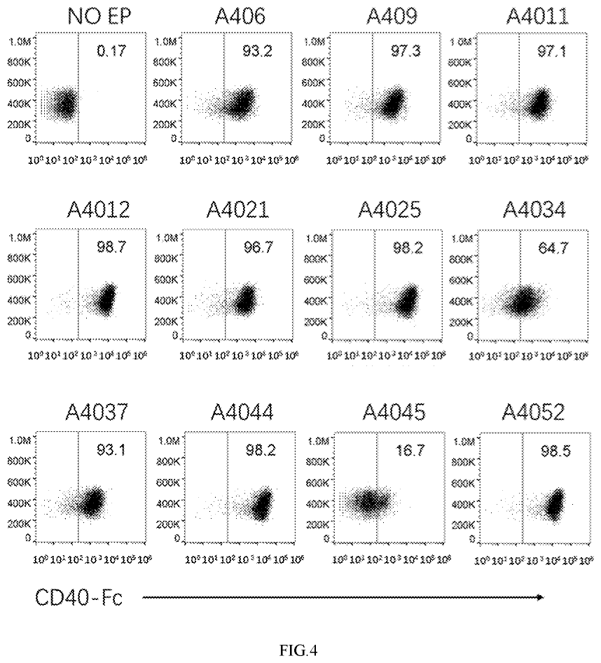

Collected the T cells according to the Example 4 after activation for 13 days and washed them three times with Opti-MEM medium. Suspension cultured the cells in Opti-MEM medium and adjusted the cell concentration to 5×10e7/ml. Mixed the required volume of mRNA and 100 μl T cells gently, and electroporated immediately. The parameters were set on the BTX ECM 830 machine: 500V voltage, 0.5 milliseconds. Added the mRNA and cell mixture into the BTX electroporation cup and tapped gently to avoid air bubbles. Performed electroporation, then transferred the electroporated cells to 1 ml of preheated culture medium, mixed evenly, and then placed it in a 37-degree incubator to continue culturing. The protein expression level was detected on the second day after electroporation. The results are shown in FIG. 4. The staining results show that among the 11 anti-CD40 scFv antibodies, A406, A409, A4011, A4012, A4021, A4025, A4037, and A4044 And A4052 anti-CD40 scFv has a strong ability to bind to CD40-Fc protein. A4034 and A4045 anti-CD40 scFv have relatively weak ability to bind to CD40-Fc protein.

### Example 6 LACOs Comprises Different First Binding Domain

The LACO/MSLN CAR sequences of 9 candidate anti-CD40 scFv were cloned into the pUTCK vector (as shown in FIG. 5). The MSLN CAR in Examples 6-9 comprises the amino sequence as set forth in SEQ ID NO. 122. The LACO in this example comprises anti-CD40 scFv as the first domain and CD28 TM/ICD as the second domain, wherein the first domain is different. The amino acid sequences of the LACO in this example are shown as set forth in SEQ ID NO: 155-160, 162-163, and 165, respectively.

And carried out plasmid extraction, lentivirus packaging, concentration and titer determination, and froze it in a −80° C. refrigerator for later use. Preparation of CAR-T cells. On day 1, T cells were activated with anti-CD3/CD28 Dynabeads, and the cell density was adjusted to 1e6/ml. On day 2, added the lentivirus to T cells according to MOI=3 and proceeded with infection. On day 5, removed the anti-CD3/CD28 Dynabeads, and performed cell counting, passage and supplementation of culture medium. On day 9, 3e5 T cells were stained with MSLN-Fc recombinant protein and anti-human IgG Fc antibody, and performed flow cytometry analysis to detect the CAR positivity rate. Or stained with CD40-Fc recombinant protein and anti-human IgG Fc antibody staining, flow cytometry analysis, and detect the expression of LACO-STIM. Continue to culture the CAR-T cells until about 13 days, harvested the CAR-T cells for functional experiments, or froze them for later use.

### Example 7 Activation Effect of LACO/MSLN CAR-T Cells on DC Cells

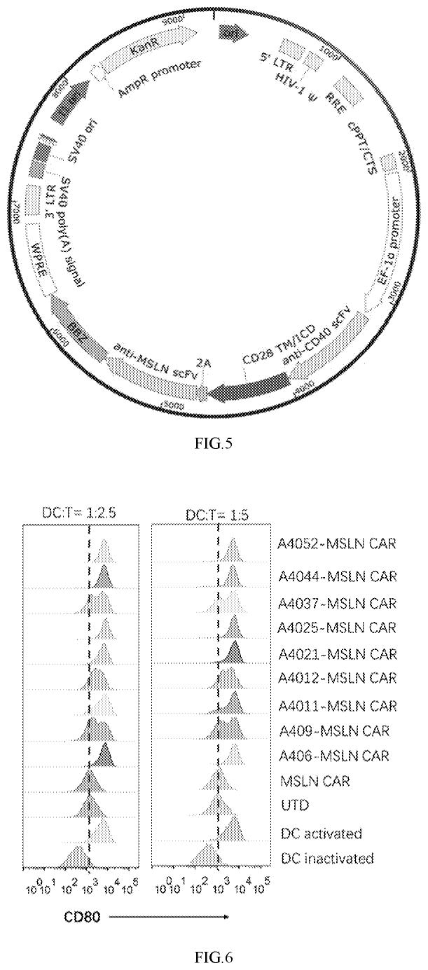

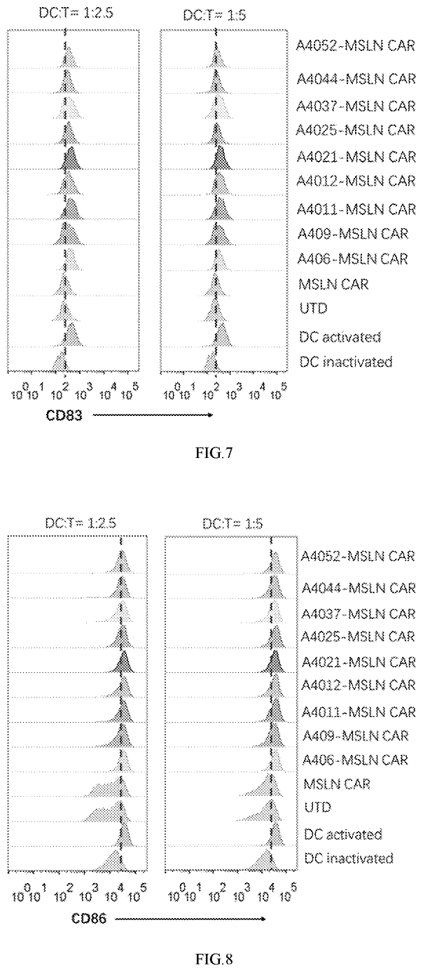

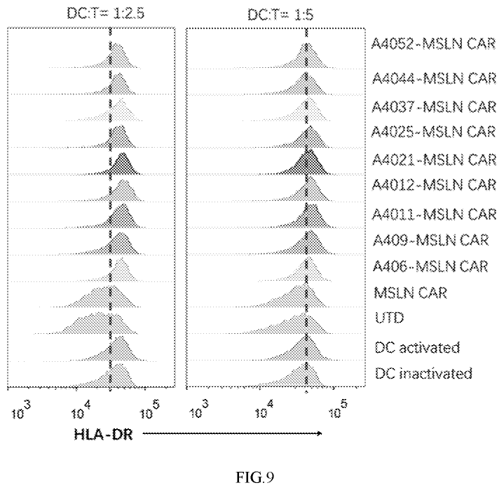

Added 1×10⁵ DC cells to each well of a low-adsorption 24-well plate, then added 5×10⁵ CAR-T cells, and incubated for 48 hours. After 48 hours, gently pipetted to detach the adherent DC cells from the 96-well wall. DC cells were collected for flow cytometric staining with CD11b, CD80, CD83, CD86, HLA-DR and other antibodies. After staining, the expression of various markers was detected by flow cytometry analysis (as shown in FIG. 6-9). It can be seen that MSLN CAR-T cells and LACO/MSLN CAR-T cells activate DC cells, and the activation effect is enhanced to varying degrees when co-expressed with LACO.

### Example 8 In Vitro Efficacy Evaluation of LACO/MSLN CAR-T Cells

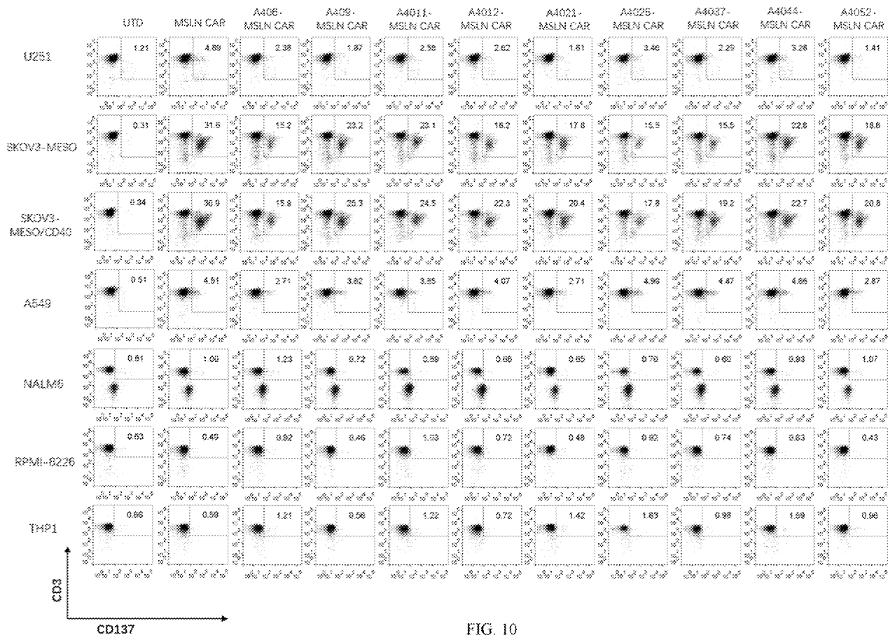

Inoculated various tumor cells into a 96-well plate at 1×10⁵ cells/100 μl per well. Diluted various CAR-T cells to appropriate cell density, then add 1×10⁵ cells/100 μl per well to the 96-well plate of the above-mentioned tumor cell lines, and incubated with tumor cells. 37° C. culture medium was added into the 96-well plate and incubated for 24 hours. Then collected T cells for flow cytometry staining, and stain antibodies CD3 and CD137 for flow cytometry analysis (as shown in FIG. 10).

### Example 9 In Vivo Efficacy Evaluation of LACO/MSLN CAR-T Cells

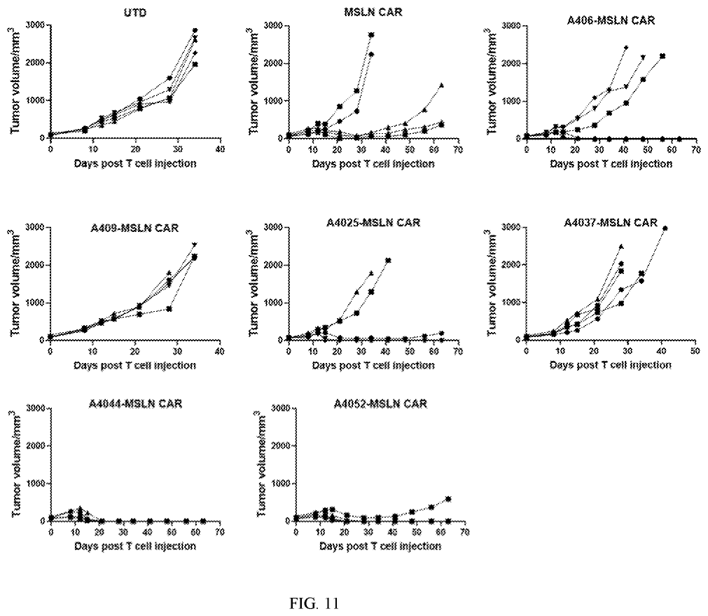

M-NSG mice were inoculated with SKOV3-MSLN/CD40 cells as each mouse with 5×10⁶ cells. About 20 days after inoculation, when the tumor volume had grown to an average of 80-100 mm³, CAR-T cells were reinfused through tail vein injection, and each mouse was reinfused with 1×10⁶ cells. Measured tumor volume and weight once a week and performed statistical analysis (as shown in FIG. 11).

### Example 10 LACOs Comprises Different Second Binding Domain

Collect A549 tumor cells and wash them three times with Opti-MEM medium. Resuspend the cells in Opti-MEM medium and adjust the cell concentration to 5×10e7/ml. Mix the required volume of mRNA and 100 μl of tumor cells gently, and electroporate immediately. Set parameters on the BTX ECM 830 machine: 360V voltage, 1 millisecond pulse duration. Add the mRNA/cell mixture into the BTX electroporation cup and tap gently to avoid air bubbles. Perform electroporation, then transfer the electroporated cells to 1 ml of preheated culture medium, mix evenly, and then place them in a 37° C. incubator for further culturing.

Clone MSLN CAR sequences and 16 MSLN CAR/LACO sequences into the pUTCK vector respectively (the exemplary schematic diagram of co-expression of MSLN CAR and LACO can be seen in FIG. 47). The 16 MSLN CAR/LACO sequences comprise same CAR sequence but different LACO, wherein the second binding domain is different. The information of MSLN CAR and MSLN CAR/LACOs is shown in the Table 8.

TABLE 8
MSLN CAR and MSLN CAR/LACO

| CAR/CAR-LACO | LACO | Amino Acid Sequence |
|---|---|---|
| MSLN CAR | — | SEQ ID NO: 122 |
| MSLN-A4025-9.3h11 | A4025-9.3h11 | SEQ ID NO: 194 |
| MSLN-A4025-9.3h12 | A4025-9.3h12 | SEQ ID NO: 246 |
| MSLN-A4025-9.3h22 | A4025-9.3h22 | SEQ ID NO: 247 |
| MSLN-A4025-9.3h23 | A4025-9.3h22 | SEQ ID NO: 248 |
| MSLN-A4025- 9.3h31 | A4025- 9.3h31 | SEQ ID NO: 249 |
| MSLN-A4025-9.3h32 | A4025-9.3h32 | SEQ ID NO: 250 |
| MSLN-A4025-9.3h33 | A4025-9.3h33 | SEQ ID NO: 251 |
| MSLN-A4025-9.3h42 | A4025-9.3h42 | SEQ ID NO: 252 |
| MSLN-A4025-9.3h43 | A4025-9.3h43 | SEQ ID NO: 253 |
| MSLN-A4025-9.3h52 | A4025-9.3h52 | SEQ ID NO: 254 |
| MSLN-A4025-9.3 | A4025-9.3 | SEQ ID NO: 255 |
| MSLN-A4025-C65 | A4025-C65 | SEQ ID NO: 256 |
| MSLN-A4025-C58 | A4025-C58 | SEQ ID NO: 257 |
| MSLN-A4025-28.3 | A4025-28.3 | SEQ ID NO: 258 |
| MSLN-A4025-h28.3 | A4025-h28.3 | SEQ ID NO: 259 |
| MSLN-A4025-1412 | A4025-1412 | SEQ ID NO: 260 |

Then, perform plasmid extraction, lentivirus packaging, ultracentrifugation, and titer determination. Finally, freeze them in a −80° C. refrigerator for later use. Preparation of CAR-T cells: On day 1, activate T cells with anti-CD3/CD28 Dynabeads, and adjust the cell density to 1e6 cells/ml. On day 2, add lentivirus to T cells at an MOI of 3 and culture for another two days. On day 5, remove the anti-CD3/CD28 Dynabeads, perform cell counting, passage and rehydration. Continue to culture the CAR-T cells until about day 13, harvest them, and use them directly for functional experiments, or freeze them for later use.

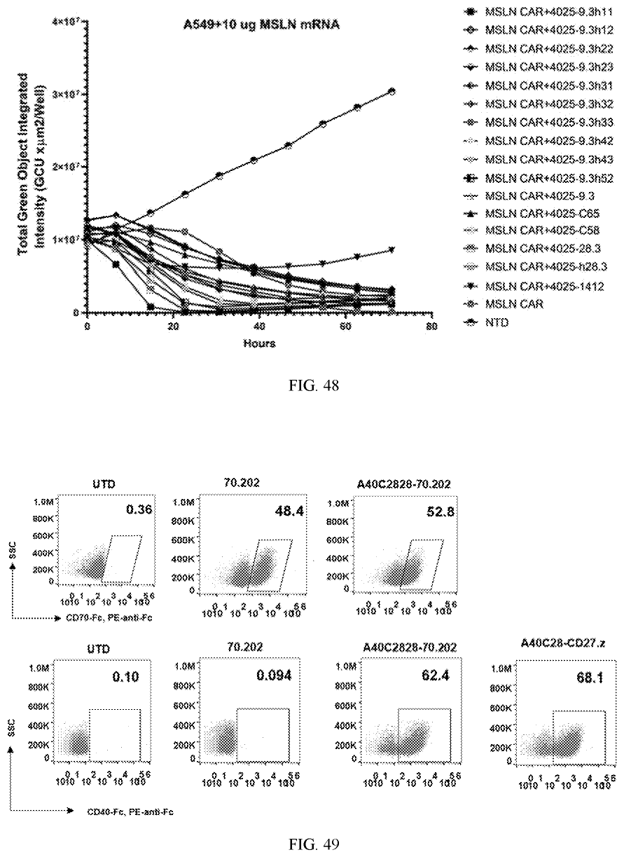

Twelve hours prior to co-culture for cytotoxicity experiments with CAR-T cells, seed tumor cells into a flat-bottom 96-well plate at a density of 3,000-10,000 cells/100 μl per well. After 8-12 hours, allow the cells to fully adhere to the well surface. Dilute various anti-MSLN CAR-T cells to appropriate cell densities and co-incubate them with tumor cells at E:T=3:1). Place the 96-well plate into the InCucyte S3 machine and set the scanning parameters. After 3 days of scanning, analyze the total green fluorescence cumulative intensity (GCU×μm²/well) to calculate tumor cell killing efficiency. The results are shown in FIG. 48. Compared with NTD cells, MSLN CAR-T cells, and 16 types of MSLN CAR/LACO CAR-T cells, have high killing efficiency against A549 cells electroporated with MSLN mRNA.

### Example 11 Preparation of Lentiviral Anti-HER2 CAR-T Cells

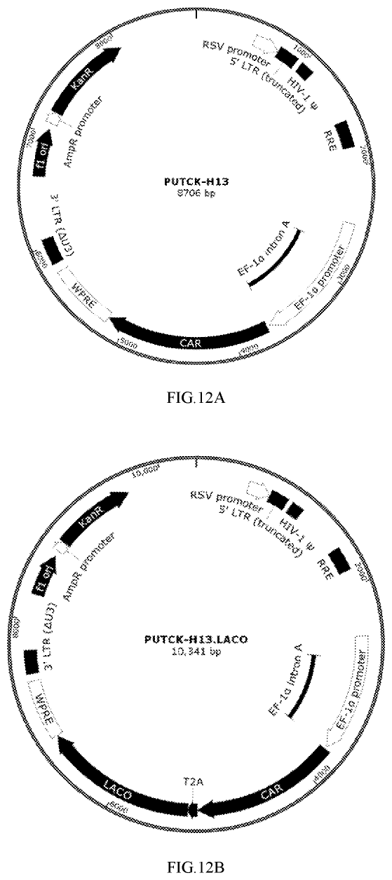

H13 HER2 CAR sequence and 2 HER2 CAR/LACO sequences were cloned into the pUTCK vector respectively (as shown in FIG. 12A-12B). The sequences of CAR and different LACOs are shown in SEQ ID NO: 187-191, wherein the sequence of SEQ ID NO: 188 is H13 HER2 CAR and the membrane LACO of A4025-CD28, the sequence of SEQ ID NO: 189 is H13 HER2 CAR and the membrane LACO of A40517-CD28, the sequence of SEQ ID NO: 190 is H13 HER2 CAR and the soluble LACO (sLACO) of A4025-9.3 h11, the sequence of SEQ ID NO: 191 is H13 HER2 CAR and the sLACO of A40517-9.3 h11. A4025 and A40517 are different anti-CD40 antibodies, 9.3h11 is an anti-CD28 antibody.

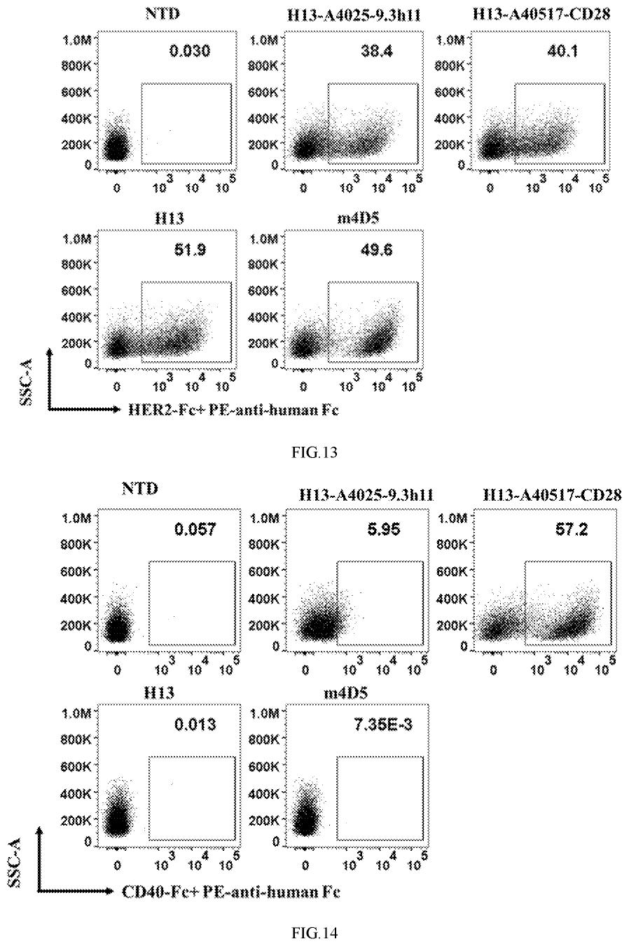

Then, perform plasmid extraction, lentivirus packaging, ultracentrifugation, and titer determination. Finally, freeze them in a −80° C. refrigerator for later use. Preparation of CAR-T cells: On day 0, activate T cells with anti-CD3/CD28 Dynabeads, and adjust the cell density to 1e6 cells/ml. On day 1, add lentivirus to T cells at an MOI of 3 and culture for another two days. On day 5, remove the anti-CD3/CD28 Dynabeads, perform cell counting, passage and rehydration. On day 9, take 3e5 T cells from each group, stain them with HER2-Fc/anti-human IgG Fc and CD40-Fc/anti-human IgG Fc antibodies respectively, conduct flow cytometry analysis to detect the CAR positivity rate, and assess the expression of LACO (as shown in FIG. 13).

Continue to culture the CAR-T cells until about day 13, harvest them, and use them directly for functional experiments, or freeze them for later use.

### Example 12 In Vitro Cytotoxicity Testing of Anti-HER2 CAR-T Cell Preparation

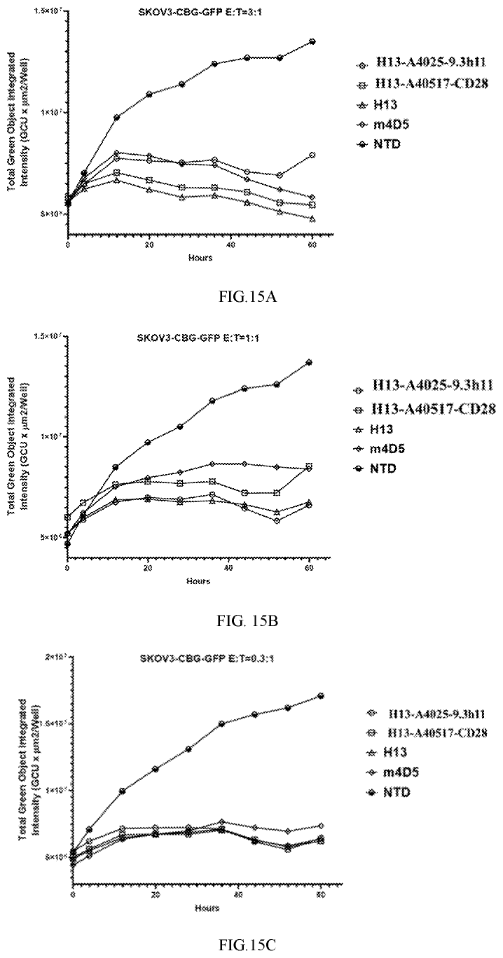

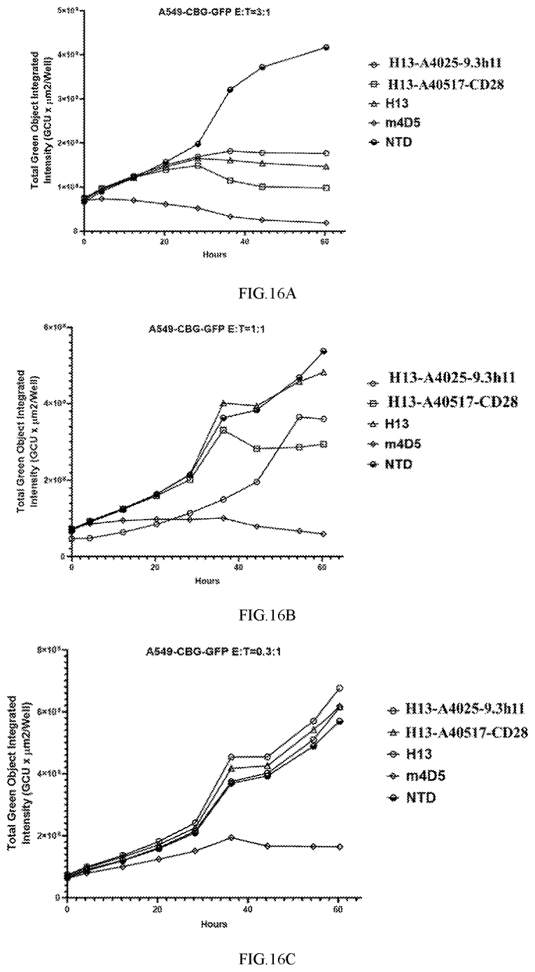

1. Twelve hours prior to co-culture for cytotoxicity experiments with CAR-T cells, seed tumor cells into a flat-bottom 96-well plate at a density of 3,000 cells/100 μl per well.
2. After 8-12 hours, allow the cells to fully adhere to the well surface. Dilute various anti-HER2 CAR-T cells to appropriate cell densities and co-incubate them with tumor cells at different effector-to-target ratios (such as E:T=3:1, 1:1, and 0.3:1).
3. Place the 96-well plate into the InCucyte S3 machine and set the scanning parameters.
4. After 3 days of scanning, analyze the total green fluorescence cumulative intensity (GCU×μm²/well) to calculate tumor cell killing efficiency. The results are shown in FIG. 14-17. Compared with m4D5 cells (anti-HER2 CART cells), H13 CAR-T cells, and 2 types of HER2 CAR/LACO CAR-T cells, such as H13-A40517-CD28 and H13-A4025-9.3h11 CAR-T cells, have minimal killing effect on A549 cells with low HER2 expression levels (see FIG. 16).

### Example 13 Preparation of Lentiviral Anti-CDH17 CAR-T Cells

The lentiviral vector was digested with XbaI and SalI enzymes and purified by gel purification method. The anti-CDH17 CAR (SEQ ID NO: 197, anti-CDH17 CAR-CD20 (CD20 mimotope was added as a switch for safety having an exemplary amino sequence of SEQ ID NO: 205) and anti-CDH17 CAR-CD20.LACO (an exemplary amino sequence of LACO shown in SEQ ID NO: 186) sequences were amplified by PCR and purified by gel purification method.

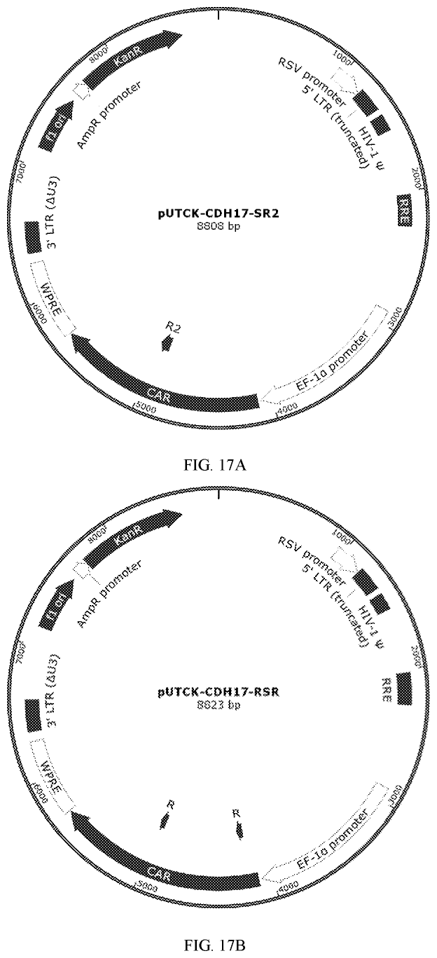

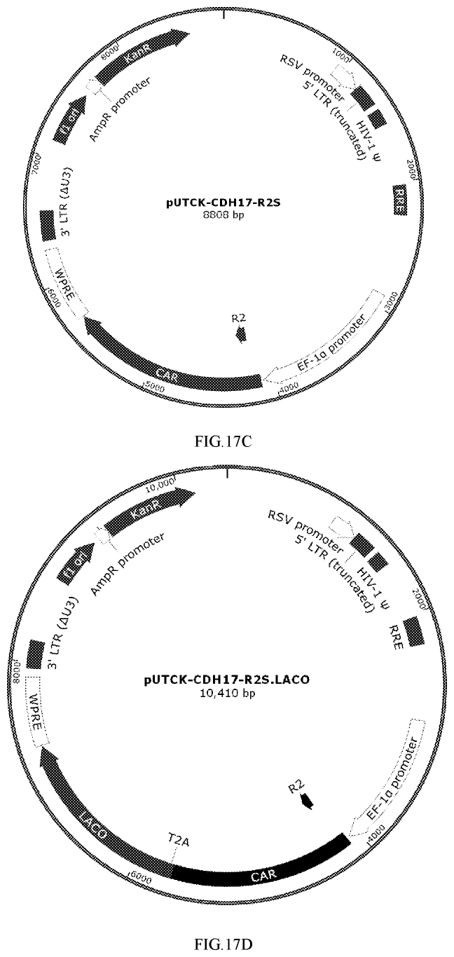

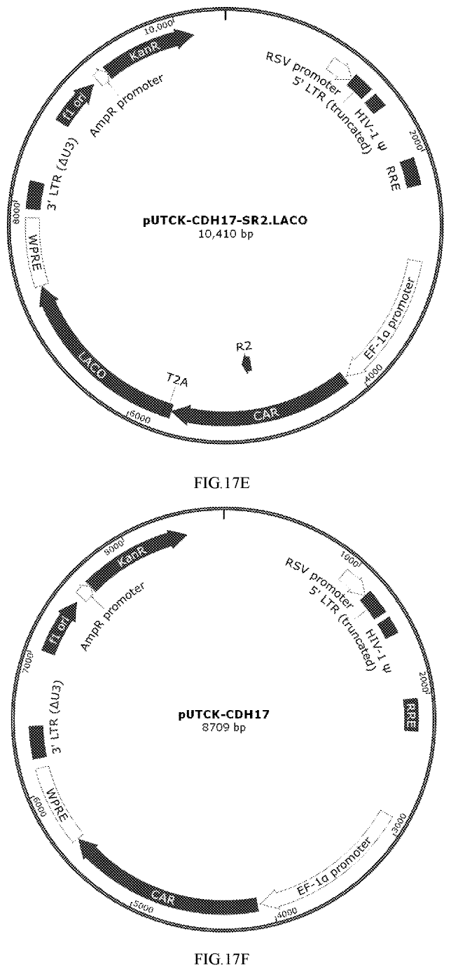

To detect whether CD20 insert location will affect the function of the CAR, we designed three different forms about anti-CDH17 CAR-CD20: anti-CDH17-SR2 CAR (two CD20 mimotope domains after CDH17 scFv having an exemplary amino sequence of SEQ ID NO: 202, FIG. 17A), anti-CDH17-R2S CAR (two CD20 mimotope domains ahead of CDH17 scFv having an exemplary amino sequence of SEQ ID NO: 203, FIG. 17C) and anti-CDH17-RSR CAR (CDH17 scFv between two CD20 mimotope domains having an exemplary amino sequence of SEQ ID NO: 204, FIG. 17B). Anti-CDH17 CAR (SEQ ID NO: 197, FIG. 17F) T cells and anti-CDH17 CAR-CD20 T cells co-expressed with LACO were also constructed: anti-CDH17-SR2 CAR.LACO (SEQ ID NO: 200, FIG. 17E), anti-CDH17-R2S CAR.LACO (SEQ ID NO: 201, FIG. 17D). Fragments and vectors are linked by homologous recombination and transformed to competent cells. The right colonies, confirmed by sanger sequencing, were selected for further experiment. FIG. 17A-17F provide all the 6 schematic representation of the vectors we used. Then the lentivirus was packed and after 30 hours the supernatant was collected and centrifuged.

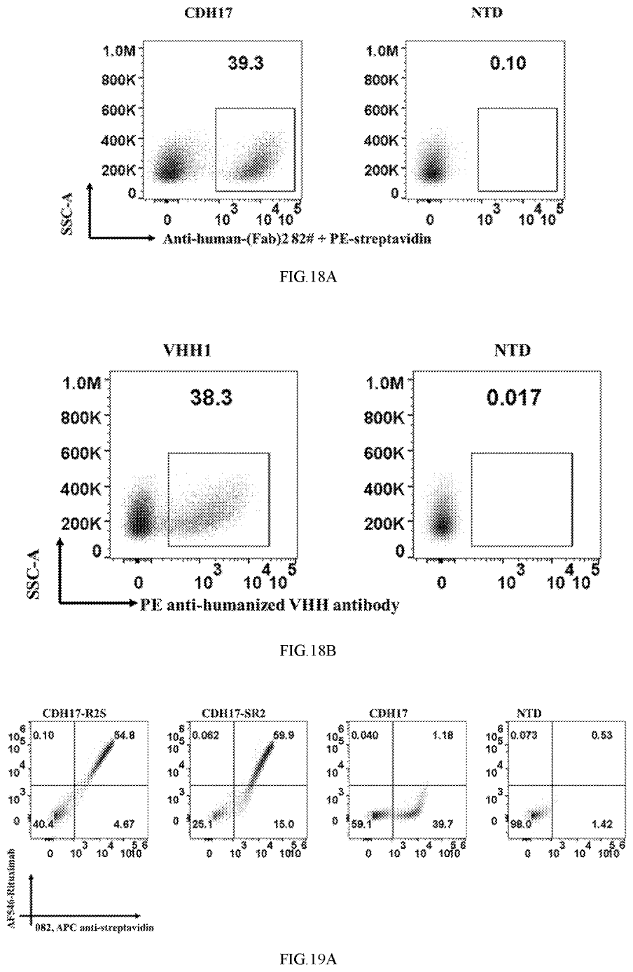

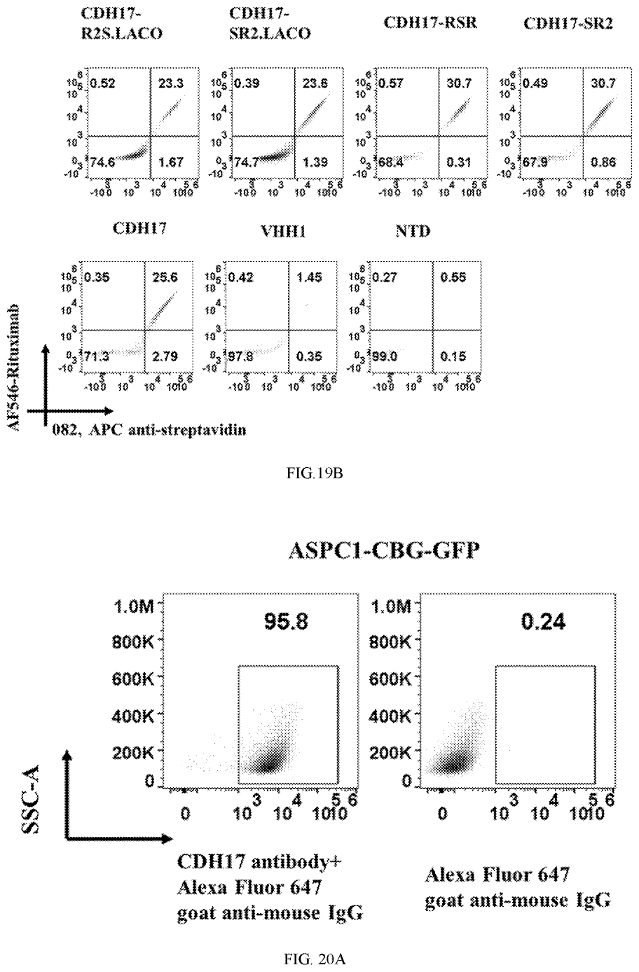

Lentivirus-based CART cells were generated using the following procedures: T cells were isolated from PBMC and activated by anti-CD3/CD28 beads (T cell:beads=1:3). At day 1, the activated T cells were transduced with CDH17 CAR, CDH17-CD20 or CDH17-CD20.LACO lentivirus at a multiplicity of infection (MOI) of 3. At day 9, the transduction efficiency of T cell was evaluated by FACS staining. NTD was control T cells without CAR lentivirus, and VHH1 (Amino sequence of VHH1 CAR is shown in SEQ ID NO: 243 and its nucleotide sequence is shown in SEQ ID NO: 244, which is published in PCT publication WO2019/210155A1) was also used as a control for T cells with CAR lentivirus in these experiments. The staining results (FIG. 18A-18B and FIG. 19A-19B) showed that the CAR molecules on anti-CDH17 CAR-T cells were effectively expressed.

Rituximab could combine with CD20, so we used rituximab to test whether the CD20 in our constructs was functional with two methods. Method 1: We stained the cells with AF546-Rituximab, washed two times with PBS, then stained with Biotin-SP-conjugated AffiniPure goat anti-human IgG-(Fab)2 082 protein (from Jackson Immuno Research, referred to as “082” in the following text), the result showed that the CDH17-R2S CAR T cells and CDH17-SR2 CAR T cells could combine with rituximab, while CDH17 CAR T cells which construct had no CD20 could not combine with rituximab (FIG. 19A). Method 2: CAR T cells were stained with both 082 and AF546-Rituximab, the result showed that all the five CDH17 CAR T cells, except VHH1, exhibited nonspecific staining with AF546-Rituximab (FIG. 19B). So, we chose the method 1 to detect the CD20 expression. The CART cells were cultured up to day 14, which were used for functional study immediately or frozen and stored using liquid nitrogen.

### Example 14 Tumor Killing of Anti-CDH17 CART Cells Transduced by Lentivirus

The cytolytic activities of the provided CDH17 CARTs cells from Example 13 were measured in the tumor killing assay. T cells expressing CDH17, CDH17-CD20 or CDH17-CD20.LACO were co-cultured with tumor cells, at E/T ratio=3:1, 1:1 and 0.3:1. NTD was control T cells without CAR lentivirus, and VHH1 was also as control T cells with CAR lentivirus in these experiments.

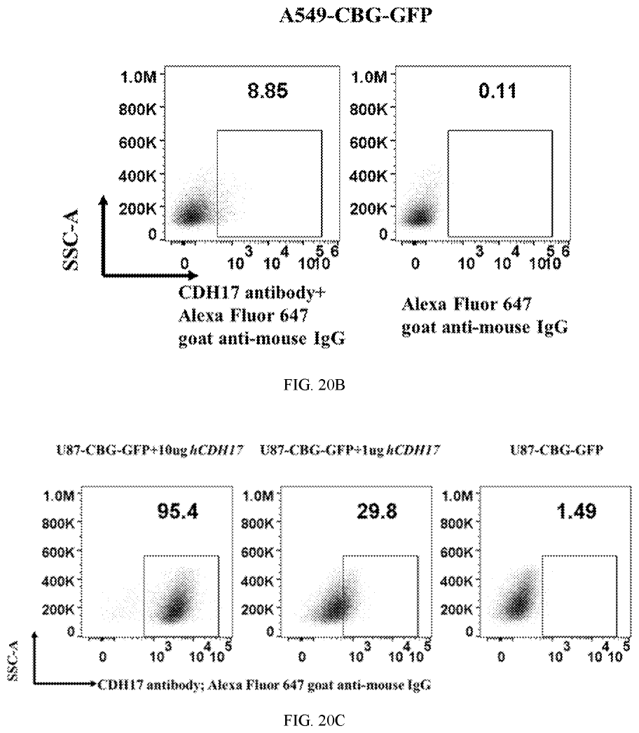

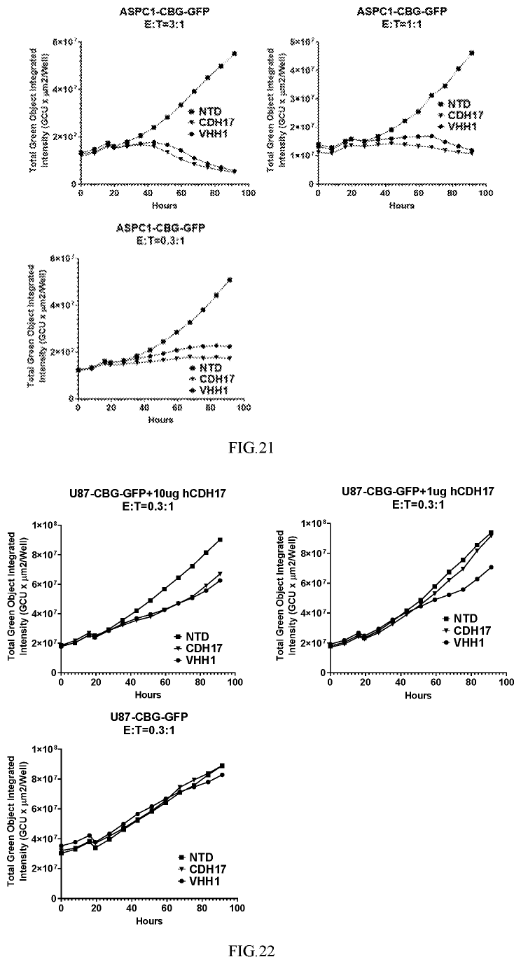

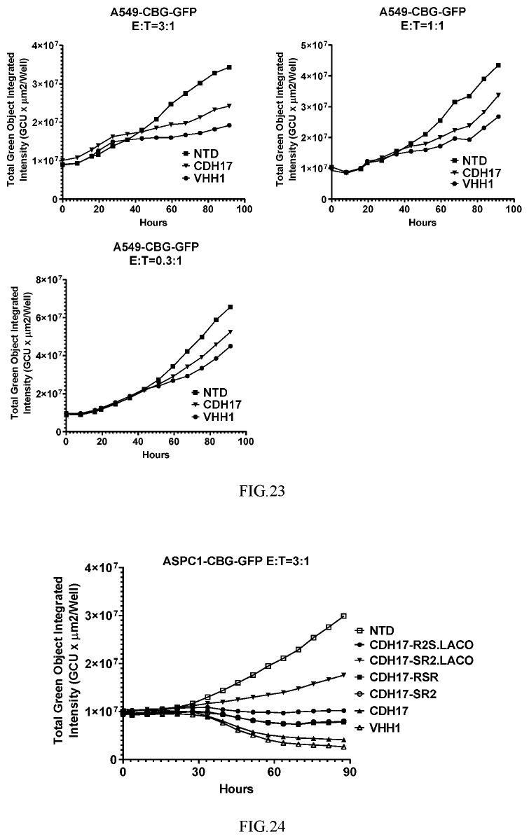

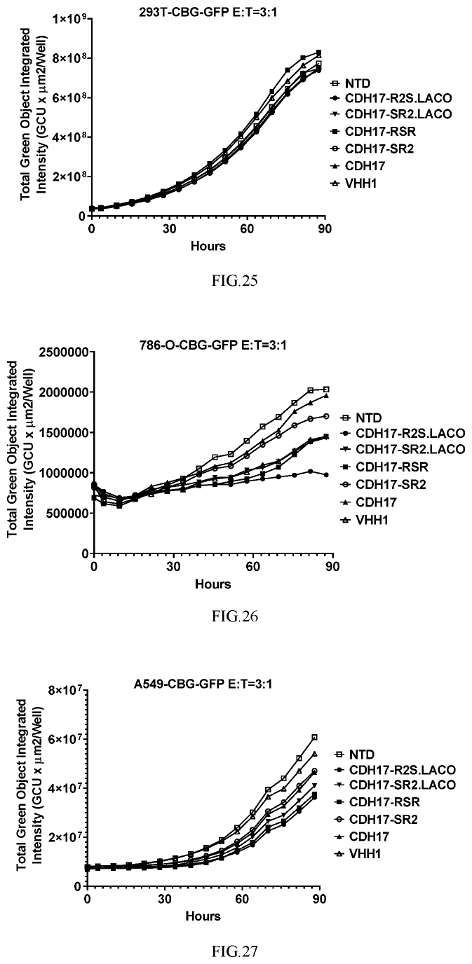

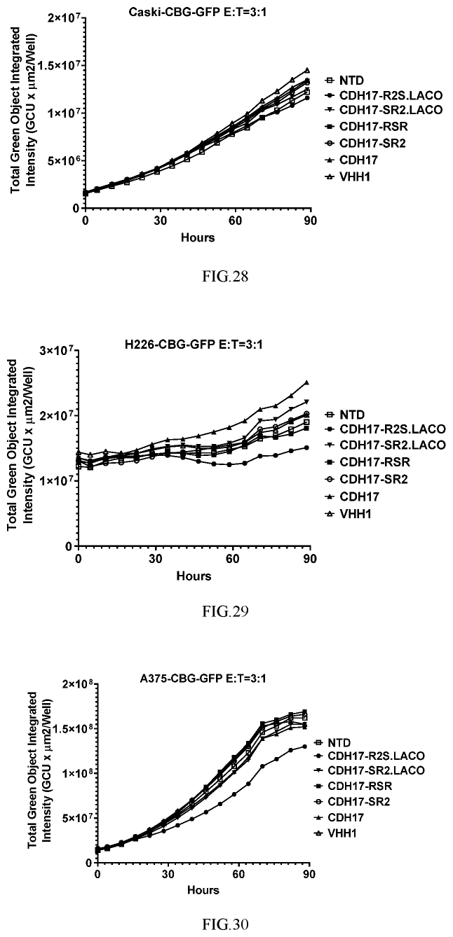

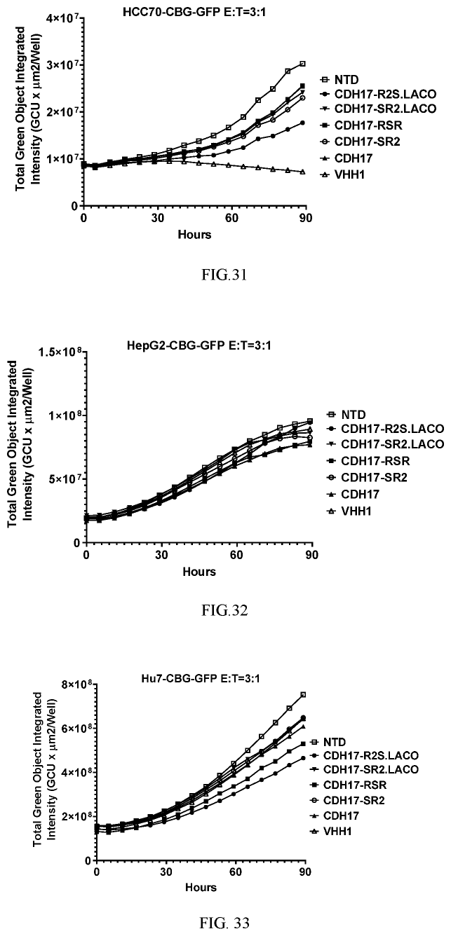

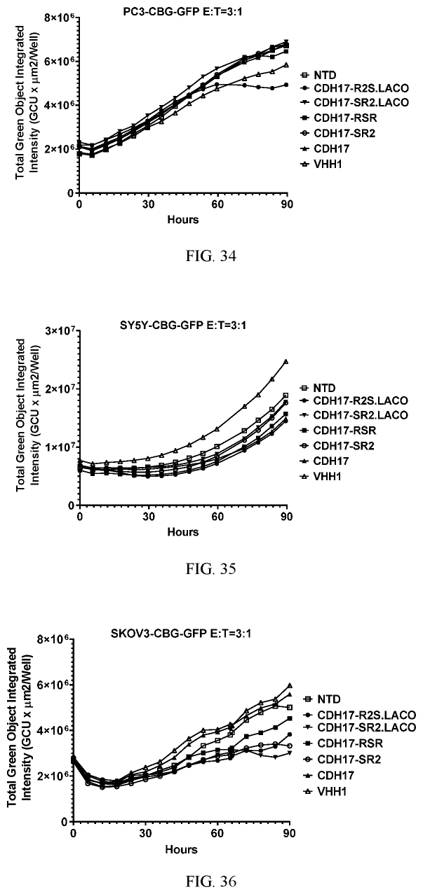

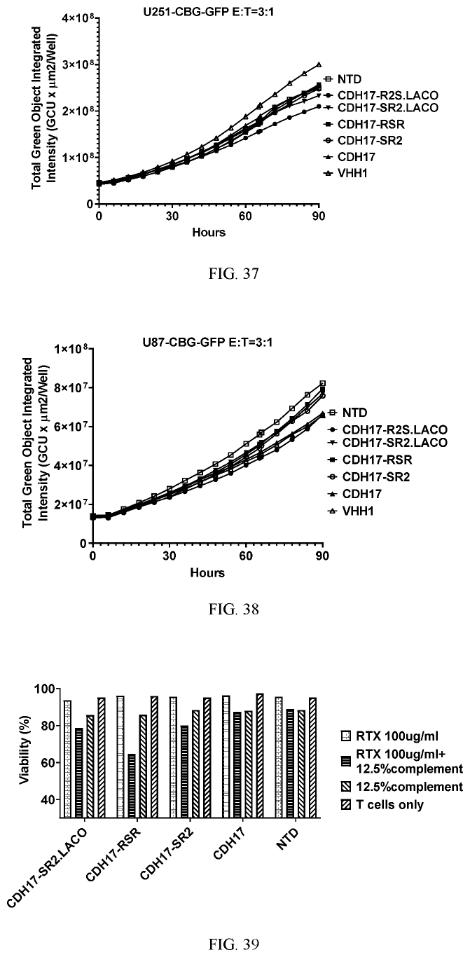

To know the expression of CDH17 antigen in tumor cells, we stained ASPC1-CBG-GFP (FIG. 20A), A549-CBG-GFP (FIG. 20B) and U87-CBG-GFP (U87-CBG-GFP cells only or U87-CBG-GFP cells electroporated with hCDH17 mRNA) (FIG. 20C) tumor cells with CDH17 antibody. CDH17 CAR T cells could effectively kill ASPC1 (FIG. 21) and U87+10 μg hCDH17 tumor cells (FIG. 22) with high expression of CDH17 antigen. For A549 and U87+1 μg hCDH17 cells, which weakly expressing CDH17, the killing ability of CDH17 CAR T cells was similar to that of control VHH1 cells (FIG. 22 and FIG. 23) and other killing curves were showed in FIG. 24-38 with tumor cells of ASPC1 (FIG. 24), 293T (FIG. 25), 786-O (FIG. 26), A549 (FIG. 27), Caski (FIG. 28), H226 (FIG. 29), A375 (FIG. 30), HCC70 (FIG. 31), HepG2 (FIG. 32), Hu-7 (FIG. 33), PC3 (FIG. 34), SY5Y (FIG. 35), SKOV3 (FIG. 36), U251 (FIG. 37) and U87 (FIG. 38).

### Example 15 the Effect of RTX and Complement on CAR T-Cells Viability

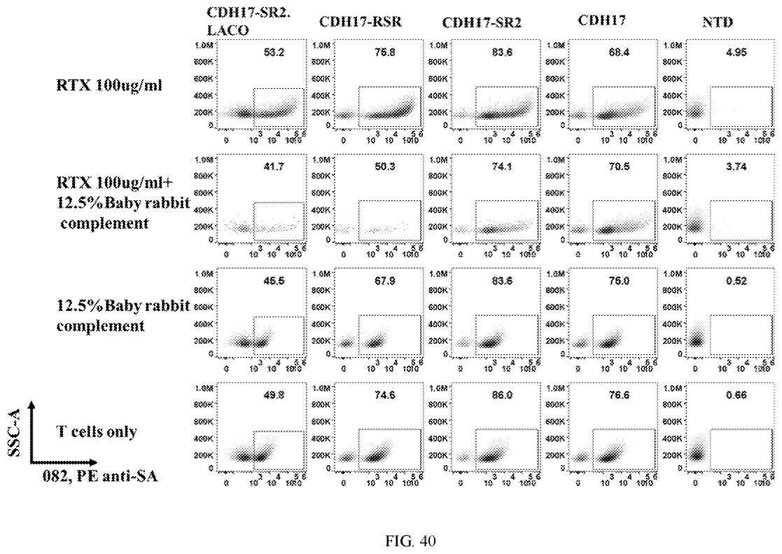

To assess the effect of RTX (Rituximab Injection, Henlius) and complement (baby rabbit complement, Pel-Freez, 31061-1) on CAR T-cells viability, 2×10⁵ CAR T-cells were incubated alone or in the presence of 100 μg/mL RTX and complement (diluted 8 times from a 10 ml stock solution of complement) in a final volume of 400 μL. After 90 min, cells were washed with PBS and detected the live rate by NC250. The viability of CAR T cells constructed with CD20 was lower in the group of RTX+ complement than the group of complement. For CDH17 and NTD cells, there were no difference in these two groups (FIG. 39). The remaining cells were analyzed by flow cytometry to determine the binding rate of live cells to Biotin-SP-conjugated AffiniPure goat anti-human IgG-(Fab)2 309-065-082 (hereinafter referred to as 082) protein. Under the treatment of RTX+ complement, RSR CAR T cells had lower positive live cells than SR2 cells (FIG. 40). The position of CD20 (see FIG. 17) may affect the combination of RTX with CD20 CAR T cells.

### Example 16 Preparation of Lentiviral Anti-CD70 CAR-T Cells

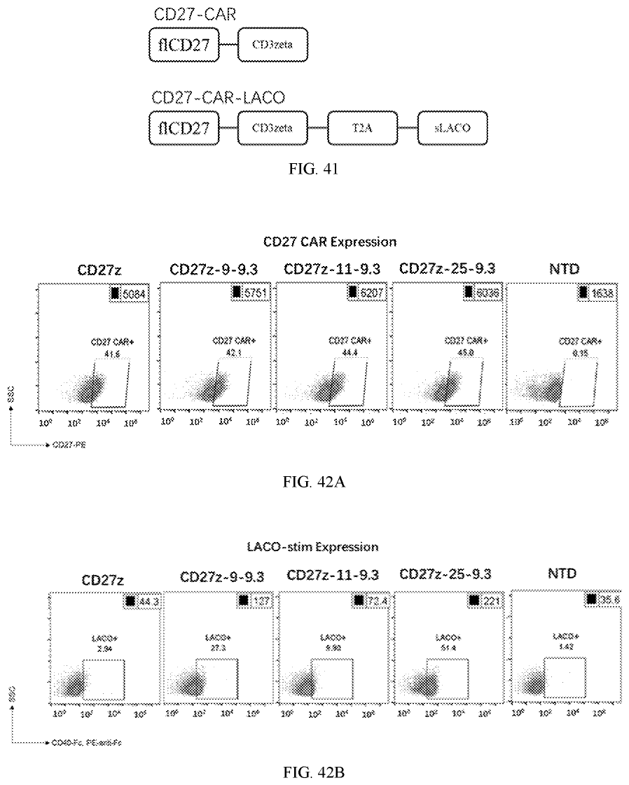

CD27 is a natural ligand for CD70. In this study, CARs targeting CD70, including CD27-CAR (referred as CD27z in the present application, SEQ ID NO: 195) and CD27-CAR-LACO were constructed. As shown in FIG. 41, CD27-CAR was composed of CD27-full length (FL) and the ζ domain of CD3. CD27-CAR-LACO was composed of CD27-FL, the ζ domain of CD3, T2A and LACO. Different LACOs were co-expressed with the CD27-CAR, the information of CD27-CAR and CD27-CAR-LACOs is shown in the Table 9.

TABLE 9
CD27-CAR and CD27-CAR-LACOs

| CAR/CAR-LACO | LACO | Amino Acid Sequence |
|---|---|---|
| CD27z | — | SEQ ID NO: 195 |
| CD27z-9-9.3 | A409-9.3h11 | SEQ ID NO: 261 |
| CD27z-11-9.3 | A4011-9.3h11 | SEQ ID NO: 262 |
| CD27z-25-9.3 | A4025-9.3h11 | SEQ ID NO: 196 |

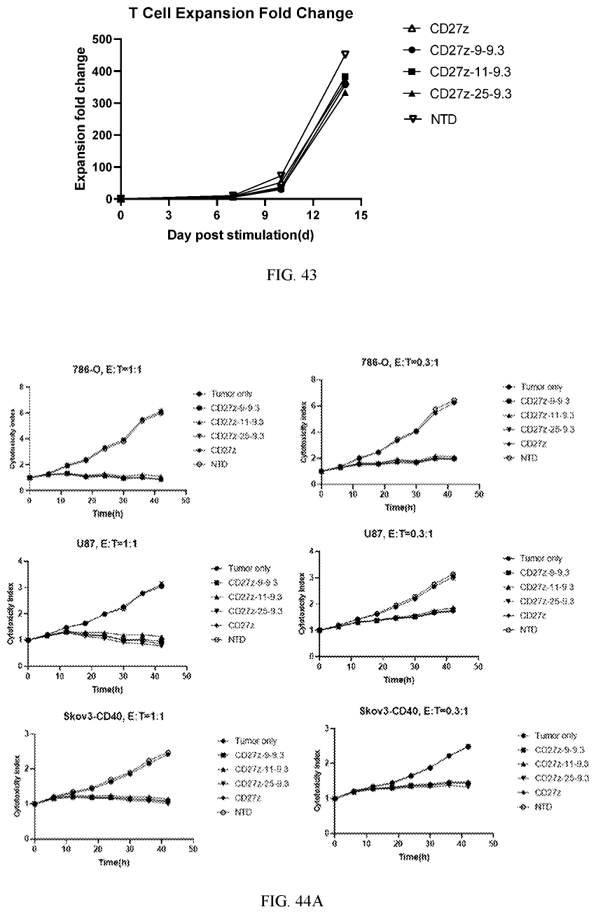

Lentivirus-based CART cells were generated using the following procedures: T cells were isolated from PBMC and activated by anti-CD3/CD28 beads (T cell:beads=1:3). At day 1, T cells were transduced with lentiviral vectors carrying CD27z, CD27z-9.9.3, CD27z-11.9.3, CD27z-25.9.3 at a multiplicity of infection (MOI) of 3. At day 13, the CAR expression was detected using anti-CD27 antibody, and the expression of LACO was detected using CD40-Fc (FIG. 42A-42B and Table 10). Generally, the transduction efficiency was between 30% to 50%. The CAR-T cells were cultured up to day 14, there was no significant expansion difference in CD27z, CD27z-9.9.3, CD27z-11.9.3, CD27z-25.9.3 and NTD T cell as shown in FIG. 43.

TABLE 10
CAR %, LACO % and Medium Fluorescence
Intensity (MFI) of Expression

| CAR/CAR-LACO | CAR % | CAR/MFI | LACO % | LACO/MFI |
|---|---|---|---|---|
| CD27z | 28.1 | 5076 | 2.9 | 44.3 |
| CD27z-9-9.3 | 38.1 | 7972 | 27.3 | 127 |
| CD27z-11-9.3 | 44.9 | 10714 | 9.9 | 72.4 |
| CD27z-25-9.3 | 43.3 | 10043 | 51.4 | 221 |

### Example 17 Tumor Killing of Anti-CD70 CAR-T Cells Transduced by Lentivirus

The tumor killing effects of the CAR T cells were measured. CAR T cells including mock T cells (NTD), CD27z, CD27z-9.9.3, CD27z-11.9.3, CD27z-25.9.3 were co-cultured with different cancer cells, including 786-O, U87, A549, HepG2 and Skov3-CD40(transduced with CD40) at different Effect (E): Target (T) ratio (3:1, 1:1, 0.3:1). Killing curves were analyzed in Incucyte for 48 h. As shown in FIG. 44A, CD27z, CD27z-9.9.3, CD27z-11.9.3, CD27z-25.9.3 CART cells showed similar levels of cytotoxicity against CD70 high expression tumor cells 786-O, U87 and Skov3-CD40 at E/T ratio as 1:1 and 0.3:1. As shown in FIG. 44B, CD27z, CD27z-9.9.3, CD27z-11.9.3, CD27z-25.9.3 CART cells had low killing effect toward the A549 and HepG2 tumor cells with low CD70 expression at E/T ratio as 3:1 and 1:1.

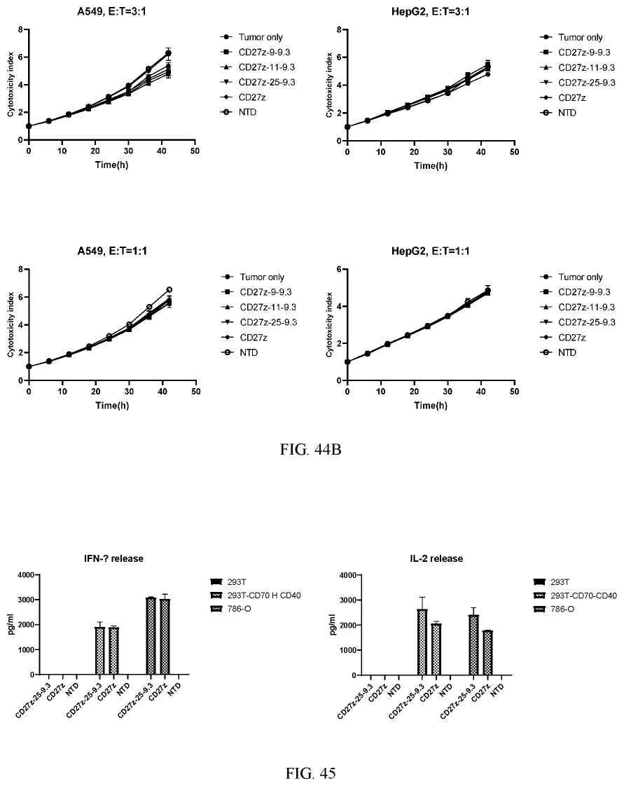

In further functional evaluation, CD27z, CD27z-25.9.3 CAR-T cells were incubated with 786-0, 293T and 293T-CD70/CD40 (electroporated with CD70 and CD40 mRNA) with E:T=1:1 for 24 h, and the production of IFN-γ levels and IL-2 levels in the supernatant were detected by ELISA. As shown in FIG. 45, CD27z and CD27z-25-9.3 CAR T cells showed similar IFN-γ release against CD70 786-O and 293T-CD70/CD40 tumor cells. CD27z-25-9.3 CAR T cells enhanced IL-2 release compared to CD27z CAR T cells against CD70 786-O and 293T-CD70/CD40 tumor cells. It suggested LACO (A4025-9.3 h11) improved IL-2 cytokine release compared to no LACO CAR T cells.

### Example 18 CO-Expression of LACO Activated CD40+ Cell Lines

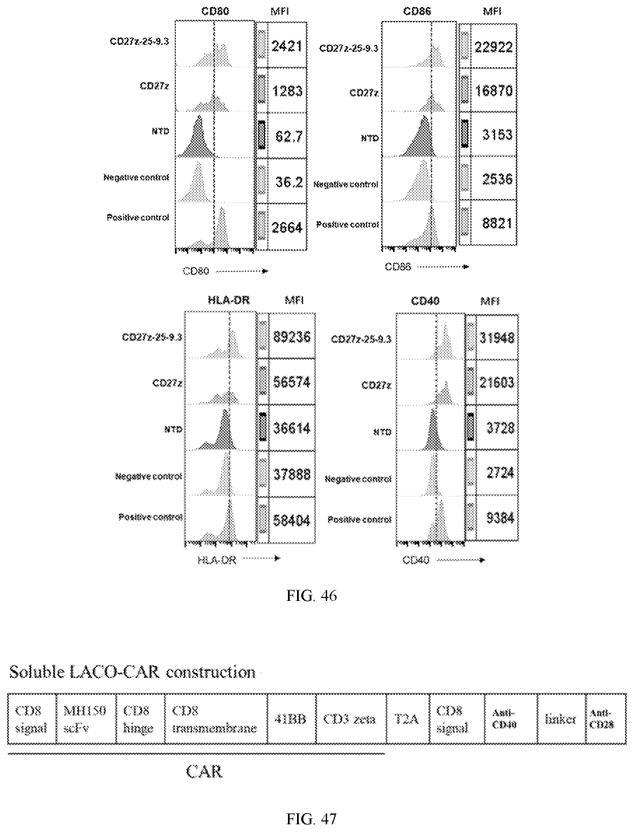

A co-culture of CD27z, CD27z-25.9.3 CART cells, mock T cells (NTD), negative control and positive control with CD40+ B-cell tumor cell DOHH2 was performed. Cultures with CD27z-25.9.3 CART led to the upregulated expression costimulatory molecules (CD80, CD86, CD83) and CD40 on the surface of DOHH2 cell line, compared to CD27z CART and NTD T cells as shown in FIG. 46. In this co-culture assay, negative control was no CAR T cells added, positive control was mixtures of IFNγ (1000U/ml), R848(2.5ug/ml), PolyIC (20ug/ml) and LPS (100 ng/ml).

### Example 19 Preparation and Characterization of Anti-CD70 CAR-Ts by Lentiviral Transduction

The antigen-binding domain of an anti-CD17 CAR can comprises an antibody or antigen-fragment thereof. The vector was constructed to produce the LACO and anti CD70-CAR co-expressing lentiviral. First, the LACO sequence (for example, the amino acid sequence of LACO-A4D11C2828 as set forth in SEQ ID NO: 263, the amino acid sequence of LACO-Al 19C2828 as set forth in SEQ ID NO: 264) and the anti-CD70-CAR sequence (as set forth in SEQ ID NO: 111) were amplified separately by PCR. For example, the LACO sequence consists of the sequence of anti-CD40 scFv, CD28 extracellular domain, CD28 transmembrane domain and CD28 intracellular domain. For example, the anti-CD70 CAR sequence consists of the sequence of anti-CD70 scFv and CAR region (including hinge region to CD3-zeta region). The LACO sequence and the anti-CD70 CAR sequence were linked by an F2A peptide and were subcloned into the pUTCK lentiviral packaging vector.

First, 293T cells were plated in a 150 mm culture dish at a density of 1.3×10E7 cells/dish, and cultured at 37° C. for 16 hours in a 5% CO2 incubator. The next day, the lentiviral plasmid pUTCK-CAR and auxiliary packaging plasmids (pRSV.REV, pMD2g and pMDLg-pRRe) were co-transfected into 293T cells by PEI, and the cells culture supernatant were harvested at 24 hours and 48 hours after transfection. The cell culture supernatant was collected by ultra-high-speed centrifugation to collect the virus particle pellet, and the virus pellet was resuspended in fresh medium to obtain lentivirus.

Titer determination of lentivirus. Dilute the lentivirus 3-fold with fresh medium, take 50 μl of the lentivirus solution and incubate with the SupT1 cell suspension (20,000 cells/100 l) for 48-72 hours. The titer of lentivirus was detected by flow staining.

The lentiviral transduced LACO-anti-CD70 CAR-Ts were prepared by the following steps: naive CD3+ T cells were first activated with Dynabeads™ Human T Activator CD3/CD28 (Thermo, #11161D) for T cell activation and expansion. LACO-anti-CD70 CAR lentivirus was added according to MOI=1, and after continuous infection for 3-5 days, Dynabeads and T cells were separated with magnetic separation device. The T cell density was adjusted to 5×10E5 cells/ml with the R10 medium containing IL-2 (100U/ml), and was cultured continually with the R10 medium containing IL-2 for 5-7 days, fresh R10 medium containing IL-2 was added every other day.

The binding of LACO-anti-CD70 CAR-T cells produced by lentiviral transduction and CD70-Fc recombinant protein or CD40-Fc recombinant protein was measured by FACS staining. As shown in FIG. 49, T cells derived from the healthy donor ND022 (purchased from Milestone Biotechnologies, ID: DS211333) expressing anti-CD70 CAR (70.202) can bind to CD70-Fc recombinant protein, and expressing LACO-anti-CD70 CAR (A40C2828-70.202) containing CD40 scFv can bind to CD40-Fc recombinant protein. Untransduced (UTD) are control T cells without CAR molecules.

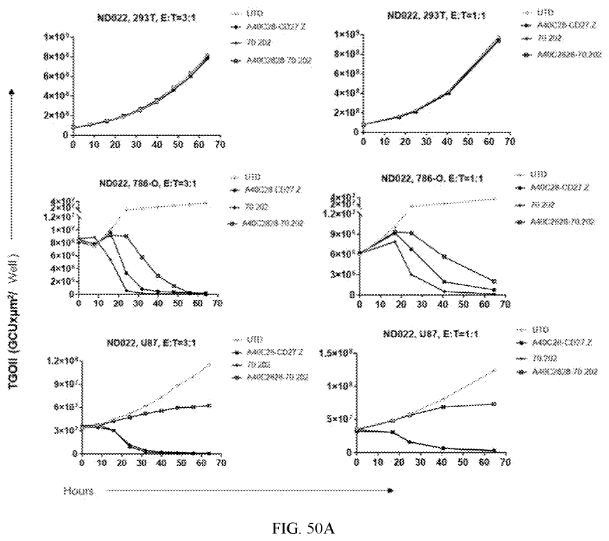

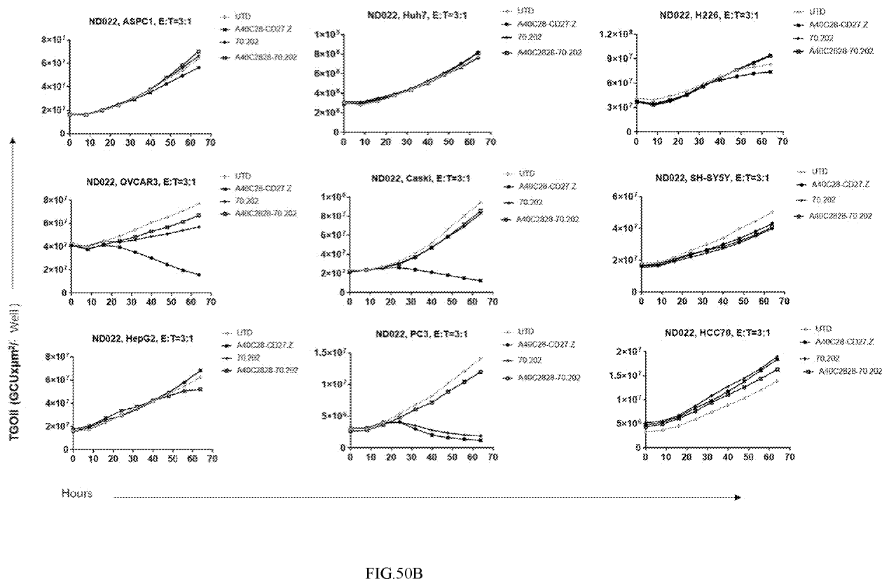

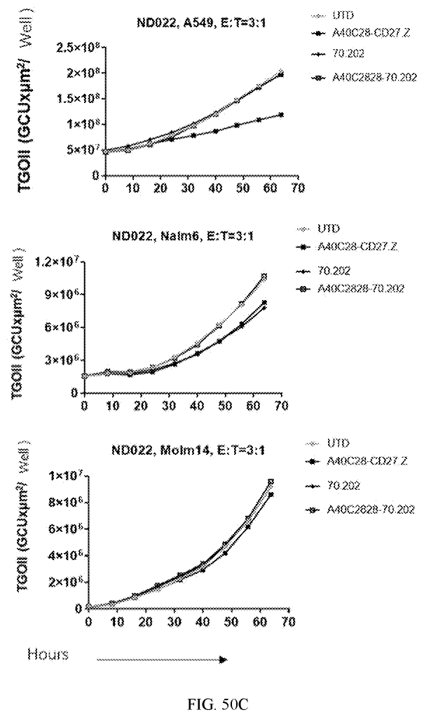

FIG. 50A-50C show the killing curves of T cells derived from the healthy donor ND022 expressing LACO-anti-CD70 CAR by lentiviral transduction to different tumor cells at the E/T ratio of 1:3 or 1:1. As shown in the figure, CAR-T cells expressing LACO-anti-CD70 CAR effectively prevented the growth of tumor cells (e.g., 786-O, U87, OVCAR3 and PRMI8226) expressing CD70 at medium or high level endogenously, and even eliminated these tumor cells.

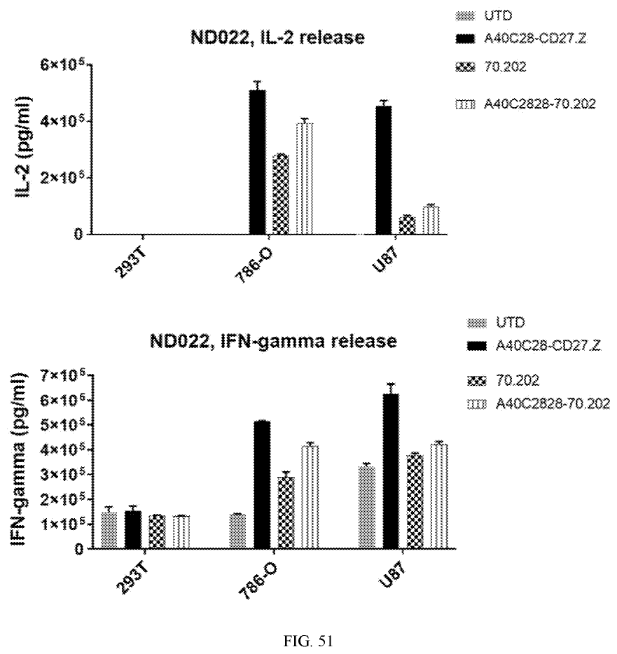

FIG. 51 shows the level of released cytokine IL-2 and IFN-gamma in cell supernatant after co-incubation of T cells derived from the healthy donor ND022 expressing LACO-anti-CD70 CAR by lentiviral transduction and tumor cells (293T, 786-O, U87) with different expression levels of endogenous CD70 of 24h. As shown in the figure, after co-incubation with tumor cells that did not express CD70 endogenously, LACO-anti-CD70 CAR-T cells did not release IL-2 and released low levels of IFN-gamma. After co-incubation with tumor cells expressing CD70 at medium or high level (786-O and U87), except for A40C28-CD27.Z, A40C2828-70.202 CART released higher levels of IL-2 and IFN-gamma.

### Example 20 Application of Anti-CD70 CAR-Ts by Lentiviral Transduction in 786-O Tumor Model

Severe immunodeficiency M-NCG mice (purchased from Shanghai Model Organisms Center, Inc) were subcutaneously inoculated with 10×10⁶ renal cell carcinoma cells 786-O. On the 13th day after tumor inoculation, 0.6×10⁶ 70.202 CAR-T cells, A4D 11C2828-70.202 CAR-T cells, and Al 19C2828-70.202 CAR-T cells were intravenously injected, and the UTD group was the blank control group that injected with T cells. Cell killing capacity, cytokine secretion, and tumor volume were measured.

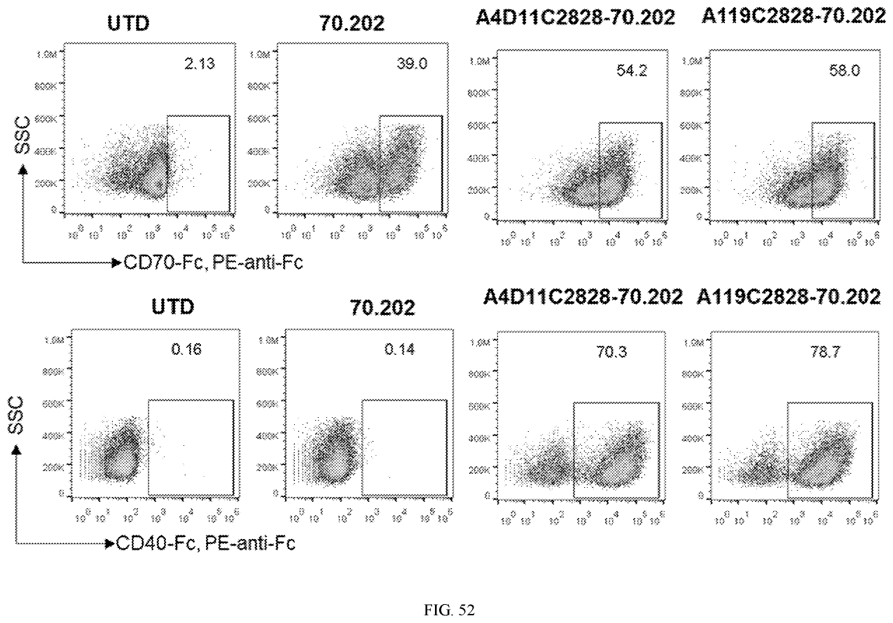

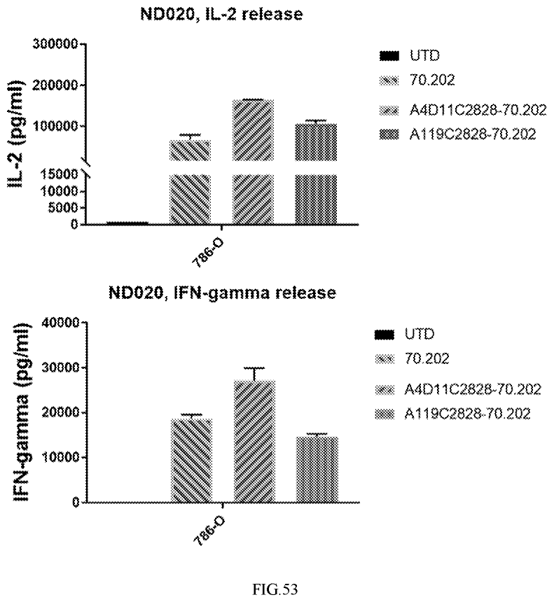

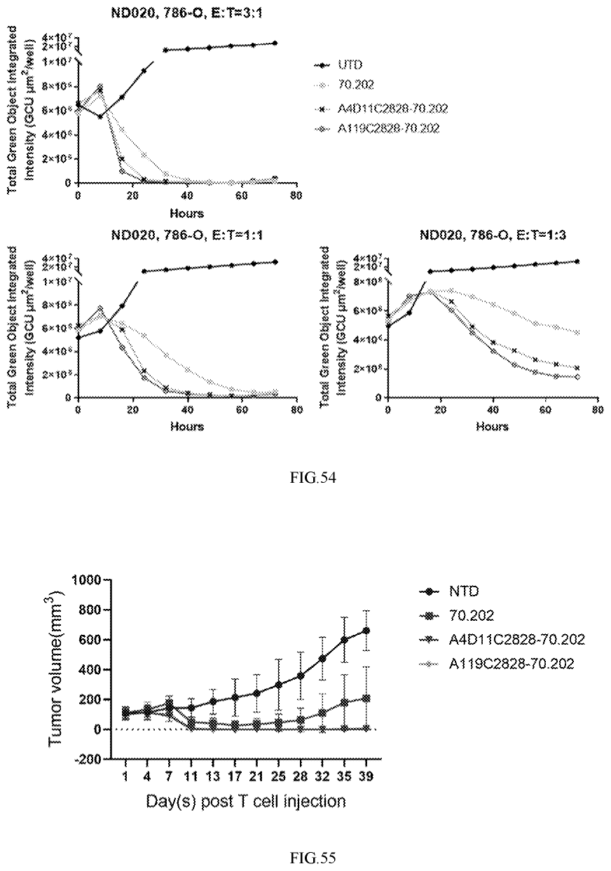

FIG. 52 shows that both anti-CD70 CAR and LACO were efficiently expressed. FIG. 54 shows that compared with the UTD control T cell group, 70.202 CAR-T cells, A4D11C2828-70.202 CAR-T cells, and A119C2828-70.202 CAR-T cells can effectively control the growth of tumors, and the cell killing capacity of cells co-expressing CAR and LACO were further improved. FIG. 53 shows that compared with 70.202 CAR-T cells, the secretion of cytokines IL-2, IFN-gamma of A4D11C2828-70.202 CAR-T cells and A119C2828-70.202 CAR-T cells have been enhanced. FIG. 55 shows that 70.202 CAR-T cells, A4D11C2828-70.202 CAR-T cells and A119C2828-70.202 CAR-T cells can effectively control tumor volume, and the expression of LACO enhances tumor killing ability.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. An antigen-binding protein that specifically binds CD40, comprising a heavy chain variable region (VH) and a light chain variable region (VL), wherein the VH comprising a heavy chain CDR1 (HCDR1), a heavy chain CDR2 (HCDR2) and a heavy chain CDR3 (HCDR3); and the VL comprising a light chain CDR1 (LCDR1), a light chain CDR2 (LCDR2) and a light chain CDR3 (LCDR3), wherein
a) the HCDR1, HCDR2 and HCDR3 comprise the amino acid sequence as set forth in SEQ ID NOs: 2, 3 and 4, respectively; and the LCDR1, LCDR2 and LCDR3 comprise the amino acid sequence as set forth in SEQ ID NOs: 6, 7, and 8, respectively;
b) the HCDR1, HCDR2 and HCDR3 comprise the amino acid sequence as set forth in SEQ ID NOs: 11, 12 and 13, respectively; and the LCDR1, LCDR2 and LCDR3 comprise the amino acid sequence as set forth in SEQ ID NOs: 15, 16, and 17, respectively;
c) the HCDR1, HCDR2 and HCDR3 comprise the amino acid sequence as set forth in SEQ ID NOs: 2, 3 and 20, respectively; and the LCDR1, LCDR2 and LCDR3 comprise the amino acid sequence as set forth in SEQ ID NOs: 22, 23, and 24, respectively;
d) the HCDR1, HCDR2 and HCDR3 comprise the amino acid sequence as set forth in SEQ ID NOs: 27, 28 and 29, respectively; and the LCDR1, LCDR2 and LCDR3 comprise the amino acid sequence as set forth in SEQ ID NOs: 31, 32, and 33, respectively;
e) the HCDR1, HCDR2 and HCDR3 comprise the amino acid sequence as set forth in SEQ ID NOs: 36, 37 and 38, respectively; and the LCDR1, LCDR2 and LCDR3 comprise the amino acid sequence as set forth in SEQ ID NOs: 40, 41, and 42, respectively;
f) the HCDR1, HCDR2 and HCDR3 comprise the amino acid sequence as set forth in SEQ ID NOs: 45, 46 and 47, respectively; and the LCDR1, LCDR2 and LCDR3 comprise the amino acid sequence as set forth in SEQ ID NOs: 49, 50, and 51, respectively;
g) the HCDR1, HCDR2 and HCDR3 comprise the amino acid sequence as set forth in SEQ ID NOs: 54, 55 and 56, respectively; and the LCDR1, LCDR2 and LCDR3 comprise the amino acid sequence as set forth in SEQ ID NOs: 58, 41, and 59, respectively;
h) the HCDR1, HCDR2 and HCDR3 comprise the amino acid sequence as set forth in SEQ ID NOs: 62, 63 and 64, respectively; and the LCDR1, LCDR2 and LCDR3 comprise the amino acid sequence as set forth in SEQ ID NOs: 66, 67, and 68, respectively; or
i) the HCDR1, HCDR2 and HCDR3 comprise the amino acid sequence as set forth in SEQ ID NOs: 45, 46 and 47, respectively; and the LCDR1, LCDR2 and LCDR3 comprise the amino acid sequence as set forth in SEQ ID NOs: 71, 72, and 51, respectively.

2. The antigen-binding protein of claim 1, wherein
a) the VH comprising at least 85%, at least 90%, at least 95%, at least 98%, or 100% sequence identity to an amino acid sequence as set forth in any one of SEQ ID NOs: 1, 10, 19, 26, 35, 44, 53 and 61; and/or
b) the VL comprising at least 85%, at least 90%, at least 95%, at least 98%, or 100% sequence identity to an amino acid sequence as set forth in any one of SEQ ID NOs: 5, 14, 21, 30, 39, 48, 57, 65 and 70.

3. The antigen-binding protein of claim 1, wherein the antigen-binding protein is an antibody or an antigen-binding fragment.

4. The antigen-binding protein of claim 1, wherein the antigen-binding protein comprises a Fab, a Fab′, a F(ab′)2, a Fv, a scFv, a (scFv)2.

5. The antigen-binding protein of claim 1, wherein the antigen-binding protein comprises a scFv, and the scFv comprises an amino acid sequence as set forth in any one of SEQ ID NOs: 9, 18, 25, 34, 43, 52, 60, 69, and 73.

6. A fusion protein comprising a first domain and a second domain, wherein (i) the first domain comprises antigen-binding protein of claim 1; and (ii) the second domain activates an immune effector cell and comprises (a) a co-stimulatory receptor of the immune effector cell, or a functional fragment thereof, (b) a co-stimulatory ligand of the immune effector cell, or a receptor-binding fragment thereof, or (c) an antibody or antigen-binding fragment that binds a co-stimulatory receptor of the immune effector cell, or an antigen-binding fragment thereof.

7. The fusion protein of claim 6, wherein the co-stimulatory receptor comprises CD28, 4-1BB, ICOS, CD27, OX40, DAP10, 2B4, CD30, CD2, LIGHT, GITR, TLR, DR3, and/or CD43; the co-stimulatory ligand comprises CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R, and/or CD44.

8. The fusion protein of claim 6, wherein the second domain comprises an antibody or an antigen-binding fragment that specifically binds CD28 and comprises a heavy chain variable region (VH) and/or a light chain variable region (VL), wherein the VH comprises a heavy chain CDR1 (HCDR1) comprising the amino acid sequence as set forth in SEQ ID NO: 177, a heavy chain CDR2 (HCDR2) comprising the amino acid sequence as set forth in SEQ ID NO: 178, a heavy chain CDR3 (HCDR3) comprising the amino acid sequence as set forth in SEQ ID NO: 179; wherein the VL comprises a light chain CDR1 (LCDR1) comprising the amino acid sequence as set forth in SEQ ID NO: 180, a light chain CDR2 (LCDR2) comprising the amino acid sequence as set forth in SEQ ID NO: 181, a light chain CDR3 (LCDR3) comprising the amino acid sequence as set forth in SEQ ID NO: 182.

9. The fusion protein of claim 8, wherein the VH of the second domain comprising the amino acid sequence as set forth in any of SEQ ID NO: 183, and the VL of the second domain comprising the amino acid sequence as set forth in any of SEQ ID NO: 184.

10. The fusion protein of claim 8, wherein the first domain and/or the second domain comprises a scFv.

11. The fusion protein of claim 10, wherein the first domain comprises the amino acid sequence as set forth in SEQ ID NO: 9, 18, 25, 34, 43, 52, 60, 69, 73, 82 or 91, and the second domain comprises the amino acid sequence as set forth in SEQ ID NO: 96 or SEQ ID NO: 185.

12. The fusion protein of claim 6, wherein the N-terminus of the first domain is linked to the C-terminus of the second domain or the N-terminus of the second domain is linked to the C-terminus of the first domain.

13. The fusion protein of claim 6, wherein the first domain and the second domain are linked via a linker.

14. The fusion protein of claim 6, wherein the fusion protein comprises an amino acid sequence that is at least 85%, 90%, 95%, 98%, or 99% identical to the amino acid sequences as set forth in any one of SEQ ID NO: 155-165 or SEQ ID NO: 186.

15. A cell that comprises the fusion protein of claim 6.

16. The cell of claim 15, wherein the cell is genetically engineered and recombinantly expresses the fusion protein.

17. The cell of claim 15, wherein the cell comprises an immune effector cell.

18. The cell of claim 17, wherein the immune effector cell comprises a T cell, an NK cell, an NKT cell, a macrophage, a neutrophil, and a granulocyte.

19. The cell of claim 15, wherein the cell further comprises a chimeric antigen receptor (CAR), a T cell receptor (TCR) or a Bi-specific T-cell engager (BiTE), and/or a polynucleotide that encodes a CAR, a TCR, or a BiTE, wherein the CAR, TCR or BiTE binds a tumor antigen or a viral antigen.

20. A method for preventing and/or treating a disease or a disorder, comprising administering the cell of claim 15.
